(19) United States
(12) Patent Application Publication
(10) Pub. No.: US 2026/0022384 A1
(43) Pub. Date: Jan. 22, 2026

(54) ASPARAGINE SYNTHETASE INHIBITORS AND USES THEREOF

(71) Applicant: OSPEDALE SAN RAFFAELE S.R.L., Milano (IT)

(72) Inventors: Alessandra BOLETTA, Milano (MI) (IT); Christine PODRINI, Milano (MI) (IT); Isaline Severine Marie-Josephe ROWE, Milano (MI) (IT)

(73) Assignee: OSPEDALE SAN RAFFAELE S.R.L., Milano (IT)

(21) Appl. No.: 19/289,969

(22) Filed: Aug. 4, 2025

Related U.S. Application Data
(63) Continuation-in-part of application No. 17/272,864.

(30) Foreign Application Priority Data
Sep. 4, 2018 (EP) 18192486.1

(51) Int. Cl. C12N 15/113; A61K 31/40; A61P 13/12

(52) U.S. Cl. CPC C12N 15/1137; A61K 31/40; A61P 13/12; C12N 2310/11; C12N 2310/14; C12N 2310/3231; C12N 2310/341

(57) ABSTRACT

The present invention relates to an inhibitor of asparagine synthase for use for the treatment of a disorder characterized by renal and/or liver cyst formation and relative pharmaceutical composition.

### CROSS-REFERENCE TO RELATED APPLICATIONS

This application is a Continuation-in-part application of U.S. patent application Ser. No. 17/272,864, filed May 17, 2021, which is a 371 of PCT/EP2019/073628, filed Sep. 4, 2019, which claims the benefit of European Patent Application No. 18192486.1, filed Sep. 4, 2018, the entire content of each of which is incorporated by reference herein.

### REFERENCE TO A SEQUENCE LISTING SUBMITTED ELECTRONICALLY VIA EFS-WEB

The content of the electronically submitted sequence listing, file name: 128-1240)-CIP SeqList.txt; size: 65,577 bytes; and date of creation: Aug. 4, 2025, which is concurrently submitted with this application, is incorporated herein by reference in its entirety.

### FIELD OF THE INVENTION

The present invention relates to an inhibitor of asparagine synthase for use for the treatment of a disorder characterized by renal and/or liver cyst formation and relative pharmaceutical composition.

### BACKGROUND OF THE INVENTION

Autosomal Dominant Polycystic Kidney Disease (ADPKD) is a monogenic disorder, caused by loss-of-function mutations in either the PKD1 (in 85% of cases) or PKD2 (in the remaining 15%) genes^{1,3,4}. The main clinical manifestation is bilateral renal cysts formation and expansion which gradually causes loss of renal function. Among additional manifestations are hypertension, liver and pancreatic cysts, cardiovascular complications such as aneurisms. The two proteins encoded by these genes, Polycystin-1 (PC-1) and Polycystin-2 (PC-2), are assembled into a functional complex at primary cilia, whose activity is defective in the disease. Additionally, PC-1 can be cleaved at several proteolytic sites⁵ resulting in products that can translocate either into the nucleus⁶, or into mitochondria⁷ or be localized at Mitochondrial-Associated-Membrane Contacts (MAMs)⁸. Cysts are epithelial outpouches of clonal origin increasing in number and size along the life of affected individuals. Inheriting one mutant allele is not sufficient for cysts to arise, but a second event causing the function of the Polycystins to drop below a critical threshold of activity is required³. Loss-of heterozygosity has been reported in a subset of cysts suggesting that this might be one of the mechanisms⁹.

Together with the deregulation of several signaling cascades, ADPKD exhibits significant metabolic alterations^{10,11,12}. Among these, defective glucose metabolism was shown to be a feature of ADPKD^{11,12} in a process resembling the Warburg effect observed in cancer. However, an overview of metabolic alterations and their interconnections is still lacking. Metabolic profiling was carried out in non-orthologous models of the disease (i.e. cystogenesis caused by mutations in other genes)_{13,14}, while a single study has attempted at profiling metabolites in the kidneys of a Pkd1 orthologous mouse model¹⁵ reporting only a minimal metabolic change in murine kidneys derived from a ubiquitous, inducible inactivation of the Pkd1 gene.

ADPKD treatment was for long time limited to interventions against secondary symptoms, such as hypertension treatment, dietary protein and sodium restriction, medication for pain relief and statin. To this end, angiotensin-converting enzyme inhibitor (ACEI) is recommended to be used as initial antihypertensive treatment. Tolvaptan, is today the only authorized specific drug for ADPKD treatment, it is an antagonist of the type 2 receptor of the hormone Vasopressin. This treatment delays the increase in kidney volume and slows the decline in renal function. Clinical studies have shown adverse events related to aquaresis (excessive thirst and urination volume) and hepatic toxicity with plasma aminotransferase elevations¹⁶, incorporated by reference).

mTOR inhibitors have been shown to have benefic effects on the disease progression in rat and murine models of PKD. However the results of the clinical studies mTOR inhibitors (Sirolimus and everolimus) showed moderated or no clinical efficacy in slowing disease progression of ADPKD patients^{17,18} For review¹⁹, (all incorporated by reference). Combination therapy of angiotensin receptor blockers (ARB) and ACEI was tested in two clinical trials to see if this could slow disease progression. The combination of ACEI and ARB was not giving any benefit in comparison to the treatment with ACEI alone^{20,21}. For review¹⁹.

Somatostatin, a peptide inhibitory hormone that signals via somatostatin receptors to inhibit the generation of intracellular cAMP. Somatostatin analogs (octreotide, lanreotide, pasireotide) have been tested in several clinical studies reveling beneficial effects which are attenuated after 2 years of treatment of ADPKD patients.

Several molecules are currently being tested in ongoing studies in ADPKD patients: tyrosine kinase inhibitor (botsutinib, tesevatinib); nicinamide, a pan-sirtuin inhibitor (review¹⁹; metformin, an inhibitor of cyclic AMP generation and activator of AMP-activated protein kinase (AMPK)²² and 2DG, an inhibitor of glycolysis²³, review²². Preclinical studies are ongoing using: methylprednisolone, urinary alkalinization, taxol, lovastatin, epidermal growth factor receptor tyrosine kinase inhibitors, peroxisome proliferator-activated receptor agonists, cyclin-dependent kinase inhibitors, and MAPK inhibitors.

Like in all other chronic Kidney Diseases, when end stage renal disease is reached, patients have the options of renal replacement therapies such as dialysis or transplant. Therefore, there is still the need for effective, specific and well tolerated treatment strategies for disorders characterized by cyst formation in the kidney and/or liver.

### SUMMARY OF THE INVENTION

In the present invention a comprehensive metabolomics characterisation of cells and renal tissues from a mouse model carrying the kidney-specific inactivation of the Pkd1 gene was performed. Present data indicate a broad metabolic rewiring that involves several pathways including central carbon metabolism and glutamine utilisation. The inventors show that loss of Pkd1 leads to profound metabolic changes that affect glycolysis, mitochondrial metabolism, and fatty acid synthesis (FAS). In particular, the inventors found that Pkd1-mutant cells preferentially use glutamine to fuel the TCA cycle, and to sustain FAS. Interfering with either glutamine uptake or FAS retards cell growth and survival. The inventors also found that the glutamine is diverted to asparagine via asparagine synthetase (ASNS). Notably, the silencing of Asns was lethal in Pkd1-mutant cells when combined with glucose deprivation, opening novel therapeutic perspectives for ADPKD.

Then a broad and coordinated metabolic reprogramming in PKD is shown in the present invention highlighting the identification of potential novel therapeutic strategies for the treatment of a disorder characterized by renal and/or liver cyst formation.

Then the present invention provides an inhibitor of asparagine synthase for use for the treatment of a disorder characterized by renal and/or liver cyst formation.

Preferably the inhibitor is selected from the group consisting of: a small molecule, an organic inhibitor, an oligonucleotide, an antibody.

Still preferably the inhibitor is a siRNA or antisense oligonucleotide (ASO) or an inhibitor of ATF4.

Preferably the oligonucleotide is a locked nucleic acids (LNAs). LNAs are chemically modified nucleotides with a ribose containing a methylene bridge between the 2′-oxygen and the 4′-carbon of the ribose. LNA modifications improve significantly the ASO hybridization affinity towards mRNA target, due to the important increase in the thermal stability of the DNA/RNA heteroduplexes²⁴. In addition, LNAs avoid nuclease degradation. As their ribose 2′-O position are modified, LNAs are not recruiting RNase H of the target mRNA²⁴.

Preferably the oligonucleotide is a modified oligonucleotide, preferably a locked nucleic acid (LNA)-modified oligonucleotide or a 2′-O-Methyl-modified oligonucleotide. LNA is generally considered to be an RNA mimic in which the ribose sugar moiety is locked by an oxymethylene bridge connecting the C(2′)- and C(4′)-atoms which conformationally restricts LNA monomers into an N-type sugar puckering. LNA is a molecule which contains at least one nucleotide bearing the LNA modification. Preferably the LNA contains, at least 2, at least 3, at least 4, at least 5, at least 10, at least 15, at least 20 nucleotides bearing the LNA modification.

2′-O-Methyl-modified oligonucleotide is a molecule which contains at least one nucleotide bearing the 2′-O-Methyl modification. Preferably the 2′-O-Methyl-modified oligonucleotide contains, at least 2, at least 3, at least 4, at least 5, at least 10, at least 15, at least 20 nucleotides bearing the 2′-O-Methyl modification.

More preferably the inhibitor is used in combination with an inhibitor of glycolysis. Preferably the inhibitor of glycolysis is selected from the group consisting of: a glucose analogue, preferably selected from the group comprising 2DG, SB-204990, 3-bromopyruvate (3-BrPA), 3-BrOP, 5-thioglucose, mannose, galactose, gulose, a 2DG having a fluorine in place of a hydrogen at any position on the glucose ring, a 2DG having an amino group in place of a hydroxyl group at any position on the glucose ring other than the 6 position, 2-F-mannose, 2-mannosamine, 2-deoxygalactose, 2-F-deoxygalactose, a di, tri, and other oligosaccharides that contain one or more of the preceding 2DG analogs; a small-molecule inhibitor of Hesokinase (HK), Phosphofructokinase, Glucose-6-phosphate Dehydrogenase (G6PD), Transketolase-like enzyme 1 (TKTL1), Glyceraldehyde-3-phosphate Dehydrogenase (GAPDH), Pyruvate kinase, Lactate Dehydrogenase (LDH), said small-molecules being preferably selected from the group comprising: 3-BrPA, 2DG, 6-aminonicotinamide (6-AN), oxythiamine, Arsenic, Dichloroacetic acid (DCA), N-Hydroxyindoles (NHI).

Preferably the disorder characterized by renal and/or liver cyst formation is selected from the group consisting of: autosomal dominant polycystic kidney disease, nephornophthisis (NPHP), in particular affecting children and adolescents, Oral Facial Digital Syndrome (OFD1), Bardet Biedle Syndrome (BBS), Polycystic Liver Disease, Autosomal Dominant Polycystic Liver Disease (ADPLD) condition. These diseases are “ciliopathies” which manifest in several different organs and invariably present with cysts in the kidney. The molecular basis of cyst formation are supposed to be shared by all these diseases.

The present invention also provides a pharmaceutical composition comprising an inhibitor of asparagine synthase as defined above and pharmaceutically acceptable excipients for use for the treatment of a disorder characterized by a by renal and/or liver cyst formation. Preferably the composition further comprises an inhibitor of glycolysis or further comprises at least a further agent, said agent being preferably selected from the group consisting of: an inhibitor of fatty acid synthase and/or an inhibitor of Ascorbate and Aldarate Metabolism and/or an inhibitor of Nicotinate and Nicotinamide Metabolism and/or an inhibitor of Primary Bile Acid Metabolism and/or an inhibitor of Purine and Pyrimidine metabolism and/or an inhibitor of Fructose, Mannose and Galactose Metabolism and/or an inhibitor of Pentose Phosphate Pathway and/or an inhibitor of Glutathione and Polyamine Metabolism and/or an inhibitor of Methionine, Cysteine, SAM and Taurine Metabolism and/or an inhibitor of Tryptophan, Phenylalanine and Tyrosine Metabolism and/or an inhibitor of N-terminal acetylation of aminoacids.

More preferably the pharmaceutical composition further comprises a therapeutic agent, preferably said therapeutic agent is selected from the group consisting of: a renin-angiotensin-aldosterone system (RAAS) inhibitor, preferably an angiotensin-converting enzyme (ACE) inhibitor or an angiotensin II receptor blocker (ARB), an antagonist of the type 2 receptor of the hormone Vasopressin, a mTOR inhibitor, a somatostatin analog, a tyrosine kinase inhibitor, a sirtuin inhibitor, an epidermal growth factor receptor tyrosine kinase inhibitor, a peroxisome proliferator-activated receptor agonist, a cyclin-dependent kinase inhibitor, and a MAPK inhibitor. Preferably, the therapeutic agent is Tolvaptan, nicinamide, metformin, methylprednisolone, an urinary alkalinization, taxol, lovastatin or any agent disclosed in Chapman et al. 2018¹⁶. Literature review current through: July 2018. Available from: http colon forward slash forward slash www dot uptodate dot com forward slash contents/course-and-treatment-of-autosomal-dominant-polycystic-kidney-disease?source=search_result, incorporated by reference.

Preferably the treatment of a disorder characterized by a by renal and/or liver cyst formation is ADPKD, preferably ADPKD caused by mutation in PKD1 gene.

The present invention also provides an inhibitor of fatty acid synthase for use in the treatment of a disorder characterized by renal and/or liver cyst formation. In the present invention an ASNS inhibitor act on the activity and/or the expression of ASNS. ASNS activity measured by any known method in the art, in particular Kinetic studies by HPLC-based end-point assay in which recombinant ASNS incubated with substrates at various inhibitor concentrations as per Gutierrez et al. 2006²⁵. The level of expression of ASNS may be measured by RT-PCR and Western blot.

Based on the crystal structure of ASNS from Escherichia coli²⁶ the human enzyme is built from two domains containing each one a catalytic site. The N-terminal site catalyzes the conversion of glutamine into glutamic acid and ammonia, and the C-terminal site is involved in the generation of the reactive intermediate β-aspartyl-AMP (βAspAMP)²⁵. The ASNS inhibitor of the present invention may be a binding molecule that binds to the catalytic sites of ASNS, thereby inhibiting its activity. For instance the stable analog of the transition state for asparagine formation can be used (see below the example of Adenylated sulfoximine). The inhibitor may be as described in Zhu et al.⁵⁴, incorporated by reference.

The ASNS inhibitor of the present invention may be an allosteric inhibitor, i.e. a mutant glutamine derivative or glutamine processing metabolite, or glutamate derivative able to bind to ASNS inhibiting the aminotransferase activity of ASNS²⁷.

The ASNS inhibitor of the present invention may act upstream of ASNS (for example targeting ATF4, an activator of the transcription of ASNS).

The ASNS inhibitor of the present invention may also be a downstream molecule of the pathway such as inhibitors of integrated stress response proteins, that regulate amino acid homeostasis or the cellular response to ASNS upregulation²⁸.

The ASNS inhibitor of the present invention may also act on the substrates, acting on the availability of aspartate and glutamine or their transport^{25,26}.

The ASNS inhibitor of the present invention may be an organic inhibitor of ASNS such as adenylated sulfoximine which inhibits hASNS with nanomolar potency²⁵. An analog of this compound, an amino sulfoximine having no net charge at cellular pH, is also provided as hASNS inhibitor^{25,29}.

Other organic inhibitors of the enzyme L-asparagine synthetase are: guanidinosuccinic acid; oxaloacetic acid; L-cysteinesulfinic acid; diethyl aminomalonate; dipeptides containing L-aspartic acid (L-aspartylglycine, L-aspartyl-L-leucine, L-aspartyl-L-phenylalanine, L-aspartyl-L-proline, L-α-aspartyl-L-serine and L-α-aspartyl-L-valine); N-o-nitrophenylsulfenyl-L-aspartic acid; N-onitrophenylsulfenyl-L-glutamine; S-adenosyl-L-methionine; L-homoserine-3 adenylate; palmitic acid; lauric acid; and ethacrynic acid. ASNS expression can be inhibited using siRNA and single-stranded antisense oligonucleotides (ASOs) inhibiting ASNS. (ASOs) are short, synthetic DNA molecules that bind to target RNA and catalyse downstream actions.

The level of expression of ASNS may be measured by RT-PCR, Western blot, immunohistochemistry, ELISA assays, luciferase reporter assays (measuring induction of a reporter gene (e.g., luciferase) that is fused to the ASNS promoter (responding to ATF4).

### ATF4

An inhibitor of ATF4 activity may inhibit the transcriptional activity of ATF4 and/or increase its degradation. The inhibitor may also reduce ATF4 translation by inhibiting upstream eIF2α kinases. The inhibitor can also acts on the downstream pathways such as the transcriptional activity or nuclear translocation of ATF4 and it binding to the promoter of enzymes involved in amino acid biosynthesis such as ASNS as well as ER-associated degradation and autophagy.

The molecules reducing or inhibiting ATF4 activity include: Thimerosal; Gambogic Acid; Anthothecol; Disulfiram; Pyrithione Zinc; Thiram; Tomatine; Dihydrogambogic Acid; Trifluoperazine Hydrochloride; Alexidine Hydrochloride; Phenylmercuric Acetate; Pristimerin; Aklavine Hydrochloride; 6,3′-Dimethoxyflavone; Tetrachloroisophthalonitrile; Actinomycin D; Cedrelone; Pyrromycin; Mitoxanthrone Hydrochloride; Tyrothricin; Selinidin; Gentian Violet; Clofoctol; Aminacrine; Penicillic Acid; Byssochlamic Acid; Hieracin; Atranorin; Dihydrojasmonic Acid; Deltaline; Azaserine; Sodium Fluoroacetate; Thalidomide; Neomycin Sulfate; Camptothecin; Trimedlure (5-Cl Isomer Present); Chlorguanide Hydrochloride; Benzo[a]pyrene; Hycanthone; Methotrexate; Dihydrorotenone; Galanthamine Hydrobromide; Ipraflavone; 5,7-Dichlorokynurenic Acid; Haematoporphyrin Dihydrochloride; Osthol; 1r,2s-Phenylpropylamine; 2,4-Dinitrophenol; Bromopride; Isorotenone; Lycorine; Halcinonide; 7-Desacetoxy-6,7-Dehydrogedunin; 6-Aminonicotinamide; óalpha-methylprednisolone acetate; Teniposide; 1-Methylxanthine; Mercaptopurine;

Tripelennamine Citrate; 9-Amino-1,2,3,4-Tetrahydroacridine Hydrochloride; Beta-Dihydrorotenone; Acivicin; Hydroxytacrine Maleate; or Cetylpyridinium Chloride and are part of the present invention.

ATF4 expression can be inhibited using siRNA and single-stranded antisense oligonucleotides (ASOs) inhibiting ASNS. (ASOs) are short, synthetic DNA molecules that bind to target RNA and catalyse downstream actions.

ATF4 activity and translation may be measured by any known methods for instance by HPLC-based kinetic studies and by assessing the level of activity of an ATF4 target gene such as Asns, heme oxygenase 1, stanniocalcin 2, osteocalcin, gadd153/CHOP, and TRB3.

The level of ATF4 may be measured by RT-PCR, Western blot, immunohistochemistry, ELISA assays, luciferase reporter assays (measuring induction of a reporter gene (e.g., luciferase) that is fused to a promoter responding to the ATF4 gene.

### Glycolysis

For the purpose of glycolysis inhibition, in a preferred embodiment, at least one glucose analogue is used, preferably selected from the group comprising 2DG, SB-204990, 3-bromopyruvate (3-BrPA), 3-BrOP, 5-thioglucose, mannose, galactose, gulose, a 2DG having a fluorine in place of a hydrogen at any position on the glucose ring, a 2DG having an amino group in place of a hydroxyl group at any position on the glucose ring other than the 6 position, 2-F-mannose, 2-mannosamine, 2-deoxygalactose, 2-F-deoxygalactose, and di, tri, and other oligosaccharides that contain one or more of the preceding 2DG analogs In a further embodiment, said glucose analogues are selected from the following group:

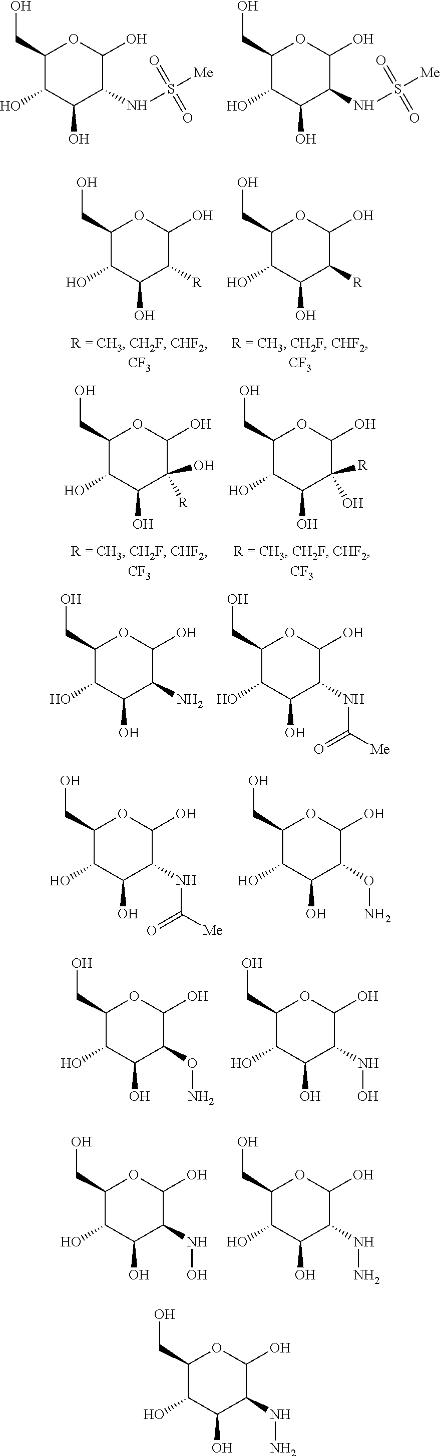

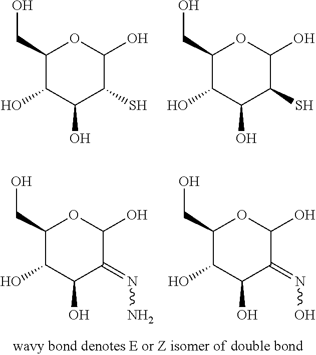 [SMILES: OC[C@H]1OC(O)[C@H](S)[C@@H](O)[C@@H]1O ; OC[C@H]1OC(O)[C@@H](S)[C@@H](O)[C@@H]1O ; NN=C1C(O)O[C@H](CO)[C@@H](O)[C@@H]1O ; OC[C@H]1OC(O)C(=NO)[C@@H](O)[C@@H]1O]

In an alternative embodiment, said inhibitor of glycolysis is selected from small-molecule inhibitors of Hexokinase (HK), Phosphofructokinase, Glucose-6-phosphate Dehydrogenase (G6PD), Transketolase-like enzyme 1 (TKTL1), Glyceraldehyde-3-phosphate Dehydrogenase (GAPDH), Pyruvate kinase, Lactate Dehydrogenase (LDH). Said small-molecules are preferably selected from the group comprising 3-BrPA, 2DG, 6-aminonicotinamide (6-AN), oxythiamine, Arsenic, Dichloroacetic acid (DCA), N-Hydroxyindoles (NHI). In a further embodiment, the present invention is related to a pharmaceutical composition comprising at least one glycolysis inhibitor selected among glucose analogues or in the group comprising small-molecule inhibitors of Hesokinase (HK), Phosphofructokinase, Glucose-6-phosphate Dehydrogenase (G6PD), Transketolase-like enzyme 1 (TKTL1), Glyceraldehyde-3-phosphate Dehydrogenase (GAPDH), Pyruvate kinase, Lactate Dehydrogenase (LDH).

### FAS Inhibition

Inhibitor of fatty acid synthase (FAS) may inhibit the activity and/or the expression of FAS.

FASN catalyzes the synthesis of palmitate from acetyl-CoA and malonyl-CoA, Palmitate is then converted in other fatty acids.

The inhibitor may bind to the catalytic sites of the FAS enzyme and/or may reduce the availability of the substrates acetyl-CoA and malonyl-CoA.

Fas activity can be inhibited using small molecules like Orlistat, Cerrulenin, C75, C93. Natural inhibitors of Fas activity such as Phenolics, Terpenoids, Steroidal sapogenin and the soy peptides KNPQLR, EITPEKNPQLR and RKQEEDEDEEQQRE can also be used³⁰ (incorporated by reference).

Further the inhibitor can inhibit the FAS coding gene FASN expression as a siRNA and single-stranded antisense oligonucleotides (ASOs) inhibiting FASN.

The present invention also provides modulators of Ascorbate and Aldarate Metabolism and/or Nicotinate and Nicotinamide Metabolism and/or Purine and Pyrimidine metabolism and/or Primary Bile Acid Metabolism and/or Fructose, Mannose and Galactose Metabolism and/or Pentose Phosphate Pathway and/or Phenylalanine and Tyrosine Metabolism and/or Glutathione and Polyamine Metabolism and/or N-terminal acetylation of aminoacids and/or Methionine, Cysteine, SAM and Taurine Metabolism for use in the treatment of autosomal dominant polycystic kidney disease.

Interestingly, 4-hydroxyphenylpyruvate, a keto acid that is involved in the tyrosine catabolism pathway, was strikingly increased (239.91 fold) in the kidneys from KspCre:Pkd1^{flox/flox} mice compared to the wild-type. Accumulation of this metabolite is observed in Tyrosinemia type III caused by the deficiency of 4-hydroxyphenylpyruvate dioxygenase (HPD), the second enzyme involved in the tyrosine catabolic pathway³¹.

### Modulators of Glutathione and Polyamine Metabolism

The present invention revealed an alteration of Glutahione and Polyamine metabolism, in particular the reduced glutathione was increased by 38.88 fold whereas the oxidized form was reduced by 0.54 fold in the kidneys from KspCre:Pkd1^{flox/flox} compared to the ones from control mice. This data potentially reflects increased anti-oxidant capacity.

### Modulators of N-Terminal Acetylation of Aminoacids

The present data have shown that amino acids (Glycine, Serine, Threonine, Alanine, Aspartate, Asparagine, Glutamate, Histidine, Lysine, Phenylalanine, Tryptophane, Leucine, Isoleucine, Valine, Methionine, Arginine and Taurine) are less abundant in the cystic compared to the wild-type kidney. On the opposite, N-Acetylated amino acids (N-acetylarginine, N-acetylserine, N-acetylthreonine, N-acetylalanine, N-acetylasparagine, N-acetylglutamate, N-acetylglutamine, N-acetylhistidine, N-acetyltyrosine, N-acetylleucine, N-acetylisoleucine, N-acetylvaline, N-acetyltaurine) are increased in the cystic in comparison to the controls kidneys. This may indicate a change in amino acid homeostasis as well as in enzymes and pathways involved in the N-terminal acetylation of aminoacids.

### Modulators of Methionine, Cysteine, SAM and Taurine Metabolism

In the present invention it was found a decrease in the amino acid taurine in the Pkd1 mutant kidneys as compared to controls and a concomitant increase in N-terminal acetylated Taurine. Present data have shown a decrease in: methionine, N-acetylmethionine, S-adenosylhomocysteine, cysteine, S-methylcysteine, cysteine sulfinic acid, taurocyamine, 2-hydroxybutyrate/2-hydroxyisobutyrate. Present data have shown an increase in: N-formylmethionine and cystathionine.

### Modulators of Nicotinate and Nicotinamide Metabolism

Treatment with nicotinamide delayed cyst growth in Pkd1 knockout mouse embryonic kidneys, Pkd1 conditional knockout postnatal kidneys, and Pkd1 hypomorphic kidneys. Present data in the Pkd1 mutant kidneys as compared to controls have shown a decrease in: nicotinamide, nicotinamide ribonucleotide, nicotinamide adenine dinucleotide (NAD⁺), nicotinamide adenine dinucleotide reduced (NADH) and adenosine 5′-diphosphoribose (ADP-ribose) whilst an increase in: nicotinamide riboside, nicotinamide N-oxide, 1-methylnicotinamide, trigonelline (N′-methylnicotinate) and N1-Methyl-2-pyridone-5-carboxamide.

The present invention will be illustrated by means of non-limiting examples in reference to the following figures and abbreviations.

### Abbreviations

α-KG, alpha-ketoglutarate
ASNS, asparagine synthetase
1,3BPG, 1,3-bisphosphoglycerate
BUN, blood urea nitrogen
DCA, dicarboxylic acids
ECAR extracellular acidification rate
FA-Carn, fatty acyl-carnitine
FAS, fatty acid synthesis
FAO, fatty acid oxidation
FI, Fold Induction
FFA, free fatty acids
F6P, fructose 6-phosphate
F1,6BP, fructose 1,6-bisphosphate
G6P, glucose 6-phosphate
GAP, glyceraldehyde 3-phosphate
Gln, glutamine
Glu, glucose
LCFA, long chain fatty acids
LC-MS, liquid chromatography coupled to mass spectrometry
MalCoA, malonyl-CoA
Mal, Malonate
MS, mass spectometry
MUFA, monounsaturated fatty acids
OAA, oxoloacetate
OCR, oxygen consumption rate
OXPhos, oxidative phosphorylation
PEP, phosphoenolpyruvate
6PG, 6-phosphogluconate
2PG, 2-phosphoglycerate
3PG, 3-phosphoglycerate
PPP, pentose phosphate pathway
PUFA, polyunsaturated fatty acid
R5P, ribose 5-phosphate
Ri5P, ribulose 5-phosphate
SFA, saturated fatty acids
SPF, specific pathogen free
S7P, sedoheptulose 7-phosphate
TCA, tricarboxylic acid
X5P, xylulose 5-phosphate

### BRIEF DESCRIPTION OF DRAWINGS

FIGS. 1A-F. Global metabolomic profiling reveals defective TCA cycle in PKD. (1A) Study design of the experiment performed on kidneys from Ksp-cre;Pkd1^{-/flox} at P4. 4 litters each containing 2 cystic (red) Ksp-cre;Pkd1^{-/flox} and 2 control littermates Ksp-Cre; Pkd1 ^{flox/+} or Pkd1^{flox/+} (blue, used interchangeably) were collected. Samples were processed for analysis by Ultrahigh Performance Liquid Chromatography-Tandem Mass Spectrometry. (1B) Dot plot view showing percentage of kidney/body weight in the cystic and control kidneys. (1C) PCA applied to the identified metabolites, shows a good separation between cystic versus control kidneys. (1D) HCA shows good clustering between the groups of cystic (bottom) and control kidneys samples (top). (1E) Significant metabolites were colour-coded according to the pathway classification. Scheme of the Glycolysis (GLY), Pentose Phosphate Pathway (PPP), Tricarboxylic acid (TCA) cycle, Fatty acid biosynthesis (FAS), Fatty acids oxidation (FAO) in cystic versus control kidneys. Colour corresponds to the fold changes between cystic and control kidneys, orange-red are metabolites more abundant in cystic compared to the control kidneys, whereas green labelled ones correspond to the less abundant metabolites in cystic kidneys compared to controls. (1F) Box and whiskers view of the levels of TCA intermediates citrate, aconitate and α-ketoglarate (α-KG) were assessed by LC-MS, they were significantly more abundant in cystic compared to the control kidneys. n=8 independent biological replicates. All dot blots are shown as mean and error bars as SEM, n.s., not significant (p-value>0.05), *p-value<0.05; **p-value<0.001; ***p-value<0.0001; *p-value<0.00001, t-test for (1B, 1E and 1F).

FIGS. 2A-G. Impaired respiration and usage of glucose in the TCA cycle in Pkd1^{-/-} cells.

(2A and 2B) Analysis of extracellular acidification rate (ECAR) outputs of Pkd1^{-/-} compared to Pkd1^{+/+} cells subject to glycolysis tests in response to glucose, oligomycin and 2-DG. (2B) Dot plots showing means with 28 to 39 replicate wells per group for glycolysis, glycolytic capacity and glycolytic reserve. Glycolysis was calculated on the 6^{th} measurement time (after subtraction of 3^{rd} measurement); the glycolytic reserve was measured on the 9^{th} measurement time (after subtraction of 6^{th} measurement); the glycolytic capacity was calculated on the 9^{th} measurement time (after subtraction of the 3^{rd} measurement). (2C and 2D) Representative analysis of OCR measurement in Pkd1^{+/+} and Pkd1^{-/-} cells in basal conditions and after sequential addition of oligomycin, 7 Carbonyl cyanide-4 trifluoromethoxy phenylhydrazone (FCCP) and antimycin/rotenone (A/R). (2D) Dot plots showing means with 6 replicate wells per group for: MRR calculated from the 10^{th} time subtracted from the 13^{th} time; OCR/ECAR ratio from basal measurement, 2^{nd} time point. (2A-2D) representative of three independent experiments e The scheme illustrates the fate of glucose C-atoms in glycolysis and Krebs cycle intermediates. Cells were incubated in glucose-free DMEM supplemented with 25 mM ¹³C₆-glucose for 24 hrs. The uniformly labelled glucose (M+6 yellow) leads to formation of M+3 lactate (dark brown) or M+2 TCA cycle intermediates after its first round (M+2 light brown). (2F) Percentage of isotopologue distribution of intracellular and extracellular glucose and lactate shows that glucose (M+6) is more consumed and more converted into lactate (M+3) in Pkd1^{-/-} cells in comparison to control cells (see methods for calculations). (2G) Percentage of isotopologue distribution of intracellular intermediates of the TCA: α-KG, succinate, fumarate and malate shows that the molecules coming from glucose (M+2) are decreased in Pkd1^{-/-} MEFs compared to the control cells. Graphs (2F and 2G) are means in percentages relative to control cells of six technical replicates from one experiment. Mean+/−SEM were indicated, n.s., not significant (p-value≥0.05), *p-value<0.05; **p-value<0.001; ***p-value<0.0001; ****p-value<0.00001, t-test.

FIGS. 3A-H. Metabolic rearrangement in bioenergetics pathways and glutaminolysis rewiring in Pkd1^{-/-} cells.

(3A) The scheme illustrates the fate of glutamine C atoms in Krebs cycle intermediates (Oxidative). Cells were incubated in glutamine-free DMEM supplemented with 4 mM ¹³C₅-¹⁵N₂glutamine for 24 hrs. (3B) Quantification of the intracellular labelled ¹³C₅-¹⁵N₂glutamine showing that Pkd1^{-/-} cells have a higher uptake compared to the controls. (3C) Isotopologue distribution of intracellular α-KG shows that pools containing five ¹³C (M+5), coming from labelled glutaminolysis, were all significantly higher in Pkd1^{-/-} as compared with Pkd1^{+/+} cells. (3D) Isotopologue distribution of intracellular succinate, fumarate and malate shows that pools containing four ¹³C (M+4), coming from labelled glutaminolysis, were all significantly higher in Pkd1^{-/-} as compared with Pkd1^{+/+} cells. Graphs are means in percentages relative to control cells of six technical replicates from one experiment. (3E) Representative graph showing the results of the XFMitoFuel Flex Test analysis measuring OCR in response to either glucose or glutamine from 8 technical replicates per well of two independent experiments. Data show a reduced oxidation of glucose in Pkd1^{-/-} cells and an increased dependency on glutamine compared to wild-type cells. (3F) Cells were grown under either glucose or glutamine or both limiting conditions for 24 and 48 hrs after an overnight 0.5% serum starvation. Representative images showing the cellular morphology of Pkd1^{-/-} compared to Pkd1^{+/+} cells in starved and non-starved conditions. (3G) Viability of Pkd1^{+/+} and Pkd1^{-/-} cells, expressed as percentage cell count compared to time 0, after 24 hrs of incubation in limiting conditions of glucose, glutamine, or both. (3H) Percentage of apoptotic cells assayed by Tunel assay, showing significant higher percentage of apoptotic cells in limiting conditions. Graphs (3F to 3H) are representative of at least three independent experiments, data are means from at least three technical replicates. Mean+/−SEM were indicated, n.s., not significant (p-value≥0.05), *p-value<0.05; **p-value<0.001; ***p-value<0.0001; ****p-value<0.00001. t-test (3B, 3C and 3D). ANOVA followed by Bonferroni for (3G and 3H).

FIGS. 4A-I. Glutamine usage is interlinked with asparagine synthase (ASNS) in PKD.

(4A) Schematic overview of the conversion of glutamine into glutamate by transaminating aspartate into asparagine by ASNS. (4B) Labelled ¹⁵N-asparagine is significantly increased in the Pkd1 mutant MEFs (left), total levels of asparagine are increased compared to the control cells and decreasing levels of aspartate (M+0), expressed as percentage relative to controls. (4C) Left: Isotopologue distribution of intracellular asparagine showing that the pool coming from glutamine (¹⁵N₁ and ¹⁵N₂) is significantly decreased in siAsns compared to the mock Pkd1^{-/-} and control cells. Right: Total α-KG was significantly decreased in SiAsns compared to the mock Pkd1^{-/-} and control cells in the ¹⁵N₂ glutamine labeling. (4D) Left: Total asparagine was significantly decreased in SiAsns compared to the mock Pkd1^{-/-} cells. Right: α-KG (M+5) in the ¹³C₅-glutamine labeling was significantly decreased in SiAsns compared to the mock Pkd1^{-/-} and control cells. Data are means from six technical replicates from one experiment. (4E) Representative graph showing the percentage of cell count compared to the respective mock controls. Silencing Asns, deprivation of glucose or both treatments in Pkd1^{+/+} resulted in no significant difference, whilst each condition resulted in a significant reduction in cell number with an additive effect of Asns silencing with glucose starvation in Pkd1^{-/-} cells. Data are means from three technical replicates from two independent experiments. Mean+/−SEM were indicated, n.s., not significant (p-value≥0.05), *p-value<0.05; **p-value<0.001; ***p-value<0.0001; ****p-value<0.00001, t-test for (4A and 4C). ANOVA followed by Bonferroni for (4D, 4E and 4F). (4F) Metabolic tracing with ¹⁵N₂-glutamine showed that silencing of Asns reduced the total levels of α-KG. (4G) The silencing of Asns reduced the intracellular levels of total asparagine in Pkd1^{-/-} cells. (4H) Metabolic tracing with ¹³C₅-glutamine showed a significant reduction in the glutamine-derived α-KG (M+5). (4I) The down-regulation of Asns resulted in reduced cell numbers in Pkd1^{-/-} cells, but not in controls. When siAsns: Pkd1^{-/-} cells were subject to glucose starvation there was a further decrease in cell numbers compared to controls treated under the same conditions.

FIGS. 5A-I: Glutaminolysis and Fatty acids Synthase dependence of Pkd1^{-/-} cells. (5A) The scheme illustrates the fate of glutamine C atoms in TCA cycle intermediates and fatty acid biosynthesis. (5B) Quantification of intracellular citrate (M+5) labelled by glutamine was significantly higher in Pkd1^{-/-} as compared with Pkd1^{+/+} cells. (5C) Incorporation of glutamine carbons into intracellular palmitate (M+2) was significantly higher in Pkd1^{-/-} as compared with Pkd1^{+/+} cells. Data are means from six technical replicates from one independent experiments. (5D) Dot plots showing means fold change, SEM, mRNA levels of Fasn in Pkd1^{-/-} compared to mock (transfection reagent alone) Pkd1^{+/+} cells and SiFasnPkd1^{-/-} compared to mock Pkd1^{-/-} normalised to Hprt. Three technical replicates of at least 3 independent experiments. (5E) Dot plots showing means fold change, SEM, mRNA Fasn levels measured in control compared to cystic kidneys normalised to Hprt. n=8, two technical replicates of at least 2 independent experiments. (5F) Representative images showing cellular morphology of SiFasn in Pkd1^{+/+} and Pkd1^{-/-} and mock controls. (5G) Representative graph of percentage Ki67 positive cells for mock and SiF asn for Pkd1^{+/+} and Pkd1^{-/-} cells after 48 hrs of transient silencing. Data from three technical replicates of at least two independent experiments. (5H) Representative graphs showing percentage of apoptotic cells after 48 hrs of transfection, data from three technical replicates from three independent experiments. (5I) Lipidomic profiling of cystic versus control kidneys showed altered percentage of diacylglycerol (DAG: 38.4), three different species of triacylglycerol (TAG: 50, TAG: 52 TAG: 54) and sterol esters (SE: 43) from total lipid content, n=6 Scale bar is 100 μm. Mean+/−SEM were indicated, n.s., not significant (p>0.05), *p-value<0.05; **p-value<0.001; ***p-value<0.0001; ****p-value<0.00001. Mean+/−SEM were indicated, n.s., not significant (p-value≥0.05), *p-value<0.05; **p-value<0.001; ***p-value<0.0001; ****p-value<0.00001, 1-test for (5B, 5C and 5E), ANOVA followed by Bonferroni for (5D, 5G, 5H), ANOVA for 5I.

FIGS. 6A-D: Global metabolic changes from in silico simulation and transcriptional profiling in PKD.

(6A) Analysis of metabolic rearrangement in bioenergetic pathways resulting from in silico simulations. The Differential Abundance (DA) score captures the average, gross changes of al metabolic fluxes in a pathway. A score of −1 indicates that all the simulated metabolic fluxes in the pathway decrease, while a score of 1 indicates that all in silico fluxes increase upon comparing the simulations of increased of glucose uptake with the computations of wild-type conditions. (6B) Representation of the results of metabolic changes in the DFA in silico simulations upon increased glucose uptake. Data show that equilibrium is reached when upregulation of GLY, PPP, glutaminolysis and FAS is achieved, while a reduction of OXPHOS and FAO. (6C) Identification of genes differentially expressed in Pkd1^{V/V} kidneys versus controls in GLY, PPP, TCA/OXPHOS, FAS and FAO. D-val represents the output of SAM algorithm launched with the parameter delta set to 1.0. All genes in the panel were differentially expressed between Pkd1^{V/V} animal model and wild type model at P10 with a FDR equal to 0.1. Up-regulated genes are shown in red, while down-regulated ones are reported in blue. (6D) Identification of genes differentially expressed in GLY, PPP, TCA/OXPHOS, FAS and FAO in human PKD1 microarrays datasets. D-val represents the output of SAM algorithm launched with the parameter delta set to 2.4. All genes in the panel were differentially expressed between cystic (small, medium and large cysts) versus control tissues (minimal cystic and normal renal cortical tissues) with a FDR equal to zero. n=4 for Pkd1^{V/V} animal model, n=8 (normal and minimal cyst) and n=13 for large, medium, small cyst for human PKD1 microarrays.

FIGS. 7A-E. (7A) Representative immunohistochemistry of cystic kidneys and control kidneys (P4) against CD45, Collagen I (COLI), Collagen IV (COLIV), respective markers of inflammation and fibrosis, showing no inflammation and only very mild fibrosis. Panels on the left and on the right show higher magnification. Bar represents 100 μm. (7B) Blood urea nitrogen (BUN) in mg/dL showing higher levels of BUN in cystic mice when compared to littermate controls collected at P4, P8, P10 and P12. (7C) Epo and Vegf mRNA expression was assessed by quantitative RT-PCR, normalised to HPRT. No significant increase was detected for Epo and Vegf in the cystic kidneys when compared to the controls. (7D) Volcano plot of the 550 named metabolites profiled from mice kidneys. 384 metabolites exhibited significant changes (adjusted p value<0.05, absolute fold change>2) when comparing cystic kidneys with control kidneys. 171 metabolites exhibited a significant increase whilst 213 metabolites exhibited a significant decrease. Paired Student's t-tests were used to calculate statistical significance, and p values were corrected by using the Benjamini-Hochberg procedure. Differentially abundant metabolites of different categories have been individually colour coded. (7E) KEGG-Pathways Based Enrichment Analysis applied to the list of metabolites deriving from non-target global metabolomics performed on the KspCre;Pkd^{flox/-} kidneys and ranked in descending order according to the p values. It reveals 26 mouse pathways with a statistically significant (p≤0.05) enrichment score. Particularly, 8 pathways are enriched for upregulated metabolites whilst 18 are enriched for downregulated ones. Mean+/−SEM were indicated, n.s., not significant (p≥0.05), *p<0.05; **p<0.001; ***p<0.0001; ****p<0.00001. t-test for 7C and ANOVA for 7B. Data in 7D and 7E were obtained from eight independent biological replicates.

FIGS. 8A-D. (8A) Principal component analysis (PCA) applied to the metabolites identified showed good separation between Pkd1^{+/+} and Pkd1^{-/-} cells. (8B) Volcano plot of the metabolites profiled from Pkd1^{+/+} and Pkd1^{-/-} cells. 122 metabolites exhibited significant changes (adjusted p value<0.05, absolute fold change >2) when comparing Pkd1^{+/+} with Pkd1^{-/-} cells. 31 metabolites exhibited a significant increase whilst 91 metabolites exhibited a significant decrease. Paired Student's t-tests were used to calculate statistical significance, and p values were corrected by using the Benjamini-Hochberg procedure. Differentially abundant metabolites of different categories have been individually colour coded. (8C) Significant metabolites were individually colour coded according to the pathway classification. Scheme of the glycolysis, pentose phosphate pathway, fatty acid oxidation and fatty acid biosynthesis Pkd1^{+/+} and Pkd1^{-/-} cells. Colour corresponds to the fold changes between Pkd1^{-/-} and Pkd1^{+/+} cells, orange-red labelled ones correspond to the metabolites more abundant, whereas green labelled ones correspond to the metabolites less abundant in Pkd1^{+/+} compared Pkd1^{-/-} cells. Untargeted methodology is shown in the blue box whereas the targeted are shown in the green box. (8D) Levels of TCA intermediates citrate, α-KG, succinate and malate were assessed by LC-MS in Pkd1^{+/+} and Pkd1^{-/-} MEFs showing that they are significantly more abundant in the mutant cells compared to the controls. Dot plots showing means, SEM. n.s., not significant (p≥0.05), *p<0.05; **p<0.001; ***p<0.0001; ****p<0.00001. t-test for 8D and six technical replicated for 8D. All graphs represent the data of seven technical replicates of one experiment.

FIGS. 9A-C. (9A) Oxygen consumption rate (OCR) and extracellular acidification rate (ECAR) were measured using the XF Extracellular Flux Analyzer (Seahorse Bioscience) on primary mouse embryonic fibroblasts isolated from Pkd1^{+/+}+ or Pkd1^{ΔC/ΔC} embryos at E11.5 and (9B) shPkd1 (C16, C12) cells compared to scrambled controls (M3, M4). (9C) Extracellular lactate. Levels of unlabelled M+0 extracellular lactate were measured in Pkd1^{-/-} and Pkd1^{+/+} cells in the presence (+) or in the absence (−) of ¹²C-glucose. Mean+/−SEM were n.s., indicated, not significant (p≥0.05), *p<0.05; **p<0.001; ***p<0.0001; ****p<0.00001. t-test for a and b, ANOVA for c. Representative graphs of at least two independent experiments made from 33 to 38 (9A) and from 20 (9B) replicate wells. One experiment made with six technical replicates is represented in 9C.

FIGS. 10A-C. (10A) Dot plots showing means fold change, SEM, mRNA levels of Asns in Pkd1^{-/-} compared to Pkd1^{+/+} cells normalised to Hprt. n=3, three technical replicates of at least 3 independent experiments. Dot plots showing means fold change, SEM, mRNA Asns levels measured in control compared to cystic kidneys normalised to Hprt. n=5, two technical replicates of at least 2 independent experiments. Microarrays from Pkd1^{V/V} P10 kidneys and human-derived microarrays of PKD patients samples showing upregulation of ASNS. (10B) Quantitative RT-PCR of the genes coding for glutaminase (GLS) or glutamine dehydrogenase (GLUD1), two key enzymes involved in glutamine usage (top scheme) normalised to Hprt shows that they are not increased in Pkd1^{-/-} MEFs, cystic kidneys and human-derived microarray of PKD patients compared to the relative controls. (10C) mRNA levels of Asns in Pkd1^{-/-} compared to Pkd1^{-/-} (mock) cells. Graphs represents. Mean+/−SEM were indicated, n.s., not significant (p≥0.05), *p<0.05; **p<0.001; ***p<0.0001; ****p<0.00001. t-test used for (10A, 10B and 10C). (10A and 10B) four independent biological replicates for Pkd1^{V/V} animal model, eight (normal and minimal cyst) and 13 independent biological replicates for large, medium, small cyst for human PKD1 microarrays (10A and 10B). Graphs in (10B) corresponding to the MEFs are representative of means of data of two independent experiments made on three experimental replicates and for the mice they are representative of five independent biological replicates (10B) and three independent biological replicates (10C).

FIGS. 11A-E. (11A) KEGG-Pathways Based Enrichment Analysis applied to the list of metabolites deriving from in silico simulation of increased glycolysis ranked according to the DFA algorithm. Only human pathways for which at least five metabolites are present in our mathematical model have been considered. Picture shows that 31 pathways result in an enrichment scores having FDR≤0.01, where FDR has been computed by applying Benjamini-Hochberg procedure. (11B) Quantitative RT-PCR of genes encoding Cpt1a show decreased in Pkd1^{-/-}, MEFs and cystic kidneys compared to controls. (11C) Clustergram performed non-supervised hierarchical clustering of the entire dataset displays a heat map with co-regulated genes across groups (cystic Vs. controls) for Glycolysis, Gluconeogenesis, PPP, TCA, FAS and FAO. The p values are calculated based on a Student's t-test of the replicate 2\hat{ }(−Delta C_{T}) values for each gene in the control group and treatment groups, and p values less than 0.05 are indicated in red. (11D) Principal component analysis applied to the genes in microarrays data from Pkd1^{V/V} animal model at P10 shows good separation between mutant versus control. (11E) Hierarchical clustering analysis applied to the genes in microarrays data from Pkd1^{V/V} animal model at P10 shows separation between mutant versus control. FQ, corresponds to user identification sample code.

FIGS. 12A-B. (12A) In case of Asparagine depletion, the Amino Acid Response pathway (AAR) is activated, Unfolded Protein Response is activated in case of endoplasmic reticulum stress (ER Stress). The activation of one of these pathways increases the activity of an eIF2 kinase. Phosphorylation of eIF2 increases the translation of the transcription factor ATF4. ATF4 binds then to an enhancer element within the promoter of the ASNS gene inducing its expression. (12B) Human-derived microarrays of PKD patients samples show upregulation of ATF4.

FIGS. 13A-B. LNAs knockdown for Asns. (13A) LNAs targeting Asns were transfected in Pkd1^{-/-} MEFs respective to mock cells, achieving almost complete knockdown in #1, #2, #3 and #5. (13B) Negative and positive controls Metastasis Associated Lung Adenocarcinoma Transcript 1 (Malat1) was tested in Pkd1-MEFs respective to mock cells. mRNA Asns and controls were normalized to Hprt. Mean±SEM were indicated, n.s. not significant (p≥0.05), *P<0.05; ANOVA followed by Bonferroni.

FIGS. 14A-C. LNAs knockdown for Asns. (14A) Representative images showing cellular morphology in LNAs targeting Asns were transfected in Pkd1^{-/-} MEFs #1 and 2 respective to mock cells. (14B) Cell count, expressed as percentage respective to mock controls. (14C) mRNA expression for Asns Pkd1^{-/-} MEFs respective to mock cells. mRNA Asns and controls were normalized to Hprt. Mean±SEM were indicated, n.s. not significant (p≥0.05), *P<0.05; **P<0.01; ***P<0.001; ****P<0.0001. ANOVA followed by Bonferroni.

FIGS. 15A-B. LNAs knockdown for Asns. (15A) Representative images showing cellular morphology in LNAs targeting Asns were transfected in mCCD mutant for Pkd1 (Pkd1^{-/-}) and respective mCCD controls (Pkd1^{+/+}). Images showing mock and LNAs for #1, #2 and #3 and mock in Pkd1^{-/-} and LNAS #1 and mock for Pkd1^{+/+}). (15B) mRNA expression for Asns in mCCD mutant for Pkd1, respective to mock cells. mRNA Asns and controls were normalized to Hprt. Mean±SEM were indicated, n.s. not significant (p≥0.05), *P<0.05; **P<0.01; ***P<0.001; ****P<0.0001. ANOVA followed by Bonferroni.

FIGS. 16A-D. (16A) Schematic overview of the experimental plan, Pkd1^{ΔC/floxTmCre} mice were injected with a single dose of Tamoxifen (Tam, 10 mg/40 g) at P24. Treatments with either ASO #1 and/or ASOcontrols was performed once per week at 50 mg/kg for a total of 7 weeks and sacrificed at P94. (16B) Representative images of cystic kidney treated with ASOcontrols and ASO #1 (litter 1). (16C) The percentage of kidneys to total body weight in cystic mice treated mice respect to ASOcontrols. (16D) BUN was evaluated at the time of sacrifice, showing a reduction in the cystic treated animals (for Litter D, one blood sample was not available) Mean±SEM were indicated, n.s. not significant (p≥0.05), *P<0.05; **P<0.01; ***P<0.001; ****P<0.0001. Paired t-test was used for the cystic animals treated with #ASOcontrols Vs. #ASO1.

FIG. 17. ASOs knockdown for Asns. mRNA levels for Asns and controls, showing a reduction in Asns levels. mRNA relative expression and normalized to Hprt. Mean±SEM were indicated, n.s. not significant (p≥0.05), *P<0.05. Paired t-test was used for the cystic animals treated with #ASOcontrols Vs. #ASO1.

FIG. 18. Liver toxicity assessed by ALT. Alanine aminotransferases (ALT) was assessed for the treated animals, showing no significance in the cystic kidneys group. Mean±SEM were indicated, n.s. not significant (p≥0.05), *P<0.05. Paired t-test was used for the cystic animals treated with #ASOcontrols Vs. #ASO1.

FIG. 19. Four litters were collected each containing intra-litters treated animals and controls animals. Litter 1,2, and 3, animals were treated for a weekly injection of a total of 7 per week. Litter 4, animals were treated for a weekly injection of a total of 6 per week. As described below:
Litter 1:4 animals, males sacrificed at P94
Litter 2:4 animals, females sacrificed at P94
Litter 3:4 animals, females sacrificed at P94
Litter 4:4 animals, males sacrificed at P88

FIG. 20. Growth assay on cells after 72 h of treatment with different concentrations of cpd 17.

FIG. 21. Time course growth assay on cells treated for 72 h with different concentrations of cpd 17.

FIGS. 22A-B. Growth assay (22A) and Cytotoxic evaluation (22B) in MEFs at 24, 48, 72 h of treatment with 30 μM cpd 17.

FIG. 23. Mean (+/−SD) plasma concentration (μM) vs time profile of cpd 17. Cpd 17 was administered to female C57BL/6 mice via intravenous (I.V.), oral (P.O.) or intraperitoneal (I.P.) route. The dotted line shows PPB-corrected IC50 (˜2 μM).

FIG. 24. Schematic representation of target engagement study design.

FIGS. 25A-D. Growth curves of WT (25A) and KO (25B) mice enrolled in the study. (25C) Concentrations of cpd17 in the kidneys collected from WT and KO mice treated with 10 mg/kg or 30 mg/kg of cpd17 for 2 weeks. (25D) Asparagine quantification in cystic kidneys treated with cpd 17 or vehicle. The numbers indicate micro molar concentrations.

FIG. 26 is a table, identified as Table I, which lists genes belonging to glycolysis, pentose phosphate pathway, TCA cycle/OXPHOS, fatty acid synthesis and fatty acid oxidation.

### DETAILED DESCRIPTION OF THE INVENTION

### Materials and Methods

Generation of Pkd1^{flox/-}:Ksp-Cre mice

Pkd1^{flox/-}:Ksp-Cre is a mouse model for ADPKD that develops massive enlarged kidney cysts within few days after birth and was generated by crossing Pkd1^{flox/flox} and Pkd1^{+/-}:Ksp-Cre mice^{32,33}. The age of the pups was accurately assessed by daily control of birth combined with the follow up of the variation of coat colors as described by Jackson

Laboratories (https colon forward slash forward slash www dot jax dot org). All mice used in these experiments were in a pure C57/BL6N genetic background (i.e. >10 backcrosses) and were maintained in specific pathogen free colonies (SPF) handled by a service company provided at the San Raffaele Scientific Institute (Charles River). Mice received a sterilized (vacuum packed and irradiated) chow diet [25/18 CR, 5% w/w crude fat (predominantly from soya products), soya oil 0.5% (14% kcal from fats, energy density of 2.64 kcal/g)]. All mice had ad libitum access to water and food. All experiments involving animals were performed under a protocol approved by an institutional ethical committee and, subsequently, by the Italian ministry of health (IACUC number: 736).

### Untargeted Metabolomic Analysis of Kidneys and MEFs

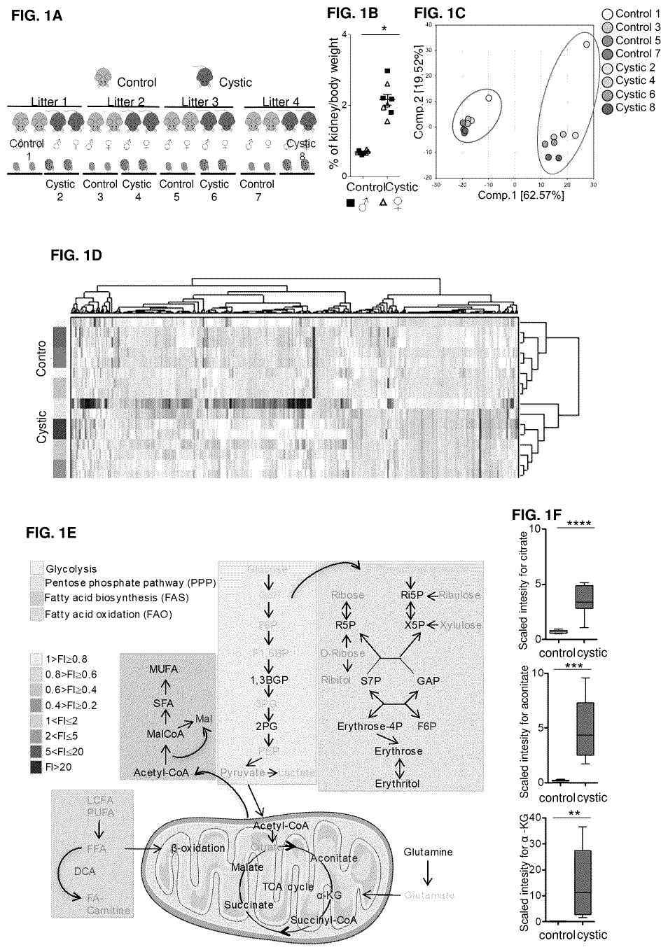

The untargeted metabolomics in kidneys (FIG. 1) and MEFs (FIG. 8) were carried out at Metabolon®). Samples were subjected to methanol extraction, split into aliquots for analysis by ultrahigh performance liquid chromatography/mass spectrometry (UPLC/MS). Thermo Scientific Q-Exactive high resolution/accurate mass spectrometer interfaced with a heated electrospray ionization (HESI-II) source and Orbitrap mass analyzer operated at 35,000 mass resolution. The sample extract was dried then reconstituted in solvents compatible to each of the four methods. Each reconstitution solvent contained a series of standards at fixed concentrations to ensure injection and chromatographic consistency. One aliquot was analyzed using acidic positive ion conditions, chromatographically optimized for more hydrophilic compounds. In this method, the extract was gradient eluted from a C18 column (Waters UPLC BEH C18-2.1×100 mm, 1.7 μm) using water and methanol, containing 0.05% perfluoropentanoic acid (PFPA) and 0.1% formic acid (FA). Another aliquot was analyzed using acidic positive ion conditions, however it was chromatographically optimized for more hydrophobic compounds. In this method, the extract was gradient eluted from the same afore mentioned C18 column using methanol, acetonitrile, water, 0.05% PFPA and 0.01% FA and was operated at an overall higher organic content. A third aliquot was analyzed using basic negative ion optimized conditions using a separate dedicated C18 column. The basic extracts were gradient eluted from the column using methanol and water, however with 6.5 mM Ammonium Bicarbonate at pH 8. The fourth aliquot was analyzed via negative ionization following elution from a HILIC column (Waters UPLC BEH Amide 2.1×150 mm, 1.7 μm) using a gradient consisting of water and acetonitrile with 10 mM Ammonium Formate, pH 10.8. The MS analysis alternated between MS and data-dependent MSⁿ scans using dynamic exclusion. The scan range varied slighted between methods but covered 70-1000 m/z. Raw data files are archived and extracted as described below. Metabolite concentrations were determined by automated ion detection, manual visual curation and were analysed in-line using software developed by Metabolon®³⁴.

Bioinformatics: The informatics system consisted of four major components, the Laboratory Information Management System (LIMS), the data extraction and peak-identification software, data processing tools for QC and compound identification, and a collection of information interpretation and visualization tools for use by data analysts. The hardware and software foundations for these informatics components were the LAN backbone, and a database server running Oracle 10.2.0.1 Enterprise Edition. LIMS: The purpose of the Metabolon LIMS system was to enable fully auditable laboratory automation through a secure, easy to use, and highly specialized system. The scope of the Metabolon LIMS system encompasses sample accessioning, sample preparation and instrumental analysis and reporting and advanced data analysis. All of the subsequent software systems are grounded in the LIMS data structures. It has been modified to leverage and interface with the in-house information extraction and data visualization systems, as well as third party instrumentation and data analysis software. Data Extraction and Compound Identification: Raw data was extracted, peak-identified and QC processed using Metabolon's hardware and software. These systems are built on a web-service platform utilizing Microsoft's .NET technologies, which run on high-performance application servers and fiber-channel storage arrays in clusters to provide active failover and load-balancing. Compounds were identified by comparison to library entries of purified standards or recurrent unknown entities. Metabolon maintains a library based on authenticated standards that contains the retention time/index (RI), mass to charge ratio (m/z), and chromatographic data (including MS/MS spectral data) on all molecules present in the library. Furthermore, biochemical identifications are based on three criteria: retention index within a narrow RI window of the proposed identification, accurate mass match to the library+/−10 ppm, and the MS/MS forward and reverse scores between the experimental data and authentic standards. The MS/MS scores are based on a comparison of the ions present in the experimental spectrum to the ions present in the library spectrum. While there may be similarities between these molecules based on one of these factors, the use of all three data points can be utilized to distinguish and differentiate biochemicals. More than 3300 commercially available purified standard compounds have been acquired and registered into LIMS for analysis on all platforms for determination of their analytical characteristics. Additional mass spectral entries have been created for structurally unnamed biochemicals, which have been identified by virtue of their recurrent nature (both chromatographic and mass spectral). These compounds have the potential to be identified by future acquisition of a matching purified standard or by classical structural analysis.

Curation: A variety of curation procedures were carried out to ensure that a high quality data set was made available for statistical analysis and data interpretation. The QC and curation processes were designed to ensure accurate and consistent identification of true chemical entities, and to remove those representing system artefacts, mis-assignments, and background noise. Metabolon data analysts use proprietary visualization and interpretation software to confirm the consistency of peak identification among the various samples. Library matches for each compound were checked for each sample and corrected if necessary.

### Lipid Extraction for Untargeted Mass Spectrometry Profiling

Mass spectrometry-based lipid analysis was performed at Lipotype GmbH (Dresden, Germany) and lipids were extracted using a two-step chloroform/methanol procedure. Samples were analyzed by direct infusion on a QExactive mass spectrometer (Thermo Scientific) equipped with a TriVersa NanoMate ion source (Advion Biosciences). Samples were analyzed in both positive and negative ion modes with a resolution of Rm/z=200=280000 for MS and Rm/z=200=17500 for MSMS experiments, in a single acquisition. MSMS was triggered by an inclusion list encompassing corresponding MS mass ranges scanned in 1 Da increments as previously described³⁵. Briefly, samples were spiked with internal lipid standard mixture containing: cardiolipin 16:1/15:0/15:0/15:0 (CL), ceramide 18:1;2/17:0) (Cer), diacylglycerol 17:0/17:0 (DAG), hexosylceramide 18:1;2/12:0) (HexCer), lyso-phosphatidate 17:0 (LPA), lyso-phosphatidylcholine 12:0) (LPC), lysophosphatidylethanolamine 17:1 (LPE), lyso-phosphatidylglycerol 17:1 (LPG), lyso-phosphatidylinositol 17:1 (LPI), lyso-phosphatidylserine 17:1 (LPS), phosphatidate 17:0/17:0 (PA), phosphatidylcholine 17:0/17:0 (PC), phosphatidylethanolamine 17:0/17:0 (PE), phosphatidylglycerol 17:0/17:0) (PG), phosphatidylinositol 16:0/16:0 (PI), phosphatidylserine 17:0/17:0 (PS), cholesterol ester 20:0 (CE), sphingomyelin 18:1;2/12:0;0) (SM), triacylglycerol 17:0/17:0/17:0 (TAG) and cholesterol D6 (Chol). After extraction, the organic phase was transferred to an infusion plate and dried in a speed vacuum concentrator. First step dry extract was re-suspended in 7.5 mM ammonium acetate in chloroform/methanol/propanol (1:2:4, V:V:V) and second step dry extract in 33% ethanol solution of methylamine in chloroform/methanol (0.003:5:1; V:V:V). All liquid handling steps were performed using Hamilton Robotics STARlet robotic platform with the Anti Droplet Control feature for organic solvents pipetting.

### Targeted Metabolomics and Stable Isotope Tracer Analysis

Immortalized Pkd1^{+/+} and Pkd1^{-/-} MEFs³⁶ and SiAsns Pkd1^{-/-} were plated at 150,000 cells/well onto a 6-well plate (n=6) and cultured in standard conditions. 24 hrs before sampling for ¹³C-labelling experiments, MEFs were washed twice with PBS and supplemented with media (DMEM (Gibco) supplemented with 10% dialysed foetal bovine serum (Gibco), 1% Penicillin Streptomycin (Pen/Strep, Gibco), Sodium Bicarbonate (3.7 g/l; Sigma Aldrich) containing either uniformly labelled ¹³C₆-glucose (25 mM) (Cortecnet), ¹³C₅-¹⁵N₂-glutamine (4 mM) (Cortecnet), ¹⁵N₂-Glutamine (Cambridge Isotope Laboratories), or ¹³C₅-Glutamine (Cambridge Isotope Laboratories). Metabolites were extracted from cell pellets (intracellular) with 1 mL of extraction solution (methanol for highly pure liquid chromatography (Sigma Aldrich): acetonitrile gradient grade for liquid chromatography (Merck):ultrapure water (Sigma Aldrich), 50:30:20 with 100 ng·ml⁻¹ of HEPES (Sigma Aldrich) per million cells. The cell culture medium (extracellular) extracts were prepared by adding 750 μL of extraction solution to 50 μL of centrifuged cell culture medium. Samples were incubated at 4° C. for 15 min at 700 r.p.m., before centrifugation at 13,000 r.p.m. The supernatant was transferred into autosampler vials and stored at −80° C. prior to analysis by liquid chromatography coupled to mass spectrometry (LC-MS). LC-MS analysis was performed using a Q Exactive Orbitrap mass spectrometer coupled to a Dionex U3000 UHPLC system (Thermo Fisher Scientific). The liquid chromatography system was fitted with a Sequant ZIC-PHILIC column (150 mm×2.1 mm) and guard column (20) mm×2.1 mm) from Merck Millipore and temperature maintained at 45° C. The mobile phase was composed of 20 mM ammonium carbonate and 1% ammonium hydroxide in water, and acetonitrile. The flow rate was set at 200 μL·min⁻¹ with the gradient described previously³⁷. To expand on the range of metabolites covered in the analysis, the sample extracts were then run on a ZIC-HILIC column (150 mm×4.6 mm) fitted with a guard column (20 mm×2.1 mm) (both Merck Millipore). The aqueous mobile phase solvent used was 0.1% formic acid in water, and the organic mobile phase was 0.1% formic acid in acetonitrile. The flow rate was set at 300 μl·min⁻¹ and the column oven set to 30° C., as described previously³⁷. The mass spectrometer was operated in full MS mode with polarity switching, and samples were randomised in order to avoid bias due to machine drift and processed blindly. The acquired spectra were analysed using XCalibur Qual Browser and XCalibur Quan Browser software (Thermo Fisher Scientific). Mass isotopologue distribution of metabolites was determined by integration of the corresponding peaks, and correction for natural abundance was performed using the Polly™ IsoCorrect tool from the cloud-based platform Elucidata (https colon forward slash forward slash polly dot elucidate dot io).

Consumption and release of metabolites was assessed by subtracting each metabolite total pool in the fresh medium (incubated in the absence of cells) from the pool found in the spent medium samples. The resulting value was then adjusted to the amount of protein generated during the incubation of the cells with the labelled substrate. For this purpose, cells were seeded in parallel plates and protein content was determined by the Bradford method at 0 and 24 hrs post medium change. Percentage of intracellular pool from each isotopologue was calculated respective of the control (for each metabolite).

### Glycolysis and Mitochondrial Respiration Assays

Cells were plated at a density of 20,000 or 30,000 cells per well in a 96-wells Seahorse cell culture microplates and incubated in a 5% CO₂ incubator at 37° C. overnight. The following day, 1 hr before the test, culture media was replaced with pH-adjusted (pH=7.4±0.1) bicarbonate-free DMEM (Agilent) with 10 mM glucose (Sigma Aldrich), 1 mM sodium pyruvate (Gibco), and 2 mM L-glutamine (Gibco) for Mito Flex Test (Agilent) and Mito Stress Test or with 2 mM L-Glutamine only for Glycolysis Stress Test (Agilent). The plate was then incubated at 37° C. for 1 hr in a non-CO₂ incubator. For the Mito Fuel Flex test and Mito Stress Test, Oxygen Consumption Rates (OCR) were measured using the Seahorse XF Mito Fuel Flex Test Kit XF and Mito Stress Test Kit (Agilent). Extracellular Acidification Rate (ECAR) was measured using XF Glycolysis Stress Test Kit (Agilent) on an XFe96 Analyzer (Agilent) following the manufacturer's instructions. Cell numbers were normalized using CyQuant (Thermo Fisher).

### RNA Extraction and Microarray

Kidneys were removed from wild type (n=4) and Pkd1^{V/V} (n=4) mice at P10 and homogenized in PBS buffer using mini handheld homogenizer. The homogenate was centrifuged and supernatant was discarded. Total RNAs were extracted from the pellet using an RNeasy mini kit (Qiagen) and the quality of total RNA samples was verified by a 260/280 ratio in NanoDrop and agarose gel electrophoresis. Further sample processing for microarray analysis was performed by the Genomics Core of Cleveland Clinic's Lerner Research Institute, following the facility's protocols, and hybridized to Illumina's MouseRef-8 v2.0 BeadChip expression arrays.

### Real-Time PCR Analysis

RNA was isolated from plated cells or snap-frozen kidneys using the RNAspin Mini kit (GE Healthcare). Total RNA was isolated using the RNA-Isol Lysis reagent according to the manufacturer's instructions. For reverse transcription of RNA, Oligo (dT) 15 primers (Promega) and ImProm-II Reverse Transcriptase (Promega) were used. Quantitative real-time PCR analysis was performed on duplicate using SYBR Green I master mix (Roche) on LightCycler 480 Instrument (Roche). The following primers were used for quantitative RT-PCR: Epo forward 5′-CATCTGCGACAGTCGAGTTCTG-3′ SEQ ID NO:1 reverse5′-CACAACCCATCGTGACATTTTC-3′ SEQ ID NO:2; Vefg forward 5′-CTGTGCAGGCTGCTGTAACG-3′ SEQ ID NO:3 reverse 5′GTTCCCGAAACCCTGAGGAG-3′ SEQ ID NO:4; Fasn forward 5′-TTGCTGGCACTACAGAATGC-3′ SEQ ID NO:5 reverse 5′-AACAGCCTCAGAGCGACAAT-3′ SEQ ID NO:6; Cpt1a forward 5′-AGTGGCCTCACAGACTCCAG-3′ SEQ ID NO:7 reverse 5′-GCCCATGTTGTACAGCTTCC-3′ SEQ ID NO:8; Glud1 forward 5′-CCCAACTTCTTCAAGATGGTGG-3′ SEQ ID NO: 9 reverse 5′ AGAGGCTCAACACATGGTTGC-3′ SEQ ID NO:10, Gls forward 5′-TTCGCCCTCGGAGATCCTAC-3′ SEQ ID NO: 11 reverse 5′ CCAAGCTAGGTAACAGACCCT-3′ SEQ ID NO:12; Asns forward 5′-GGTTTTCTCGATGCCTCCTT-3′ SEQ ID NO:13 reverse 5′-TGTGGCTCTGTTACAATGGTG-3′ SEQ ID NO:14; Hprt forward 5′-TTATGTCCCCCGTTGACTGA-3′ SEQ ID NO:15 reverse 5′-reverse ACATTGTGGCCCTCTGTGTG-3′ SEQ ID NO:16.

### Transient Knockdown of Fasn and Asns

For transient knockdown of siFasn and SiAsns 20 nM (Ambion) with the following sequences:

FASN 5′-GGGAUCAUAAAGAUAACUUtt-3′ SEQ ID NO:17 and 5′-AAGUUAUCUUUAUGAUCCCtc SEQ ID NO:18 ASNS 5′-GGCCCUUGUUUAAAGCCAUtt-3′ SEQ ID NO:19 and 5′-AUGGUUUAAACAAGGGCCtg-3′ SEQ ID NO:20 along with control scrambled siRNA: siCONTROL or mock (only transfection reagent) (#AM4613, Dharmacon) were used following the manufacturer's instructions and transfection control. For siRNA transfection cells were seeded into a 6 well plate, 100,000/well in 10% FBS (Gibco) in DMEM (Gibco) without antibiotics. The transfections were performed two times over 2 days at a final concentration of 30 nM using Lipofectamine 3000® following the manufacturer's protocol. Total RNA was prepared from the cells 72 hrs after the first transfection and qRT-PCR was performed.

### Apoptosis Cell Assay

Cell death detection kit (TUNEL, Promega) was performed according to the manufacturer instructions after transient knockdown of siFasn or after 48 hrs of starvation experiments (glutamine, glucose starvation). The protocols and quantifications were previously optimized¹¹.

### Growth Curve of MEFs in Glucose and Glutamine Starvation

Immortalized Pkd1^{+/+} and Pkd1^{-/-} MEFs were plated at a density of 150,000 cells/well in DMEM (Gibco) supplemented with 0.5% FBS (Gibco) and 1% Pen/Strep (Gibco). After 16 hrs, medium was changed to control medium (DMEM, Gibco), supplemented with 10% FBS (Gibco), 1% Pen/Strep (Gibco), Sodium Pyruvate (1 mM; Gibco), Sodium Bicarbonate (44 mM; Sigma Aldrich), 25 mM Glucose (Sigma Aldrich) and 4 mM Glutamine (Gibco); Glucose starvation medium (DMEM supplemented with 1 mM Sodium Pyruvate, 44 mM Sodium Bicarbonate, 10% FBS, 1% Pen/Strep and 4 mM Glutamine); Glutamine starvation medium (DMEM supplemented with 10% FBS, 1% Pen/Strep, 1 mM Sodium Pyruvate, 44 mM Sodium Bicarbonate and 25 mM Glucose) or Glucose and Glutamine starvation medium (DMEM supplemented with 10% FBS and 1% Pen/Strep, 1 mM Sodium Pyruvate, 44 mM Sodium Bicarbonate). After 24 hrs and 48 hrs, cells were trypsinized and counted with an automated cell counter (Countess cell counter, Invitrogen). Pictures from each sample at both time points were taken with a white field microscope, using a 10× objective.

### Analysis Using the KEGG-Pathways Based Enrichment Analysis

Analysis of the untargeted metabolomics studies was performed by implementing a KEGG-Pathways Based Enrichment Analysis (PBEA) system based on a similar concept than the Gene Set Enrichment Analysis (GSEA)³⁸ which tests whether compounds involved in predefined pathways occur towards the top or bottom of the ranked query list. The method identifies altered pathways of the Kyoto Encyclopaedia of Genes and Genomes (KEGG)³⁹ using metabolite data. An enrichment score(ES) and a statistical significance (p-value) have been computed for each pathway having at least one metabolite captured in the list of metabolites ranked, in descending order, according to a specific metric. Particularly, a fold-change was used to rank the metabolites deriving from non-targeted global metabolomics, while the DFA algorithm (see below) was used to rank the compounds resulting from the in silico simulations. In order to avoid ambiguous identification and to obtain reliable results only metabolites having a KEGG-identifier have been considered. The list of ranked metabolites named as L, and S for the set of compounds of a particular pathway. Analysis is performed to test whether the elements of S are randomly distributed through L or primarily found at the top or bottom of the list. ES reflects the degree to which S is over-represented at the extremes of L, and it is computed by walking down L, increasing a running-sum statistic when a metabolite in S is found and decreasing it when a compound not in S is encountered. The obtained score corresponds to a weighted Kolmogorov-Smirnov-like statistic³⁹ and represents the maximum deviation from zero associated with a random walk. The p-value associated with an ES has been computed as the fraction of 1000 random permutations of the elements of L that are at least as extreme as the original ES, that has been derived from non-permuted elements. A Matlab® implementation of the method is available upon request.

### Differential Abundance Score

For a particular pathway P, the Differential Abundance (DAₚ) score is defined as:

$$D {A}_{P} = \frac{{\sum }_{r ∈ P} {u}_{r}}{{\sum }_{r ∈ P} \vert {u}_{r} \vert}$$

where uᵣ is the delta of in silico flux of reaction r computed by DFA. This score captures the tendency for a pathway to be up-regulated or down-regulated and varies from −1 to 1. A score of −1 indicates that all the metabolic fluxes associated with reactions in pathway P decreased respect to WT, while a score of 1 indicates that all fluxes increased when comparing with the WT simulation.

### In Silico Modelling and Simulation of Increased Glycolysis

For the in silico studies the Genome-Scale Metabolic Model described in Pagliarini et al. was applied⁴⁰. The model comprises 785 metabolites and 2589 enzymatic and transport reactions in eight compartments. In order to have more physiological results, the following additional constrains have been imposed i) citrate cannot move from cytosol to mitochondria, ii) pyruvate cannot move from mitochondria to cytosol, and iii) cytosolic enzyme LDH can only convert pyruvate to lactate and not vice-versa. Then, a recent algorithm named Differential Flux-balance Analysis (DFA) was applied⁴⁰ to simulate either the wild-type condition (WT) or an increase of glucose entering the cytosol (GLY) and DFA was applied to the two conditions. DFA is based on solving a linear optimization problem across 442 metabolic objectives for both the WT model and the perturbed model. For the current study, the liver-specific metabolic functions were removed. The average flux carried by each reaction across the different metabolic objectives for each of the two models is computed. For each reaction, the difference of the average flux in the WT model minus its value in the modified model are then considered. These differential fluxes are then used to rank the reactions from the ones that change the most in the modified model to the ones that change the least or do not change at all. Metabolites are then ranked according to the sum of the absolute values of the differential fluxes. In order to simulate an increase of the glucose uptake the results of the ¹³C-glucose labelled experiments showing an increase of 1.6 fold-change in the uptake of glucose has been used. Therefore, the fluxes through the reactions Glucose(s)+Na+(s)->Glucose (c)+Na+(c) (TCDB: 2.A.21.3.6) and Glucose(s)<=>Glucose (c) (TCDB: 2.A.1.1.29) has been forced, to be equal or greater than a specific threshold set to the their average value obtained in the WT simulations increased of 60%.

### Analysis of Murine and Human Microarray Data

In order to infer differentially expressed genes in microarrays data from Pkd1^{V/V} animal model at P10, a Matlab®) implementation of Significance Analysis of Microarrays (SAM) algorithm with delta=1.0 was applied.

For human microarrays, data from Song et al.⁴¹ have been considered. First, affymetrix probe sets have been collapsed to one gene level by using the maximum expression value of the probe set in each gene. Then, SAM algorithm has been applied, with delta=2.4, to identify differentially expressed genes between transcripts in renal cysts of different sizes, and minimal cystic tissue plus normal renal cortical samples. The Matlab® implementation of SAM can be found in (https colon forward slash forward slash it.mathworks.com forward slash matlabcentral/fileexchange/42346-significance-analysis-of-microarrays--sam--using-matlab). In both microarrays analysis, after the computation of all the differentially expressed genes, those belonging to glycolysis, pentose phosphate pathway, TCA cycle/OXPHOS, fatty acid synthesis and fatty acid oxidation were identified in Table I, see FIG. 26.

Table I. List of genes belonging to glycolysis, pentose phosphate pathway, TCA cycle/OXPHOS, fatty acid synthesis and fatty acid oxidation.
Glycolysis: HK1, HK2, HK3, HKDC1, GCK, GCKR, ADPGK, PFKFB1, PFKFB2, PFKFB3, PFKFB4, PFKL, PFKM, PFKP, PKM2, PKLR, PGM1, PGM2, PGM3, GALM, GP1, ALDOA, ALDOB, ALDOC, TPI1, GAPDH, GAPDHS, PGK1, PGK2, BPGM, PGAM1, PGAM2, PGAM5, MINPP1, ENO1, ENO2, ENO3, LDHA, LDHAL6A, LDHAL6B, LDHB, LDHC, LDHD
Pentose Phosphate Pathway: GPI, G6PD, H6PD, PGLS, PGD, RPE, RPIA, RBKS, PGM2, PGM1, PRPS1, PRPSIL1, PRPS2, PRPSAP1, PRPSAP2, TKT, TKTL1, TKTL2, FBP1, TKT, TKTL1, TKTL2, RBKS, DERA
TCA/OxPhos: PC, CS, ACLY, ACO1, ACO2, IDH1, IDH2, IDH3A, IDH3B, IDH3G, OGDH, DLST, SUCLA2, SUCLG1, SUCLG2, SDHA, FH, MDH1, MDH1B, MDH2, FAS, FADS1, FADS2, FADS3, ACOX1, ACOX2, ACOX3, ELOVL1, ELOVL2, ELOVL3, ELOVL4, ELOVL5, ELOVL6, ELOVL7, HSD17B12, SCD, SCD5, CD36, SLC27A1, SLC27A2, SLC27A3, SLC27A4, SLC27A5, FABP1, FABP2, FABP3, FABP4, FABP5, FABP6, FABP7, APOA1, ME1, LPL, SREBF1, FASN, FAS, FAO, CPT1A, CPT2, ACADVL, EHHADH, ACAA2, ACAA1, ACADM, EHHADH, ACADVL, ACADL, ACADM, ECHS1, HADH, HADHA.

### Statistical Analysis

For statistical analysis the Prism 5, GraphPad Software and Matlab® were used using statistical analysis tool. T-test was used for all analysis of two groups. ANOVA statistical analysis followed by Bonferroni's multiple comparison test was performed in all analysis where more than two groups were present. The P-values for each condition are indicated in the legends.

Design of antisense LNA GapmeRs

Design of antisense locked nucleic acids (LNA) GapmeRs were designed via Exiqon's/Qiagen Antisense LNA™ Gapme® design algorithm by uploading Asns [Mus musculus] FASTA transcript sequence from NCBI resource.
NC_000072.6:c7693182-7675165 Mus musculus strain C57BL/6J chromosome 6, GRCm38.p4 C57BL/6J
https colon forward slash forward slash www dot qiagen dot com forward slash it/shop/genes-and-pathways/custom-products/custom-functional-analysis/antisense-lna-gapmers-designer/
10 Antisense LNA GapmeRs were designed, the 5 top design LNA GapmeRs (#1, #2, #3, #4, #5) were selected and ordered.

In bold are highlighted the 5 sequences corresponding to the 5 top design (#1, #2, #3, #4, #5) and targeting of the primary transcript as shown in the FASTA sequence. Controls were selected negative LG00000002 and positive control Malat1 (mouse) LG00000008.

LG00220663 #1
SEQ ID NO: 21
rev com. CTAACTAACCAATGCG
LG00220664-DDA #2
SEQ ID NO: 22
rev com TAGTCAAATGCCAATC
LG00220665-DDA #3
SEQ ID NO: 23
rev com GTTATTGTAACGACGA
LG00220666-DDA #4
SEQ ID NO: 24
rev com TTAATTCTTGACACGG
LG00220667-DDA #5
SEQ ID NO: 25
rev com TTCTCCTAGCTAACGT
Asns [Mus musculus] FASTA transcript sequence from NCBI resource (in bold
corresponding sequences of targets)
SEQ ID NO: 26
AAGCGGCCTCCAACCGGTCTTGTCACTGCGCTGCCTCTGCTCCACCTTCTCTGGCCCTGGCCGCTAGTGCTCAGGT
AAGCGGGAGCCGGGTGCTCAGCGCGGAGCGCTCTGCTCGCCTGGGAGGCCGGAGGATGTGCTGTCCGCGCGGGCAC
ATGTCCCGGGAGTTCGGATGCGGAGCGCTCGGGTCCGGCGCCAGACCGATGCTGCGGTGCAGCGCGAGGCAGGGCG
GGGCGGGCGCGGGGCGATTGTTGCGCACCTAGGACGGAGCTGCCAGGAAAGCTTTCATGTGAGCGAAGTGCAGCGC
GGAGGCAGGGCCGCTCCAGACAGAAGGCGAAGGTGGCCTTCATTTAGACACCATAAATATTTTTTTTTACTGTATG
ACGTCTAGATCTGGGAAATTTCAGCCATCCGAAGGTTTTGCCTGGGACTAGGTGGACGCTTGCACAAGACCAGCTG
TAATTGGGAGCCCCAGGCTAGTGTAGGGGTCTCGCTCTCCAGCAGGACAGAGGCTCTACCAAGCTCTAGAGCGAGT
GTCTCTGGGTCTGTCTGGATCCGGATTTCTGAGCACTCACTCACCTCGCTGCTCAGGCCCTTCCTCCACTCCAGTT
CCATACTCGCAGCCACGCCGGGAAAAATCTGTTTAAGGAGACAGGCAACCAAGTACGGGAAAAGGCAAAGGAATTG
GCGTGCTTTTTTTTTTTTTTTTTTTTTTTTCTTAATTATGGATAGCTATTTCACAGGTCTAGTTTCAGATAAGATA
ACTTTTAAAATACACGGGCGAGTTTGATTTTTCGTTGCCTGTGGCCTACGGGTTTTAACAGCTAACTCTTCGTTGA
TTGATGTCAGTACTATAATATTCATTTGAAGTAAGACTTGCACAAGGCCACGGGTTGATTATTGAGGTCTTAAGGT
GATCCAGTGGAGTTTAGAACTATCAGTTCAGTGTACGAAAGCGGCGCTGTCCAGCGGGGAGCCTGTGCTCTTTCAT
AGACTGACCCCTAGAAATGCTGGCTAGTCTGAACTGAGATGTGGTAAGAGTGTAATGTGTCGATTTCGATGATTAT
GAAAACCGAACGCTTGATGTTTTGATGTTTGATTGCAGCCTCTAACCACTAAAGTATCCCTCCAGCCCGGTTTTAA
TAATTTAGGAAAAGTAGTGTAAATATAATTAATTTTGATTATATTGTATTGGTTGTCGTATTTAGGGTGTGTCATG
TTAAGGTATGTTTAAAATTAGTTTTAATGTTTTCTCTTGTCTTCTTAATGAGCTTTATTAGTATATTAAAATTTCA
CATATGGCTCACTGGCCAGTACTGCTGCAGAAGTTAACCAGGAAAGGAGGAAATTTACAGACAACAGTGAGGAAAA
TATGGTACAAGAGGGCCTCTGAGTCCGATTACAAAGGAGTTTGAATTTCCTTACTCCTGCTGAAGGAAAGAATTTT
AAACTTTTCTTTACACTTCAATGTAGTGCCTGGCATATGTTTTGCATACCAGACAGTTCATTAAAGAGCCTTAAGA
CCTAGGTAAATGGTTCTCGGTGATCATAATTGATTTTCAGAGCGCTTTGTTAGCCCACTTCCCTTTCCCTTTTATT
AGACATAGATTCTTTTCTCATATAATACTATCTGAACAGTTTCCCACCTCTCTACTTATAGTGTCTCCCCACCTCT
CCCCTCTGAATTCACCCCTTTTCTGCCTCTCATTAGAAAAGGATAGGCTTCTAAGAGGCAAGCAACAAAATAGAAT
AAAATACAGTAAGGTGAGATAAAAGGGAAACCATCATATTGAGGTTGGACAACAGAAACCAACAGAAAGATAAAAA
GATCCCGAAGAGAAGGCGCAGGAATCAGAGACCCGGATTCACAGTTCAGTCAGGAGTCCCATAAACGCACTCAACT
GAAAGTTAGAATACTGTGAGTTCTCAGCCTTCCCGATGCTGCCGCCCTTTAATACAGAACACTGAAGTGAACCCCA
GCCATAACATCATGTTCCTAGCTACTTCATCACGGCAGCTTTGCTACTGTTTTGAGTCATATTGTAAATATGTAAA
TATGTTTTCTGTTGGTCTTAGGCAACCACGGAAAGGGCCCTTTGACCCCAGGGTTGAGAACCGCTGCTATAGTACA
CATGCAGAGGACCTAGTGGAGACCCGTGCAGGCCCCACGCTTGCTGCTTCGGTCTCGGAGTTCATCGGAGCTTTGC
TCGGTTGTTTTAAGAGGACCTTGTTCTGATGTCCTCTGTCTCAGGTTTTTGCAACTCCTTCTGCCTCCTCTTTCTC
AGGGTTCCCTGAGCTCTGAGGGTCGGGATTCGATGGAGCCATCTTTTAGCAGTGCGACTAACCCACTTTTCATAGG
CATCGAATCAAACACTGCTTATTAATGGCCACTTCTCCTATATCTCTGGATTCTCCTTCTGAATGCGCAGACCGAG
CCTATCACAGGCCATTTGGACAGCATGGAAGGGCTATGGGCCTCTTGCACTTCCTCTTCCGGTTGCATTGCTTGAT
ACCACTTTCCGTGGCTTGGTCTGGTTTTACTCAAAGCGCACCAAAGCTTTGATCCAGTCGGAATGTTTCCCAAGGC
TTGCTCAGCGTGGGGGGATGGGAGGGTGTAGGTTTCAGAGGATGTGGCCATTTGTTGCAGATTTGCTTTTTGAACA
GGATTGCATGTTCAAAGACTCTTTATTGAACTATTTGAACTTCAGATATTTAAATGAAGGATTTGTAGGAATTACT
TGCTTCCTGGCTTACAGGGCTTTAATTAAGTTTCCCCCCCTCCAGTGTTGTTTCTAACTAACCAATGCGGCTTTGA
GATAAATATTTGAGAGTGTTTTTGTCATTAAGGACACTGGATGTTGAACTTCTCTGTTATTGACACATGGTCTCAT
ACAGTACAGGATGGCCTTGAACCTTTGATCTACCCTGCTTCTACCCTCAGTGCTAGCATTAAGCACTACAGTACCC
AGCCATGACAAATATTTTACATCCTCAGTAAACTCAAAATGCTTGTTAGGGTATCTGATATCAGATTATTGTTTCC
TTATTCTTCCATAACATTTAGCGAAGTCACTTAGAGGTTAAGCTAGCAGAGTGACATGCCTAAGGATGTATTATAC
TAGTGTGTGTGTCTCTCTGTGTATACACATGGGTTAATATTACAAAATGGTGCCCACGAGTTGCTGTTTATTCCCT
TTGTATTTGACCTGGAAATAGAGAGATGGCATTTAATTATAGAGGCTTAAGAAGAGGCTGTACGAACATGGAGAAT
CTACCTTTGGGGTGTTTTGAGGTTCTCACCTGGAGTAGCTGGGGCTGGTATCCCACCTAGCTTGGAGCCAGCTACT
CCTGCTATTGCTCTTTGGAGCATCCTGGGTTGCAGCTGTGACCTGGATGTTGCTGGGCGTAACCCCCACCAAAAAT
CCTAAGGTGGGAGAAATGTGAGCTTTGGCTGGGTGCAGGGTTTATGGTTGGATATAAAAGTCATTTGTATTTCTTA
ATATTTGTAAGCAAATGAAATCACTCCCCTTGTGCCTACCCACCTAATGGGACATTGCAGTAGATAAAGCTGGTTT
GTGTCCAACATTTGGAGAGGGTTGTGTAGGTTTCAAGAATCCAGAGTTGCTTATACTCAATGTGAGACCCTGTGCT
TCTCTCTGTAGAGTGCCTGCAGTCCGCCTGTAGCATGTGTGGCATCTGGGCCCTCTTCGGCAGCGATGACTGCCTT
TCCGTGCAGTGTCTGAGTGCGATGAAGATCGCGCACAGGGGGCCAGATGCATTTCGCTTTGAGAATGTCAATGGAT
ACACCAACTGCTGCTTTGGCTTTCACCGCTTGGCTGTGGTTGACCCGCTGTTTGGAATGCAGCCGATAAGAGTGAG
GAAATACCCTTATTTGTGGCTCTGTTACAATGGTGAAATCTACAACCACAAGGCGGTAAGCAGAGTGAAGCTGGGG
ATGGCTGGCTGGCTATAACTCACCTTCAGACCTATGGTTATGATGTTCATATTTTGTGTCACGCTTTTTTACTTTG
TATCTTTTTCTATGTTCTGACTCCTTTGCTCTGGTCTTATACAGAAAACTGACACAGAAAGGTAAAATTAGTTAGA
TTTTTTTTGAAGTGATTAAGATGGAGATGGGAATTGAATTTTTTTGGTGTTATAGATTTTAATATTGATTAGGGGA
GTGTTAGCTGATGGAGTCCTGTCAGCGAAAATGTCAGGCCTGTGCTCCATTGCAGGTTACGCTAACAGGAGACAGA
CAAAAAAATCACTGTAAATTGAACTTTCGGAAAGCAAATGCAGAAGTGGTGCACATAGGAAAGCGGTTTGCGGGGA
TGGGTCTGAGGAAGGGAGACTTAAACTGAGACCTGATAGACAGAGAATACTTGCCAAGCCCCGGACGTGTTTCCCA
TAGAGGGGGATAGCGTGTGTAGATGCCCAGTGTGGGCGAGAGTGAGTGAGAAGAGGACCCATGGAGCTGGGAAGAC
AGGACAGTCAGTCTCCTGCTATAGGAGCCTTCCAAGGCCAGGAGCAATTGAGGGCTTAGAGGGGAGAGATAGTCCT
GCAATGTTGAGAGGAGGTGAGATTTGCAGTGTTATATAATAGGGCCACTGAGAAAGCGTTTGCAGTTTCACACTTA
GTAGGAGCCAAATTGTAGAAAGGTGGCCGGAGGCACTGAAGTGTGTTAAGCAAGACTTGCAGCATTGAAACTCTCT
GGTGTCTAGAGAACAGGAGCGAGTGAGGGGCAGAAATGCTGATCTCGGAAACCAGATATGGTACGGACCATCCAAG
CCAGGATTATTTGACTTGTTTTTCATAAGGAAAAAGAATTAAATCTTTAATTTATGAGTGCTTGTACAAATACCAC
CCCTTGTTATATTTGCAAACTTGTTAACCCCTCCCCAACTTCTAGTTGCTTCAGAGCAATGTGCCTGAGTACTGGG
TATTGTTACTGCCTGTCTGTATTTATCTCCAGGCCTGCGTGGTCCCTTTAGGCTGAATAATTCTAGTATGGTACAC
CATAATGTGTTTCCTCTAAAATATGTAATCATTTGTCATTTAATAATCAGAACATTCTCCTAGCTAACGTTAATTC
CCAACCTAAGACTATAACATCTAATAATATAATAATAATATAATCTAATAATATAATATCTAATATCAAGTTCATA
GTTAAGCTTCTCCAAATGTCCCTAGAATACATTTAGCTGTGTTTTTGTTTGGCTTTGCCTTTTTGTTTTTATTTTT
TTGGTGTGTGTGTGTGTGTGTGTGTGTGTGTGTGTGTGTGTGTGTGTGTGTGTGTGTGTTTTCATGCTTATGCACC
CCTGTTTGGTTTTGTTTGTTTGGTAGGGCAGATTTCAGTCAAGGTTTAGTCATTTGGCCATGCTGTTTCATGGTAA
ATTTTGAGACTTCTGTGCCTATCTTTCATTGGTAGGGGTTTATCTTTATTCCTGGACAATGGTTTTAATTTGCCAG
TTTGCTTGCTTATAGGGGCATTGTCAAAGCAGGGTTCTCATCTGAAGCCTTTAACCACTTAGCAAATATACCTTCA
TAAAGGGCTTCTGTATGCAAAGCTCAATGCTCTAGGATGAACCCGGTACAGAGGAAAGTTAAGGAAAGCAGTGAGG
GGTGAGGTGGAGTTTGGGTCTCAGTAAGTCAAGCCAGTGTACCTGAGAGGATGAGGTTGCGCTCTGTCAGTATCAC
CCTTCATCTTGTCTTCCTGAAGTGAGGTTAGCACATATCTTGGCTTATGGTTTACTTGGACAGTGTTGCTGCTGCT
TATTAATTTTGTTAACAATTTAAAAGGCAATATCTTAAAAACATGCCTTTTAAAAATTAGGTAATGTTATATTTGC
AGAGACTTTCTGCTGTTTCTACCAAGCATCCAAACTTAAACAAATGCGACTGCTACAAACTTGTCTAAGAGTAGAA
TGCACAACTTCAGCCATAAGTATTTTTCAGAATGCACCTTCAAAGTTCGTCTTCACCTCCTGCTTATAAGCCAGGG
GCCCAGAACAAGTCCGAGGCCGGTGTGGCTTTAGGAAGGTGGCAGGACATGTGTGCAGCACAGAAGCGTCCATTCC
AGAGTTAGAGTTAGTACTCGGCAGTTGTTAGTGAAGGGACGAGTGCTTTCTGAGAAAATTATCATTGAAAATGAGA
GTGGAAGCGATGTTCATTATTTTCAAATGCTGTAGGGTGCTCCCACCCAGAGGGGTAGATTTTTGGGGTTGAAGAG
ATGCCTCAGCAGTTGAATCTGTTCTTCCAGAGGTGCTTAGTTCAATTCCCAGCACACACATGGTGGCTCGGGACCA
TCTAGAAAAGAAACTGAAGCATGTAGGTGTACACACTGCAGAACACTCACACATAAAATCAATAAAGTAAACAGAG
GGGTAGATTTTTCAAACCCCACCTACCTGAGCCAGGACGACCCTGACCAGTCCTTAGCTTGGAAGAATGGCCAGCC
GGTACTGTAAGAGAGGCAGCTAAAGCACGTGTTTTAACAGTTCTGTGAATGCTTTCCACAGAGGACCACGCGGTAG
GGATGGAAAGACCTAACGCGTGGCTGGTGTGCTAGTGTAGTAGAAACTGTCAGAATCTGGGCCCAACTCAAGCCTT
TTCACTTTGCTGACAGAGACAGTGGAGGGCCCATTGCTGTTCCTGTGCGGCCTATCTATGATCATTTTCTCTGTCC
CTGAGAAGTAGCTTTAATATCCCACTATTCAGGAAGGTCTGGCTGTTGTCCTCTCCTGAGGACCAGCAGCTTGGGT
CTGGGCTCTGCCGTTGTGATGGATTCCTTTGCATTTGTCAGTAAGGGGTGGCCACTAGGTTTTCCTTTTGCTGAAT
ATTTCAAGGGAGGTCTTTGAGGATTAGCTGGAAGGGCCTCTCAGCCGATTCTGCATCTCAAACCAAAAGTCAACAG
TTGTTGATAACACATTTTTGCTCCATGGTAACAAGGCAAACTGTTCTCCAAGTGTGTTGGCATGTCCTTATCTATA
TTCTTGGTTTCTACCTTCAAAGATCAGAGCAAACACTGGTGGGAAATGTTTATGAAAAATTATACCTATGATGAAC
ATGCCCATGTCTTTACACCCTGCACAATATTGCATAACAATTGCATCTAAAGTGTTGTATTAAGTTTTTTTTCCCC
CTTTGGTTTTTTTTCGAGACAGGGTTTCTCTGTATAGCCCTGGCTGTCCTGGAACTCACTTTGTAGACCAGGCTGG
CCTCGAGCTCAGAAATCCGCCTGTCTCTGCCTCCCAAGTGCTGAGATTAAAGGTGAGCGCCACCACTGCTTGGCGT
GTTAAGTATTCTAAATCTAGAAAGCATTTAAGTGCACAGGAGGATGTGTATTATATTATATGTAGATAATATTCTA
TTTCACAAAAAGCACTAGGTCACCTGCAGGTTTTTGTCTTGGGGCTGGGGTCCTAAAGAAAGGATGACTTCCCATG
TAAATGTGTATGTGTAACTACAGAGTGTAGACTCTGGGCTGCAATATGTACTTGAATTTCTTCATGTGTCACAGCT
CTGATGCCTGACCCTTGTATCCTGTTTATCAGAACACAGTCTTTATCGTCATGGGTTTAGCCTTTATGGTGAAAAT
TTTTCAGAATTGTGAGTTTAGACAAGGTAGATTTGAATAATGCCATACTTTTTTTTTTCTTTCTCCTTCTTCTTCT
TTTTTTTAAATGAGTAATGGAGAGGAGGTGATGGTAGAAGAGAGATAGTGGGTGAGGATGGATAAGGATAGTAAGT
TTTTGTTTGCTCTGCTTCCTTAAGCTACAGCAACGTTTTGAATTTGAATATCAGACCAATGTGGATGGTGAGATTA
TCCTCCACCTCTATGACAAAGGAGGCATCGAGAAAACCATCTGTATGCTGGACGGGGTGTTTGCATTCATCTTACT
GGACACTGCCAATAAGAAAGTATTTCTGGGCAGAGACACCTATGGAGTCAGGCCCTTGTTTAAAGCCATGACAGAA
GATGGGTTTCTGGCTGTGTGTTCAGAAGCTAAAGGTAATAATGGAGTTTAACTATTTTTGGTTTGTGTTTCTTCCT
GGATCATAGTGAGAATCCCAGAGAGGTGTCAATCAAAAACTTTAAAATGATGAAAGATGAATACTCAGTATGAATA
GGATTTGAAATCTTAGTCAATCCTTTCCTCTTTCATGTGTGCGTGTGTGTTGTATACACGTGTGCTCATACATACG
CACACGTAGAGACCAGAGACTTAACATTAGACATGGTAATTTTATTTTATTTAATCCGCCTTCCTCTGCCTCCTGA
GTACCAGGATTAAAGATATGCACCATCGTGCCAGTCTCCATTTTAATTCTTGACACGGTCTCTTTGCACTTAGAAC
TCACTCACTCACGTTCCTGATTTAGTGGCTGAGCTCCAGGTCTCCACCTGCCTTCTTCGTCCTGCCCCACCTATCC
TAGAAATAAAGATGCAGGATGCTGCTACCAGGTTCTCCATGGGGCCTGGGGATTAAATCTCAGTTCCTCAGGCTTG
CTTAGCATGGGGCTTGGCAGGCACCTTATTCACTCAGTCTTTCTGCAGCACCCGGTTCTTCTGGCCTCTAGGAGGT
GAGCACATTTTTTCTTCATGCAAAGTATCTCTGCCATGGTTTATAGATAGAGCCAAGCAACCACAGACTTGGTCAC
AGTGGCTCAAATGCTTGCGCTAAAATAATATTTCTTTTACATTTCTTATCTTGGGTATATTATTATAGCAATAGAT
ACCTTAACTTGTTATACTCAGTATAATACACCTACTTAGCATTATTTTTCTGGCAGTAAACTTTGTAATCATCTTT
GGTCTCTTACCTAGATAGCAAGTTTTCTGTAGTGTAGTTTGCCATTTTTTGAATCCTTTGCCACTTAGTCTTCAGA
TTTTACATTACTTGAAACCAGGATCTTGATCTGCATGCTAACCAAAGTGTGGGTATTAAATATGCTTCCCTTGGTC
TGTCAGCAGACAGCATAGGGCATTCTACATCAGTTACCTAAAATCCCGTCAGGGAAACCTGGCAAAGGAAATATAA
AGCAGGATGGACTCCTGCTTCTGCGGGCTATCTTTTGGACTTTTAGACCTGATGATGGCTTGATACGGACCTGGAA
TCCCACCAGCAGTTTACTCTCTGCAGTTTGTTTTCTGCATCCTTAACTCTGTCCTTTTCTAGACTGCCTAGCGACC
TTTACATTACTATGATAAGAAATCACGACCAAGACAAGACTTTATTTGGAGCTTAGAGTTTCAGAGGGCTAGGGTC
TATGAGGTCATGATGGGGAACATGGAAGCAGGCAGGCAGCGGCTGGGAGCTTACATCTTTTTTTTTTTTTTAAAGA
TTTATTTATTTATTTATTATATGTAAGTACACTGTAGCTGTCCTCAGACATTCCAGTCAGATCTCATTACAGATGG
TTGTGAGCCACCATGTGGTTGCTGGGATTTGAACTCCTGACCTTCGGAAGAGCAGTCGGGTGCTCTTACCCACTGA
GCCATCTCACCAGCCCCCGGGAGCTTACATCTTGATCCACAAGCATGAGGCAGAGAGCTGACTGGGAATGATGCGG
GCTTTTGAAACCTCAAAGCTTATTCCTAGTGATGCCCCTGTAAGAAGGCCACACTTCCCAATCCTTCCCAAACAGT
TCTATTGACTGGGGGCCAAGTGGATATGCTCTGTAGGGGTCCTATTGAATCCACCACACAGATCCCATTGGAGTGC
TGCTTCCTTAACCAGGCTTTAAAACCATCTCATCGTGGTGATCTGCCTAAGACCTTTCAGTTGGCTGCATCACATC
CTCTATGGGGTGAACAGCATTTGAGCTCTGCCAGCTAGTGAGCACACTGCATTCCTTTGACCTTTGAATTCAATGT
ACACAGATTGGGTTATAAGACTCATAGTACATTATAACAAATATCTGTGTAGATAGCCTGTTTATCTATTTATTAC
ACAACATTGGCTTTTGTGCAGATCACGGTGGTGTCAGGGTAGCTACTGTCTTTAAGAACTGCCTACAGTTACATTC
TATGGCTTTCTCATACACATTCTATGACAGCCATGAAGGGGTGGGTACTGGCTTCTGAGTACCAGTAGTGTATCAT
TTTACGTGGTAAAGACTTGAGAACCATTGGTCTCTCAAAATACCATGCCAGTTAACATGAAGTTAACATTCTGATT
TCTCTTTTTTCTCAGATTTATATTTCCTCTTGTTTATGTTCTGACAGAAAATAGGCCTTCCCTTTAAAAAGCTTTT
TTCTTTGAAAACTTTATAAATGTCTAAAAGACAGGTTTCAGAAAATTCAGCTAGGTAAAGGAATGGGATGATGAAG
ACTTTCATTTTAATAAAGGTATCAGGGCCTTTCAGGTGGGAGAGAGGATTGTCTCAGAGGATGTTATTGTAACGAC
GAGACATACACTAGTTAAAAGGTGATGTTTGGAATTTTTATCTTGCTATGTTAGGAAATTATCTCGTAAGGAATCA
TCTTCCTATTGCATCAAATGACTGCAAATCCATGCTGTTTTGTGTTCTGTCTCCATGTCATTCTTGCTTCTTCAGG
AGAGATGCTAGGGTATGGGGTAATTATTTCCCCTCCCACCTTTTTTGTATAATGGATATGCGTTTGTACTTAAGGG
GGAAAGTATGGGTTAGAGTGTACTAGCTCACTGCACAGGCTGCAGAGAAGCACCTAATTGCATACGTTGCTGTTTT
CCATTTATAGGCCTTGTTTCCTTAAAACACTCCACCACTCCCTTCTTAAAAGTGGAGCCCTTCCTTCCTGGACACT
ATGAAGTTTTGGATTTAAAACCAAATGGCAAAGTTGCGTCTGTGGAAATGGTCAAATACCATCACTGTACGGATGA
ACCATTGCATGCCATCTATGACAGCGTGGAGAAACTCTTCCCAGGTGACTAGTGCCCATGACCAAACCCCAGAACT
CTAGCTCACCCCTGACAACTCAGGGCCATTTGCGCCGTTTTCAATGATGTTGGTTTTAAGATGTTCCTCTTTGATT
TGGAGGTGGAGCGATCTCCGTGAGTGAGGGAGAGCCATCACAATGATGTAGTAGACTGCTTGCCTCATCTCAGTCA
GCTTAGCCTCAGCTTGTTTGTGGCGGTTCCATTTACTTTCTGTGTTGTTAGGCTTTGACCTAGAGACCGTGAAGAA
CAATCTGCGTATCCTTTTTGACAACGCTATCAAGAAACGCTTGATGACAGACCGGAGGATTGGCTGCCTTTTATCA
GGTGAAGTCACTTTAAAAAATAAAAACATCTTACTGGTGTTTAAATGAAATGATTTGTAGCTGGCACTGAGCTTTG
AATGTTAAAGGCCTACTATATAGTTTCATGTTTCATTTTCTATAGACGTGTTTATAAACTGCATTTGTTAGGGTTA
GAGCTTGTTGGTCCTCATCCCCTAGACTCCTGTGTATTGATCTTTTAAGCCAGTTTAGATGCAGTTGCCATTGTAA
GGGGACAAGAAAATGTATGTATTCACACACACATACACATACATATACATATGTATGTGTACATACATACACACAT
ACATATATATATATATATATATATATATATATATATATATATACATACACACACACACAGACACATACATATATAC
ATACATACACACACACTTCATTTCACTTCACTATAAATCTTACTAAAGCTGATTTCTGTTTATAAAACTCAGAGAT
CTTCATAGTCAAATGCCAATCCAGATGTTTAGCAGGTGTGTGATTATCTTTGCCCTGATTAATGTATGCCTGCAAC
CATCAAGTTTATTTTCATGCTGCAGGGGGCCTGGACTCGAGCTTGGTTGCTGCCTCTCTGCTGAAGCAACTCAAGG
AGGCCCAAGTTCAGTATCCTCTCCAGACATTTGCTATTGGCATGGAGGACAGCCCCGATCTCCTGGCCGCTAGAAA
GGTACAGCACAACCTGCCCATCGTATAGACTCAGAACTTAGCCCAATTGTTAGGACGAAAATCAAGCTGAAGTCGT
CCTCGTGTGCCTGTTGTGTTACTATGAGAAAGGTCATGCTAGAAAGCAACAGGGTGAGGAACGGATTCACGTGGCT
TACATATCCCGAGTTGTAGTCCACTGACGAAATCTAGGTGGGAATTTGAGGCAGAAGCCTGTAGGCAGGAACTGCA
GCAGAGACCATGGAGGGGCACTGCTTAGTTGTTGGTTTGCTCCTCCTGGCTCCTGATCCTTCATTTCCCATCTTGA
GTTTTCTGCTTTGTCTTTCCTCCGTGGACTGTGACTCAGATAAACCAAATAAACTCTTTCCTTTCCAAGTTGATGT
TGGTCACGATCTTTGTGACAGACAAGCCATAGAGAACAACTAGGATAGGGACTGGTGCCAGGATATCAGCTCGCAT
CTTGTTTTCTTATGAAGCATATGCATTACAAGCAGATATTGTACATGCAATGCCGTGTGTGGAAGATGGCGTGCTG
CTCTGTAAAGACCTAGAATGACTGAGAGAAGCCTCGAGGAGAATTCCAATTCGACAGGAAAAGGCTATATATGTAG
GGATACCATGCTGGATATAAAATTAGGCATTTCAGTACAGTACAAGGGAGATGATGGTCAAAAGGGAGAAATGAGG
CAGGTTAAGTAAAATAGGAGAGAGTGGTGTGCTGATTGGGACTCTGGACTGTCAGTCTGTAGCTTTGATTTGAAGA
GAGCCAATGTGGTTGAGTGGGGAGTGAGGGCCAGGCAGAGGCACCTGGCTTGCTTTAGGTAGCATGTGGCTGTGCC
TGCATCTATCTCCTCTTACAGAAGGGGCCTGCACCTCCTCTTATTGAAAGGCCTGCACCGCCCCTTACCAAGGCGG
TCTGGAGAGACCTTGAGTCATCTTCAGCTTTGTTTCTTTTCACCATGTTTACCTTGAGAGGAACTGGCCTGTTTCA
TTTTCTAAAGTGGTTCTTTAACAAATCTATGGAGTTTTTCTTCACAATCAGGTGGCAAATTATATTGGAAGCGAGC
ATCATGAAGTCCTTTTTAACTCTGAAGAAGGCATTCAGGCCCTGGATGAAGTCATATTTTCCTTGGAAACTTATGA
TATTACGACAGTTCGGGCATCTGTGGGTAAGGGATTTTATTATAGGGAAAAGCGTTTTAATACTGAAACAGTAGAA
GGTTTAACATGATTTCAGCTTAAGAAATATTGTCTAAATATGAATACTAGCAAAGTTCCTATATTGTTAATAATAC
TATAATATAGTGATAAGACCATTTAAAAGCGCCCTGAATTTCTTTCGACTGGAGGAAGTGCTTAATGAATATAATC
AGATCATCTGAACACTGAATTTACAAGGTCAGCTCGGCCTGTGGTCTTAAAGAATGCTTCATAAAAATAAAAGCAG
CTCACATCTGCTAAGCACATACTTCATGCTAGGTTCTGTCTCCCATACTTCATATGAATTATTTGTTACCTCAGAA
CTAACTCATCATAACCAGGATTCCAGCACCCAGGAGGTTGAGGCAGGGGGATTGCTGGGCTGTCAGCATATGGTAT
GACCATAGAATGGACACATATTTTGGCTCCCTTTCTCTCATCTTCTCTGTTTAAAATATCTTGGATGATATAGTTG
GGCCACAGTACATAACATTATTATGTATAATAATCTGGTGTGCTTCCTAGTGCATGTAAAAGCTATGGCTACAGTG
TACTATGGTCAACTAAGTACCTTTATTTAGGAATATCTTATTGTTGTTGATCATCTAAGCTGTGGAAAATGATACA
GTGTATTTGCTTAGTGCAGGATTGCCAGACTTTTCATTTGTAAGAAATTAACTTCCTCTGAGATACAATAAAGCTA
AGCATAGTAAAACAAGATAGGCCTGTAGATGTGTGTAGGTTGTGTTCAAATGCTATCATTTTACAAGGAGTTGGGT
ATCTGAAGATATGTGTAGCTGTTTGGTTCCCAGAACCAATCCCAAATGAATAATGAGGGAACACTGTTCTTAATAA
AATGGGGAAATCGGTCCATGTTACAGTACATTTTCTCTAACGGGTCCTCATGTAGATGTATAATAAGTATTCCATC
ACACAGCTTACCAGTTACTAGCTATTATTGTCATGCAAGCAACACTACTTCTATCTGGCGAACACATTCTAAGATT
AGAAATGTAACACAAATCAGACCCAATTTTGTTGAACCTCTAAAATTTTACTAGCTGAGTGAGTCCTTTTAAAAAC
TCACTTTGGAGTGTGATAAAATACCTTGGGTATTACAGTGATTTGGTCTCCTGAAGCATCATAGTTAAATACTCAC
ACGTATAGCACATCTGTGGTTAAATTTTTATGAGGTGGAAGCAGAGTTAGAAGAATGTCTCTCCTAAGAAGGATAA
AAATGAGATTGGAGGAGACAGATGGTAGTGTAGAGAGATGGTAGTGTAGAGCTATCTACCAAGTAGACATAGCACA
ATATTGTACAGAGCCTTCTTAATCAAATGTGGGATTTGGAAATGAGGGGTGGAAATGGAAAAGAACGAAACAATTA
AAAGTAAGCTATTTCCATTAGTCTGCTTTCTGTTACAATAATGGATAATGCCAGATGAGAAACTTTAGAAAGCCCA
ACAGCATGGCATGTTGGTGGGTGGCATCAGGAGTGGCACATGAGAAAGGCATCTCATAGTGATCCCAGGCATTGGA
GACAAGAGATGGGCTCTGTCCTAAATAATTGAATCAGGAGAAATCATTTGATGCTTGAGGAGCAAGGAACCCCAAC
CACACCTTATCTTCCACTTCTCCACCATGGATCACACCACCACATGGAGACCAAACTGCATCCAAAGCATTGCAAG
GCCACGCCTTACTCTACTGTGTTAGTACACATGCCGTGGTATTCAGGCATTTGAGTGGTTCAATCCAGCAAACATT
TAACATAGAAAGTCTTGTTCTAAGAACATAGCTTTGCAACCTGTTTGTAAAACATGACAGTGGATTGCAGGCTATT
TTATTGTTTAGGTTGGATTGACAGCTTGTAGATGACTTACAAATTTCCGATCCTTTTGGAAGCTGCTTTAATACTT
ATACTAACTCAGTCCAACAAGACACTCCAGCCTACTGTTGCTTCTGAAGACAACAATGACATCGCCATTGATTTCT
CTTTCATTTAAGGCATGTATTTAATTTCCAAGTATATTCGGAAGAACACAGACAGCGTGGTGATCTTCTCCGGAGA
GGGGTCAGATGAACTTACACAGGGCTATATATATTTCCACAAGGTAAGCGCTCTGAACTGAAACGATGGCCAGGCT
AAGTTAGGAAGTCAGAATCACTTGACCCTTTCTATTCTGTCCTTATGATTTGTTGTAGTTTTAGAGTGTATTTGTG
TTTGTGTTTAAGAATTGATGCTATCTATCTGTCTGTCTGTCCGTCCGTCCGTCTGTCTATCTATATATTTATAGGA
GAAAGAAGACTTAATAGTATCAGATACTTTTTATTCTTAAACACGAGAATTGCTTTTGTATTTTATCTGTCTGACT
GTCTGTCTATTGATTTTTTTTTTTCAAGACAGGGTCTCATTAAGTAGCTCTGGTTGTTGAGAAACTCACTTTATAG
ACCAGACTGGCCTCTGCTTCCCAAGTGCTGGGATGCAAGGCACTGTGTCTGTTGCCCAGGGCACTCATTTAATTTG
TTTTCACACTAAGCACTTCGGAGGATGGATGGGTCGGTCTTGTAAAACATTGCTGGCCTCTGAAGTCATTTACTCA
CATCCTATACCCTCCTCTTAAGCTTTCTGCCTTACTCATTCTTTATTTCATATGTAAGAGGGGAAAATGAAGGCTG
CCTGTGTGCCAAAGGAAGGCACTCACCTTCGTTTGTTAAATAGGCTCCTTCCCCTGAGAAGGCCGAGGAGGAGAGT
GAGAGACTGCTGAAGGAACTCTACCTGTTTGATGTTCTCCGGGCCGACCGCACTACTGCTGCACACGGGTAACTTT
AAAATCAGTTGTAAAACCAACAAGGGAGAAGGGGGAAGTAGCTTAAATGAGATCAGTTGCTTATTTTGAGTTTCTT
TCGGCTGGCTGCAAAGTATGGTTTTCAGCAAAGTTTGTCCCCTAAAAGCTGTGCTATGGCCTGGACACTATCACCG
GGGTGTGGAGAGATCATGTCCTGTGGGTATGCCAGGCAGGTTTTGTGTCACTAGATCATAACAGCAATGGGCCGTT
TCTGGCCTAGAGGGAAGGCTGCAGCGGCTTCTTGGAAACATTTTAGAGCATTTGGAGTATTGCTTCTGGTAGCAAA
TACTACGTGCCTGCCAAGCCAGTTTGCTGCATCTGGGAGGAGAAATGCAATCTTCATGCATTCATTCATTTGTTGA
TAGGACTATAGAATAAAAATAAGTTTCTTTGGAGCCATTTCTTCTTGCCCAGGGTTTCTAGGGTTAACTGGGCTTG
TTGAAGAATAGAAGAGCTTTACTGTGCAGGATAAAGACTTGCCTTTCAACATTTTCAGTTCCTCTACTGAACCATA
TTTGGCTTACGAATGGTCTGGAATCATAGTTATGACCTGCCTGCATGTCCTGACGAGTGTCACTGGGACTTTTGTA
CATATGATGGTATGAATATTCATATACCCTCATGACCAGGTCACTCATGATAATTAACGTTTTATAGAAACAGGTA
ATTTTTCATAGGCGCAGAAGGAAAGAAATACTTTAAGACAGAAAGCTTCCCTCCCTAGATGCTGGTTTTATTTCAA
GTACAAACATTAGTATGTGGCTAATCATGCAGTGTGGTCCCTAGGAGGCTGGAATGTAACTCTTTCTTCTCCATGA
CTCAGTCTGGGGAGGTTGTCAGGAAGCCGTGCCAGCCATCTGACTCAGATTGTCTTCTGTTTTCCCCAAGTCTCGA
ACTGAGAGTCCCCTTTCTGGATCATCGGTTTTCTTCCTATTACCTGTCTCTGCCGCCAGATATGAGAATTCCAAAA
GTAGGTATATCAACCCTGGATAAAACCCCCAAATATTGTTGGTTCCTCCGGCGACTTCACTGACCCTCCTTGTGTG
CTCCTGTCAGAATGGCATAGAAAAACATCTCCTGAGAGAGACTTTTGAGGACTGCAACCTGCTACCCAAAGAGATT
CTCTGGCGACCCAAAGAAGCCTTCAGTGATGGGATCACCTCAGTCAAGAACTCCTGGTTCAAGATTTTGCAGGACT
ATGTTGAACATCAGGTCTGTCTTTAAAGATATATATTCTGGAACAATAATAGTTTAATAGAGAGCTGGGATCTCTG
TTTGCCTTTCGTTTGAATAGTTGTATCTAAAGGAGAGTCAGAGTTCCTGCAAGGAATATGTCTTTTATAAGCTTTA
TGTAATAATTAAGGAAAATGAAGGACTATAAAGTAGTATTGATGATCAAAAGAAAAGGGATAGAGGGAAGACTTCC
AATTCATTGCTGATCATTTTGCACGTCTGGGTACCTGATGAGTACAAATGAGTCAAACACTGAGCCTCTAAGTATT
AATATGCATGAGAGCTTTTAAAAAGTCACTGTCTGTTTATTATGGCTTGCTTTGGCACTTTCTAAATAATGAGTGA
CGTAAAGGGTCTTGTTGCTTCATAGCATGCCTTAACTTTTTTTTGGTCACCATCAGAGCAAATACACATCGAAATG
TTTTATGTTCCTACTTTATCTTATAGGTTGATGATGAGATGATGTCTGCAGCCTCCCAGAAGTTTCCCTTCAATAC
TCCCAAAACTAAGGAAGGCTACTTCTACCGTCAGATCTTTGAACGCCATTACCCAGGCCGGGCTGATTGGCTGACT
CATTATTGGATGCCTAAGTGGATCAATGCTACTGACCCTTCTGCCCGCACTCTGACCCATTATAAGTCAGCTGCCA
AAGCTTAGGCACTCTCTACACTCTTGTGTAAAAGTAAATGTTTCTTCCGGCTCTGAAGGTCGAGACAGCGACACAA
TCAGAAAGAATGAGACTCAGCCATCAGTCACCCAGGCTTACTTAGGCATGAAAAGAAATAAAAGTTTCACATCTGA
AATGCC

### LNA Transfection

For silencing with LNAs cells were seeded into a 6-well plate, 140,000/well in 10% FBS (Gibco) in DMEM (Gibco) without antibiotics. Transient transfections using Lipofectamine RNAiMAX (ThermoFisher) was performed according to the manufacturer's instructions. Cells were cultured for 24 or 48 h after transfection. LNAs final concentration 50 nM. After 24 and 48 h, cells were trypsinized and counted with an automated cell counter (Countess cell counter, Invitrogen). Picutres were taken with a white field microscope, using a ×10 objective.

### Real-Time PCR Analysis

RNA was isolated from plated cells or snap-frozen kidneys using the RNAspin Mini kit (GE Healthcare). Total RNA was isolated using the RNA-Isol lysis reagent according to the manufacturer's instructions. For reverse transcription of RNA, Oligo (dT) 15 primers (Promega) and ImProm-II Reverse Transcriptase (Promega) were used. Quantitative real-time PCR analysis was performed on duplicate using SYBR Green I master mix (Bio-Rad) on CFX96 Touch Real-Time PCR Instrument (Bio-Rad). Primers sequences:

| | Forward | Reverse |
|---|---|---|
| Asns | GGTTTTCTCGATGCCTCCTT | TGTGGCTCTGTTACAATGGTG |
| | SEQ ID NO: 27 | SEQ ID NO: 28 |
| Malat1 | GAGTTCTAATTCTTTTTACT | AGAGCAGAGCAGCGTAGAGC |
| | GCTCAATC SEQ ID NO: | SEQ ID NO: 30 |
| | 29 | |
| Hprt | CACAGGACTAGAACACCTGC | GCTGGTGAAAAGGACCTCT |
| | SEQ ID NO: 31 | SEQ ID NO: 32 |

### Mouse Lines

C57/BL6 TmCre Pkd1^{ΔC/+} was bred with C57/BL6 Pkd1^{flov/flox} mice to produce the mice used in these studies⁴². Cre recombinase activity was induced by a single injection of Tamoxifen at P23/P25. Tamoxifen (#T5648; 10 mg/40 g; Sigma-Aldrich) was freshly prepared and dissolved in corn oil (#C8267; Sigma-Aldrich) by continuous shaking at 37° C. for at least 4 hours and was injected intraperitoneally. For treatments, once a week i.p. injection at 50 mg/kg/week for 5 weeks and then alternative week for another 2 weeks. Animal care and experimental protocols, approved IACUC 548 by the Institutional Care and Use Ethical Committee at the San Raffaele Scientific Institute and f approved by the Italian Ministry of Health. All mice were randomized for each experiment.

### Cell Lines

Immortalized Pkd1^{+/+} and Pkd1^{-/-} MEFs³⁶ and mCCD epithelial cells⁴³ generated with CRISPR-Cas9 technology for the Pkd1 gene (or empty sequence #CAS9RFP-1EA for control clones) provided by Sigma-Aldrich.

### Biochemistry Serum Analysis

Analysis of serum samples was assessed by ILab Aries, a bench-top analyzer that can perform photometry, turbidimetry, and potentiometry tests (Instrumentation Laboratory, Werfen Group, Milan, Italy). BUN was detected using kits and controls supplied by ILab Aries. Standard controls were run before each determination to monitor the precision throughout the experiment, and the values obtained for controls were always within the expected ranges.

### Immunohistochemistry

For immunohistochemistry (IHC), formalin-fixed paraffin-embedded consecutive sections (4 μm) were dewaxed and hydrated through graded decrease alcohol series. Stained for anti Ki67 antibody and anti-cytokeratin antibody as per manufacturer instructions. Antigen unmasking with Citrate Buffer pH 6.0, any endogenous peroxidase activity was quenched with 3% peroxidase water for 20 min at Room Temperature (RT). Antibodies dilution of 1:400, 1 hr RT, and developed with Rat on Mouse HRP-Polymer (Biocare Medical) or EXPOSE Rabbit specific HRP/DAB detection IHC kit (abcam). Immunostaining, DAB substrate chromogen was applied to sections for 5 min at RT and counterstained with Mayer's hematoxylin, dehydrated and mounted with Eukitt (BioOptica). External positive and negative controls were run simultaneously. Images were acquired using a Zeiss AxioImager M2m with AxioCam MRc5.

### Histology

After euthanasia, the inventors removed the kidneys, washed them in PBS, weighed them and fixed them in 4% paraformaldehyde (PFA). After incubation in a sucrose-in-PBS gradient scale from 10% to 30%, the inventors incubated the samples in 10% glycerol (Sigma) in a mixture of optimal cutting temperature medium (OCT) (BIO-OPTICA) and 30% sucrose and then embedded them in OCT. The inventors air dried cryostat sections for 1 h, rehydrated them in PBS, incubated them in 1:10 Harris Hematoxylin (Sigma-Aldrich) for 2 min, washed them, incubated them in Eosin G (BIO-OPTICA) for 7 min, and then washed and dehydrated them and mounted them in DEPEX (Sigma).

### Statistical Analysis

For statistical analysis the Prism 5, GraphPad Software, was used for statistical analysis tool. t-test was used for all analysis of two groups. ANOVA statistical analysis followed by Bonferroni's multiple comparison test was performed in all analysis where more than two groups were present. Paired t-test was done for intra-litters treated animals.

### Litters

4 litters were collected each containing intra-litters treated animals and controls animals. Litter 1,2, and 3, animals were treated for a weekly injection of a total of 7 per week. Litter 4, animals were treated for a weekly injection of a total of 6 per week. As show in the schematic FIG. 19
Litter 1:4 animals, males sacrificed at P94
Litter 2:4 animals, females sacrificed at P94
Litter 3:4 animals, females sacrificed at P94
Litter 4:4 animals, males sacrificed at P88

### Example 1

### Metabolomic Profiling Reveals Profound Metabolic Changes in Pkd1-Mutant Kidneys and MEFs

To gather a comprehensive picture of the metabolic derangements observed in PKD mouse models, the inventors applied non-targeted global metabolomics⁴⁴ to an orthologous mouse model of PKD carrying inactivation of the Pkd1 gene exclusively in the kidney as to avoid confounding effects derived from extra-renal inactivation. To this end the inventors employed KspCre;Pkd1^{flox/-} kidneys carrying inactivation of the Pkd1 gene in the distal tubules and collecting ducts of the kidney. To minimize phenotype variability in the experimental design the inventors used a pure C57BL/6N background (i.e. >10 backcrosses) and performed the study upon precise timing of the day of birth of the animals (see methods). Furthermore, samples were collected at P4, when the kidneys are already cystic, but not yet functionally or structurally severely compromised (FIGS. 7A and B). Importantly, neither infiltration nor fibrosis could be detected at this time (FIG. 7A). To further strengthen the outcome, the inventors designed the study so that kidneys were collected from 4 litters containing each 2 cystic (KspCre; Pkd1^{flox/-}) and 2 control littermate controls (KspCre; Pkd1^{flox/+} or Pkd1^{flox/+}, used interchangeably) of each gender (8 males and 8 females in total) (FIG. 1A) to maximise the possibility to use intra-litter controls. Kidney over body weight ratios of Pkd1-mutant mice were significantly different from controls, but very similar in male and female at this neonatal stage (P4), possibly because sexual maturation is not yet achieved (FIG. 1B). Furthermore, no increase in EPO or VEGF transcription could be detected, thus excluding the possibility of these kidneys being hypoxic (FIG. 7C). Application of Liquid Chromatography-Mass Spectrometry (LC-MS) resulted in the detection of 550 metabolites. A Principal Component Analysis (PCA) showed a clear separation between the cystic and control samples indicating a negligible influence of inter-gender and inter-litter differences in these samples (FIG. 1C). In line with this, Hierarchical clustering analysis (HCA) showed separation of the cystic and control samples (FIG. 1D). Paired t-test analysis was applied to take into account the intra-litter samples, resulting in the identification of 488 metabolites that significantly changed (adjusted p value, p<0.05), and 384 significantly different metabolites when considering both p-value and fold-change (adjusted p value<0.05, absolute fold change >2) between cystic with control samples. A volcano plot of the data shows that 213 of these metabolites are down-regulated, while 171 are up-regulated, and that the main alterations are related to amino acids, carbohydrates and lipids (FIG. 7D).

A number of different metabolic pathways were found significantly altered (FIG. 7D). Among these, striking differences were observed in metabolic pathways involved in bioenergetics, including glycolysis (GLY), pentose phosphate pathway (PPP), the tricarboxylic acid (TCA) cycle, fatty acid oxidation (FAO) and fatty acid biosynthesis (FAS) (FIG. 1E). The metabolites that appeared to be mostly accumulated in the cystic kidneys were citrate, aconitate and α-ketoglutarate (α-KG) (FIG. 1F). In line with this, KEGG-Pathways Based Enrichment Analysis showed that the TCA cycle is among the pathways affected with a statistically significant Enrichment Score(ES) (p≤0.05) (FIG. 7E).

These data indicate that the loss of Pkd1 leads to profound metabolic changes, broader than what previously appreciated.

The inventors then used a set of Pkd1^{+/+} and Pkd1^{-/-} Mouse Embryonic Fibroblasts (MEFs)³⁶ to further investigate the metabolic changes in Pkd1-mutant cells. A metabolomic profiling revealed that Pkd1^{+/+} and Pkd1^{-/-} MEFs are well separated by PCA with many metabolites being significantly different between the two genotypes (FIGS. 8A and B) and that metabolic pathways such as glycolysis, PPP, FAS, and FAO were impaired Table II (FIG. 8C).

TABLE II
List of the metabolites detected from MEF:

gamma-glutamylglycine
gamma-glutamylleucine
gamma-glutamylisoleucine*
ophthalmate
myo-inositol
acetylcarnitine
UDP-N-acetylglucosamine
guanine
gamma-glutamylalanine
gamma-glutamylvaline
gamma-aminobutyrate (GABA)
glycerophosphoethanolamine
hypotaurine
sphinganine
heme
isovalerylcarnitine
glycerophosphorylcholine (GPC)
N-acetylglutamate
phytosphingosine
pyroglutamine*
sphingosine
1,2-dioleoyl-GPG (18:1/18:1)
gamma-glutamylmethionine
glycosyl-N-palmitoyl-sphingosine
cytidine
1,2-dioleoyl-GPI (18:1/18:1)
gamma-glutamylglutamine
uracil
thiamin (Vitamin B1)
3-hydroxy-3-methylglutarate
xanthosine 5′-monophosphate (xmp)
N-acetylserine
pyridoxine (Vitamin B6)
cysteinylglycine
1-(1-enyl-palmitoyl)-2-oleoyl-GPE (P-16:0/18:l)*
pro-hydroxy-pro
glutamate
imidazole propionate
spermine
oleoylcarnitine
aspartate
orotidine
gamma-glutamylglutamate
1-oleoyl-GPS (18:1)
pyridoxal
palmitoyl dihydrosphingomyelin (d18:0/16:0)*
carnitine
4-guanidinobutanoate
1-stearoyl-2-oleoyl-GPS (18:0/18:1)
1-oleoyl-GPI (18:1)*
retinol (Vitamin A)
orotate
carnosine
palmitoylcarnitine
myristoylcarnitine
deoxycarnitine
propionylcarnitine
pyridoxamine phosphate
coenzyme A
creatine
glutathione, reduced (GSH)
erythronate*
nicotinamide
N-acetylleucine
2-methylbutyrylcarnitine (C5)
ribose 1-phosphate
2-hydroxyglutarate
1-arachidonoyl-GPI (20:4)*
1-stearoyl-GPI (18:0)
phosphoethanolamine
pterin
2-methylcitrate/homocitrate
1-linoleoyl-GPI (18:2)*
3-methylcytidine
1-oleoyl-2-linoleoyl-GPE (18:1/18:2)*
7-methylguanine
beta-alanine
1-(1-enyl-stearoyl)-2-linoleoyl-GPE (P-18:0/18:2)*
4-hydroxybutyrate (GHB)
arabitol/xylitol
1-stearoyl-GPC (18:0)
pyridoxamine
1-palmitoyl-2-arachidonoyl-GPE (16:0/20:4)*
homocysteine
N-acetyl-glucosamine 1-phosphate
stearoylcarnitine
2-palmitoyl-GPC (16:0)*
proline
glucuronate
1-oleoyl-GPG (18:1)*
1-oleoyl-GPC (18:1)
N-acetylthreonine
adenosine 3′,5′-diphosphate
1-palmitoleoyl-GPC (16:1)*
glycerophosphoglycerol
gamma-glutamylthreonine*
tryptophan
3-hydroxybutyrylcarnitine (2)
thiamin monophosphate
choline
S-carboxymethyl-L-cysteine
N-acetylmethionine sulfoxide
3-phosphoglycerate
1,2-dioleoyl-GPC (18:1/18:1)*
ergothioneine
1-palmitoyl-GPI (16:0)*
cytidine diphosphate
Isobar: fructose 1,6-diphosphate, glucose 1,6-diphosphate,
myo-inositol 1,4 or 1,3-diphosphate
N-acetyltyrosine
thiamin diphosphate
pyridoxal phosphate
1-palmitoyl-2-oleoyl-GPG (16:0/18:1)
3′-dephosphocoenzyme A
gamma-glutamylphenylalanine
carboxyethyl-GABA
UDP-galactose
1-arachidonoyl-GPC (20:4n6)*
serine
1-dihomo-linolenylglycerol (20:3)
guanosine
octanoylcarnitine
hypoxanthine
stearate (18:0)
1-palmitoyl-GPC (16:0)
N-acetylputrescine
N1-methyladenosine
N-acetylmethionine
1-stearoyl-2-arachidonoyl-GPI (18:0/20:4)
glycine
imidazole lactate
xanthine
1-linoleoyl-GPC (18:2)
gulonic acid*
1-(1-enyl-stearoyl)-GPE (P-18:0)*
UDP-N-acetylgalactosamine
guanosine 5′-diphosphate (GDP)
1-palmitoyl-2-linoleoyl-GPE (16:0/18:2)
nonadecanoate (19:0)
3-hydroxylaurate
diadenosine triphosphate
1-palmitoleoyl-2-linoleoyl-GPC (16:1/18:2)*
1-methylnicotinamide
UDP-glucose
1-stearoyl-2-oleoyl-GPC (18:0/18:1)
arachidate (20:0)
N-acetylaspartate (NAA)
behenate (22:0)*
N-glycolylneuraminate
1-palmitoyl-GPS (16:0)*
13-HODE + 9-HODE
phosphoenolpyruvate (PEP)
cystathionine
cytidine-5′-diphosphoethanolamine
3-hydroxybutyrylcarnitine (1)
2′-deoxycytidine 5-monophosphate
sedoheptulose-7-phosphate
1-palmitoyl-2-arachidonoyl-GPC (16:0/20:4)
sphingomyelin (d18:1/18:1, d18:2/18:0)
S-adenosylmethionine (SAM)
arginine
4-imidazoleacetate
acetylphosphate
1-arachidonylglycerol (20:4)
5-phosphoribosyl diphosphate (PRPP)
nicotinamide ribonucleotide (NMN)
asparagine
1-palmitoyl-GPG (16:0)*
1-oleoylglycerol (18:1)
2′-deoxycytidine
inosine 5′-monophosphate (IMP)
6-keto prostaglandin F1alpha
creatinine
flavin mononucleotide (FMN)
myristoyl CoA
glycerol
C-glycosyltryptophan
myristoleate (14:1n5)
1-methylimidazoleacetate
1-(1-enyl-palmitoyl)-GPC (P-16:0)*
1-linoleoylglycerol (18:2)
1-arachidonoyl-GPE (20:4n6)*
phenylalanylalanine
hexanoylcarnitine
1,2-dioleoyl-GPE (18:1/18:1)
1-stearoyl-GPE (18:0)
thymine
1-linolenoylglycerol (18:3)
ornithine
gamma-glutamylcysteine
glycosyl-N-stearoyl-sphingosine
N-acetylglucosamine/N-acetylgalactosamine
N6-succinyladenosine
cytidine triphosphate
4-hydroxy-nonenal-glutathione
1-methylhistidine
pseudouridine
2-stearoyl-GPE (18:0)*
1-myristoleoylglycerol (14:1)
3-hydroxymyristate
methylphosphate
spermidine
2-hydroxypalmitate
creatine phosphate
dimethylarginine (SDMA + ADMA)
3-hydroxyoctanoate
cytidine 2′,3′-cyclic monophosphate
beta-hydroxyisovalerate
N-acetylhistidine
myristate (14:0)
linoleoylcarnitine*
guanosine 5′-monophosphate (5′-GMP)
N-alpha-acetylornithine
phenyllactate (PLA)
succinylcarnitine
N-acetylglucosaminylasparagine
isobutyrylcarnitine
malate
17-methylstearate
methionine sulfoxide
palmitoyl sphingomyelin (d18:1/16:0)
cytidine 5′-monophosphate (5′-CMP)
stearoyl sphingomyelin (d18:l/18:0)
N-formylmethionine
1-(1-enyl-oleoyl)-GPE (P-18:1)*
dihydroxyacetone phosphate (DHAP)
glutamate, gamma-methyl ester
prolylglycine
N-acetylglycine
glycerate
1-dihomo-linoleoylglycerol (20:2)
tricosanoyl sphingomyelin (d18:1/23:0)*
2-arachidonoylglycerol (20:4)
3-ketosphinganine
sphingomyelin (d18:1/21:0, d17:1/22:0, d16:1/23:0)*
benzoylcarnitine*
betaine
fructose-6-phosphate
xanthosine
choline phosphate
urate
isoleucylglycine
2-oleoylglycerol (18:1)
N-acetylarginine
sphingomyelin (d18:2/16:0, d18:1/16:1)*
valylglutamine
lysine
1-stearoyl-GPS (18:0)*
glycylisoleucine
1-(1-enyl-palmitoyl)-2-palmitoyl-GPC (P-16:0/16:0)*
1-palmitoyl-2-oleoyl-GPC (16:0/18:1)
uridine 5′-diphosphate (UDP)
phosphate
stearidonate (18:4n3)
6-phosphogluconate
7-hydroxycholesterol (alpha or beta)
leucylglycine
5-methyluridine (ribothymidine)
adenosine2′-monophosphate (2-AMP)
1-stearoyl-2-oleoyl-GPE (18:0/18:1)
tiglylcarnitine
ribitol
cytidine 5-monophospho-N-acetylneuraminic acid
N6,N6,N6-trimethyllysine
valylleucine
S-adenosylhomocysteine (SAH)
trans-4-hydroxyproline
beta-guanidinopropanoate
uridine 5′-triphosphate (UTP)
1-(1-enyl-palmitoyl)-2-linoleoyl-GPC (P-16:0/18:2)*
sphingomyelin (d18:1/20:0, d16:1/22:0)*
2-linoleoylglycerol (18:2)
1-(1-enyl-stearoyl)-2-arachidonoyl-GPE (P-18:0/20:4)*
5-aminovalerate
pyruvate
N6-carboxymethyllysine
2-palmitoleoylglycerol (16:1)*
2′-deoxyguanosine
N-acetylasparagine
laurylcarnitine
1-(1-enyl-palmitoyl)-2-linoleoyl-GPE (P-16:0/18:2)*
N-acetylglutamine
cytidine 5′-diphosphocholine
phenylalanylglycine
N-acetylvaline
5-hydroxyindoleacetate
glycerophosphoinositol*
1,2-dipalmitoleoyl-GPC (16:1/16:1)*
1-(1-enyl-palmitoyl)-2-arachidonoyl-GPE (P-16:0/20:4)*
leucylglutamine*
N6-carbamoylthreonyladenosine
3-hydroxyisobutyrate
histidine
1,2-dipalmitoyl-GPC (16:0/16:0)
3-methylhistidine
2-aminoadipate
lactosyl-N-palmitoyl-sphingosine
pantothenate
threonine
thymidine
adenosine 3-monophosphate (3′-AMP)
5-oxoproline
threonate
2-palmitoylglycerol (16:0)
1-oleoyl-2-linoleoyl-GPC (18:1/18:2)*
valylglycine
1-(1-enyl-palmitoyl)-GPE (P-16:0)*
N-monomethylarginine
arachidonate (20:4n6)
1-docosahexaenoylglycerol (22:6)
butyrylcarnitine
ethylmalonate
dihomo-linolenate (20:3n3 or n6)
1-linoleoyl-2-arachidonoyl-GPC (18:2/20:4n6)*
lactate
ribose
betaine aldehyde
homoserine lactone
acetyl CoA
sphingomyelin (d18:1/24:1, d18:2/24:0)*
S-(3-hydroxypropyl)mercapturic acid (HPMA)
5-methylcytidine
1,2-distearoyl-GPC (18:0/18:0)
phenol sulfate
behenoyl sphingomyelin (d18:1/22:0)*
prostaglandin E2
tyrosine
1,2-dilinoleoyl-GPC (18:2/18:2)
stearoyl ethanolamide
1-palmitoyl-3-linoleoyl-glycerol (16:0/18:2)*
glutarate (pentanedioate)
indolelactate
malonylcarnitine
S-lactoylglutathione
sulfate*
15-methylpalmitate
O-sulfo-L-tyrosine
phenylacetylglycine
N-delta-acetylornithine
alpha-tocopherol
2′-deoxyuridine
adenosine
glycerol 3-phosphate
pentadecanoate (15:0)
sphingomyelin (d18:2/23:0, d18:1/23:1, d17:1/24:1)*
1-(1-enyl-palmitoyl)-2-arachidonoyl-GPC (P-16:0/20:4)*
eicosenoate (20:1)
glycylvaline
palmitoylcholine
phenol red
ribonate
palmitate (16:0)
5-hydroxylysine
stachydrine
2-methylbutyrylglycine
methyl glucopyranoside (alpha + beta)
S-methylglutathione
1-stearoyl-GPG (18:0)
inosine
uridine 3′-monophosphate (3′-UMP)
sarcosine (N-Methylglycine)
1-palmitoyl-2-linolenoyl-GPC (16:0/18:3)*
adenosine 3′,5′-cyclic monophosphate (cAMP)
dimethylglycine
alanine
isoleucine
cysteine sulfinic acid
leucine
N-acetylphenylalanine
nervonate (24:1n9)*
citrate
glucose
2-hydroxystearate
adenine
valine
sphingomyelin (d18:1/14:0, d16:1/16:0)*
margarate (17:0)
allantoin
alanylleucine
p-cresol sulfate
N-acetylalanine
1-margaroylglycerol (17:0)
nicotinamide riboside
1-myristoylglycerol (14:0)
1-palmitoyl-2-palmitoleoyl-GPC (16:0/16:1)*
N-methylproline
eicosapentaenoate (EPA; 20:5n3)
taurine
1-pentadecanoylglycerol (15:0)
phosphopantetheine
2-docosahexaenoylglycerol (22:6)*
tryptophylglycine
5-methyltetrahydrofolate (5MeTHF)
1-stearoyl-2-arachidonoyl-GPS (18:0/20:4)
thymidine 5′-monophosphate
phenylalanine
erythritol
UDP-glucuronate
sphingomyelin (d18:2/14:0, d18:1/14:1)*
mannitol/sorbitol
1-stearoyl-2-linoleoyl-GPE (18:0/18:2)*
succinate
fumarate
adenylosuccinate
prolylglutamine
sphingomyelin (d18:2/24:1, d18:1/24:2)*
S-1-pyrroline-5′-carboxylate
pipecolate
1-palmitoyl-2-linoleoyl-GPC (16:0/18:2)
10-nonadecenoate (19:1n9)
uridine 5-monophosphate (UMP)
alpha-hydroxyisovalerate
1-lignoceroyl-GPC (24:0)
sphingomyelin (d18:1/20:1, d18:2/20:0)*
methionine
cholesterol
O-methyltyrosine
2-myristoylglycerol (14:0)
kynurenine
5-methyl-2′-deoxycytidine
putrescine
homocitrulline
5-aminolevulinate
oleate/vaccenate (18:1)
flavin adenine dinucleotide (FAD)
N-palmitoyl-sphinganine (d18:0/16:0)
tyrosylglycine
glucose 6-phosphate
N-stearoyltaurine
glycylleucine
alpha-ketoglutarate
sphingomyelin (d18:1/17:0, d17:1/18:0, d19:1/16:0)
1-(1-enyl-stearoyl)-2-oleoyl-GPE (P-18:0/18:1)
glutathione, oxidized (GSSG)
sphingomyelin (d18:1/15:0, d16:1/17:0)*
N-acetylisoleucine
3-aminoisobutyrate
1-oleoyl-GPE (18:1)
6-oxopiperidine-2-carboxylic acid
palmitoyl ethanolamide
methylmalonate (MMA)
cytosine
2′-deoxyinosine
N-acetylglucosamine 6-phosphate
docosapentaenoate (n3 DPA; 22:5n3)
dihomo-linoleate (20:2n6)
1-stearoyl-2-arachidonoyl-GPC (18:0/20:4)
adenosine 5-diphosphoribose (ADP-ribose)
histidine methyl ester
1-(1-enyl-palmitoyl)-2-oleoyl-GPC (P-16:0/18:1)*
erucate (22:1n9)
1-stearoyl-2-arachidonoyl-GPE (18:0/20:4)
N6,N6-dimethyladenosine
linolenate [alpha or gamma; (18:3n3 or 6)]
maleate
3-(4-hydroxyphenyl)lactate
glutamine
adrenate (22:4n6)
N-acetylcysteine
nicotinamide adenine dinucleotide (NAD+)
7-methylguanosine
N-palmitoyl-sphingosine (d18:1/16:0)
gamma-glutamyl-epsilon-lysine
1-stearoyl-2-linoleoyl-GPC (18:0/18:2)*
palmitoleate (16:1n7)
N6-carboxyethyllysine
cysteine
trimethylamine N-oxide
sphingomyelin (d18:1/22:1, d18:2/22:0, d16:1/24:1)*
1-palmitoyl-2-oleoyl-GPS (16:0/18:1)
1-linoleoyl-GPE (18:2)*
citrulline
10-heptadecenoate (17:1n7)
trigonelline (N′-methylnicotinate)
quinolinate
oleoyl ethanolamide
N2,N2-dimethylguanosine
docosahexaenoate (DHA; 22:6n3)
hippurate
adenosine 5-diphosphate (ADP)
1-palmitoyl-2-stearoyl-GPC (16:0/18:0)
1-(1-enyl-palmitoyl)-2-palmitoleoyl-GPC (P-16:0/16:1)*
1-palmitoyl-GPE (16:0)
uridine
1-palmitoleoylglycerol (16:1)*
1-palmitoleoyl-2-oleoyl-GPC (16:1/18:1)*
N2-acetyllysine/N6-acetyllysine
AICA ribonucleotide
2′-deoxyadenosine 5-monophosphate
penicillin G
linoleate (18:2n6)
1-palmitoleoyl-3-oleoyl-glycerol (16:1/18:1)*
N-acetylneuraminate
galactonate
adenosine 5′-monophosphate (AMP)
methionine sulfone
guanosine 5′-diphospho-fucose
1-palmitoyl-2-oleoyl-GPE (16:0/18:1)
riboflavin (Vitamin B2)
N-acetyl-aspartyl-glutamate (NAAG)
inositol 1-phosphate (I1P)
docosadienoate (22:2n6)
1-palmitoylglycerol (16:0)
mead acid (20:3n9)
argininosuccinate
5-methylthioadenosine (MTA)

Likewise, targeted metabolomics profiling revealed an accumulation of the TCA cycle intermediates citrate, α-KG, succinate, and malate in Pkd1^{-/-} cells as compared to controls (FIGS. 8C and D). These results show that the response to Pkd1 loss is strikingly similar between Pdk1-mutant MEFs and kidneys, supporting the validity of our cell model for further mechanistic investigations.

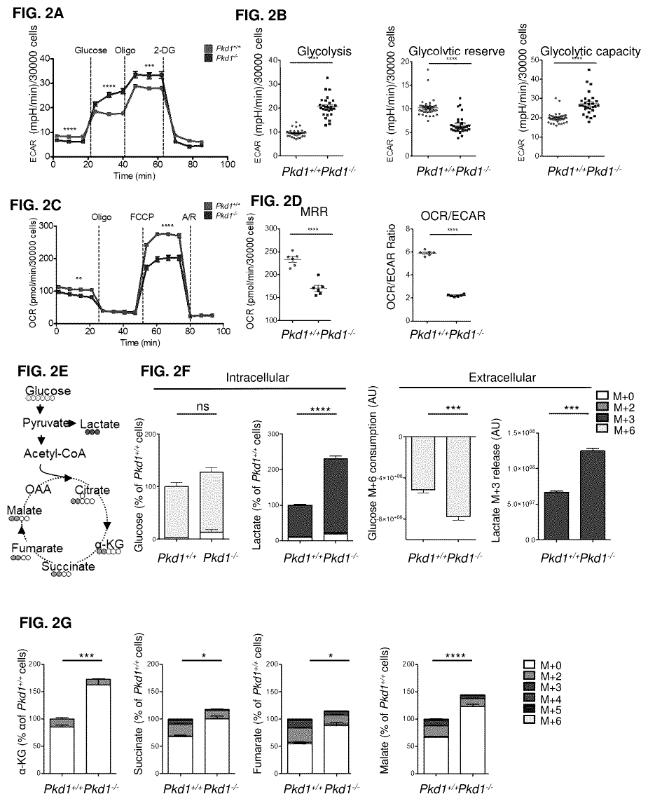

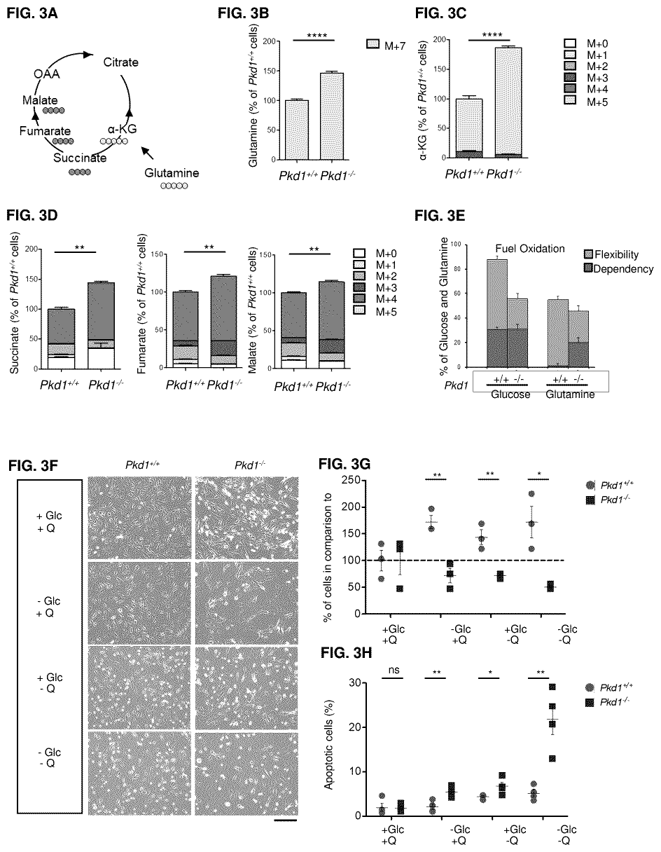

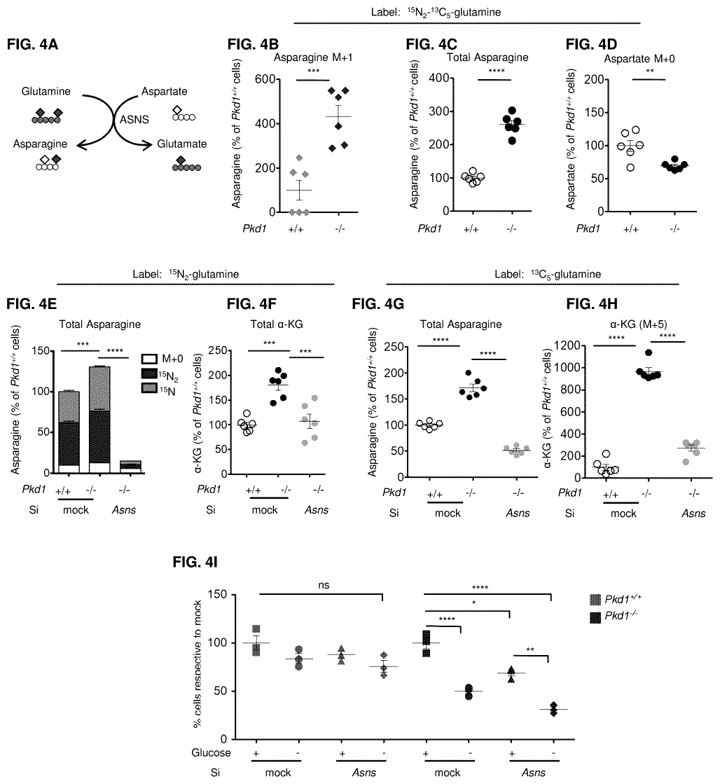

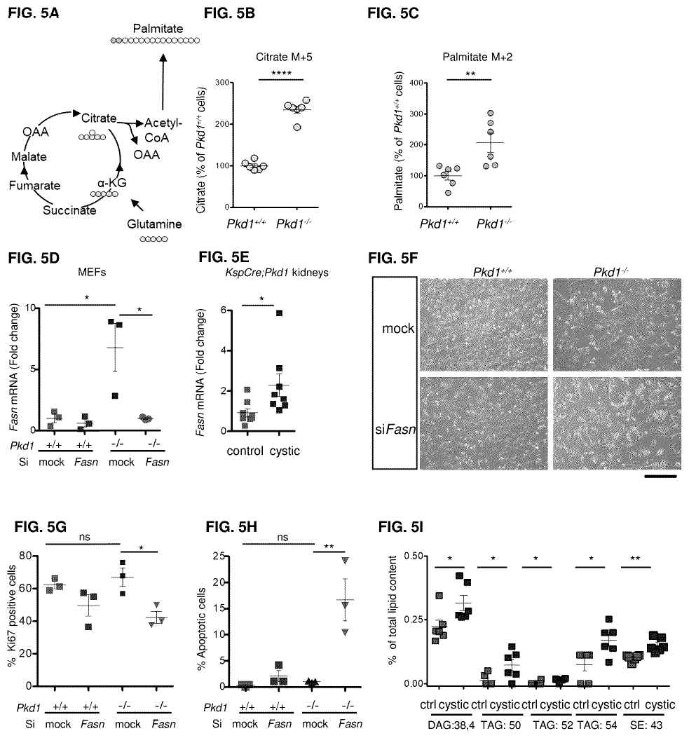

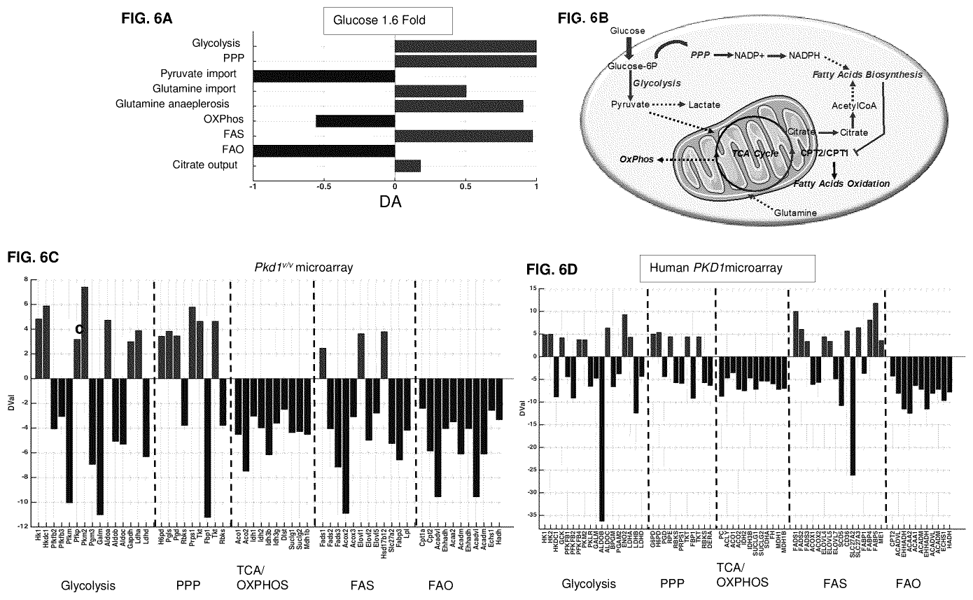

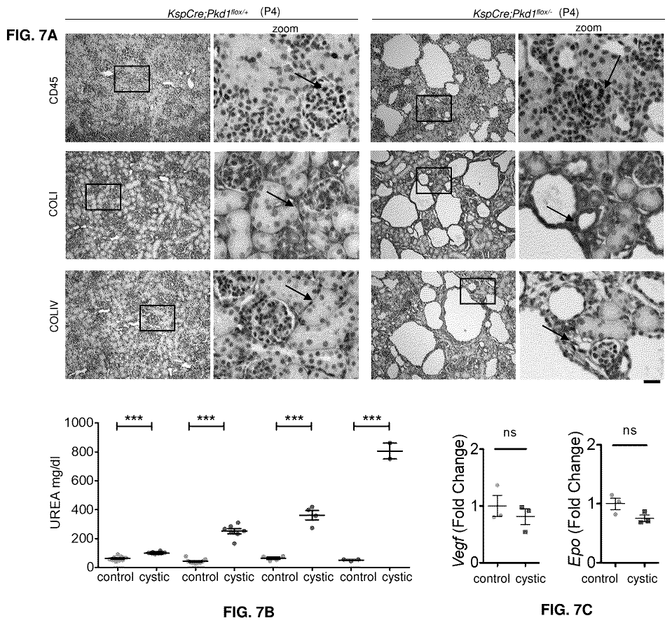

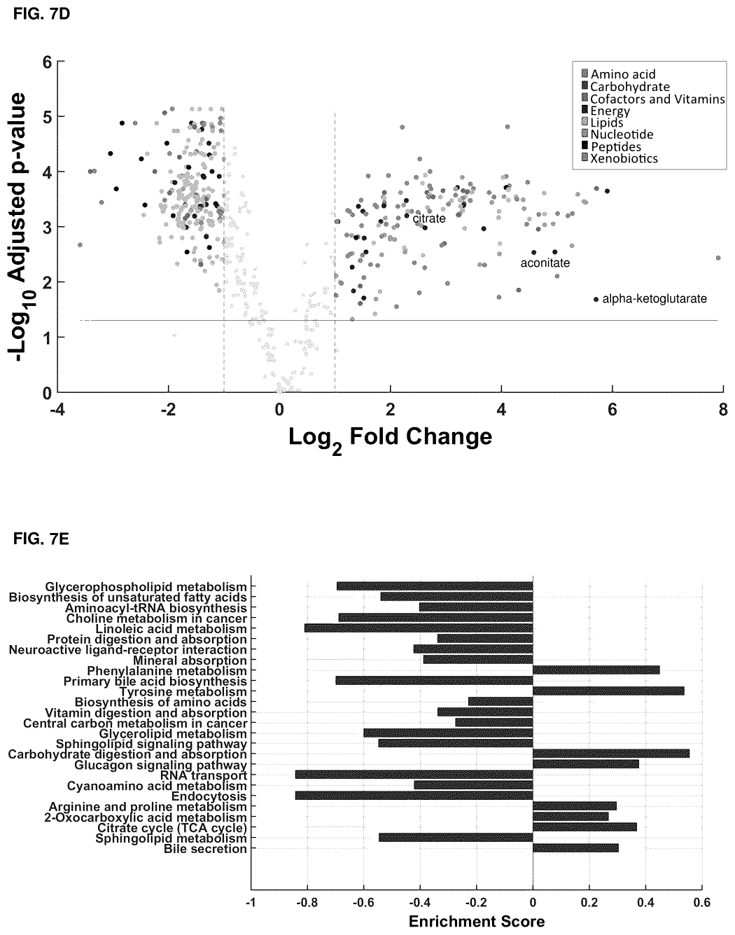

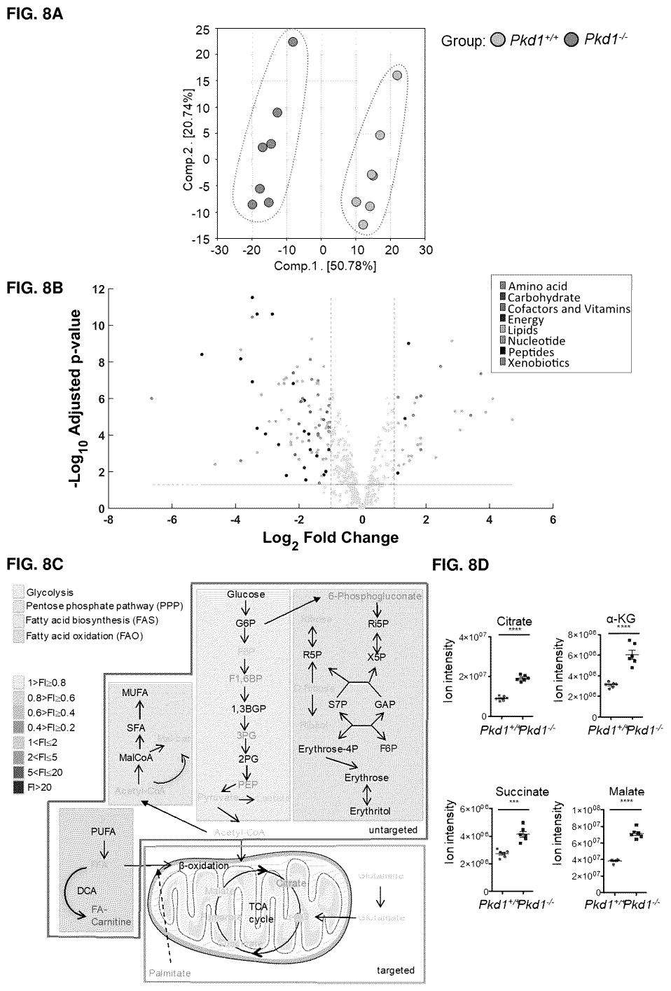

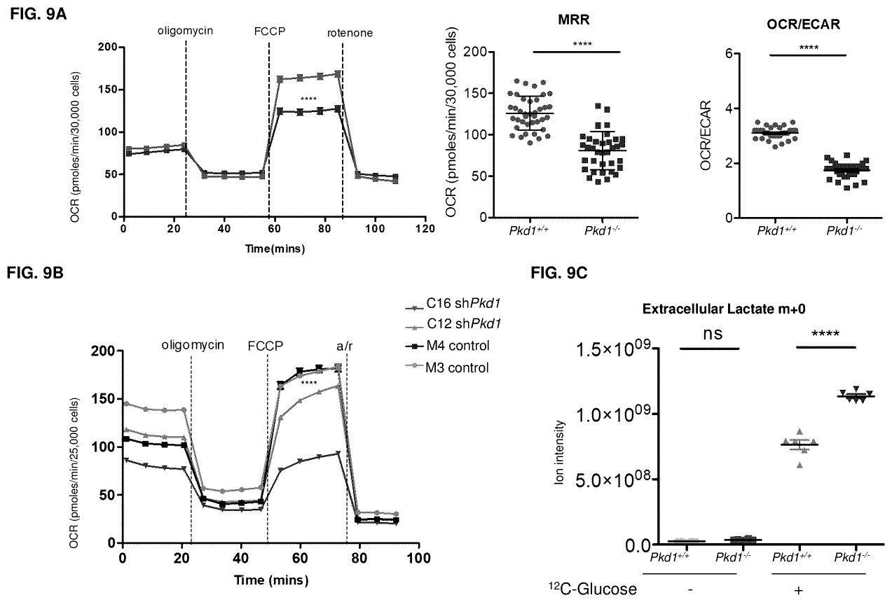

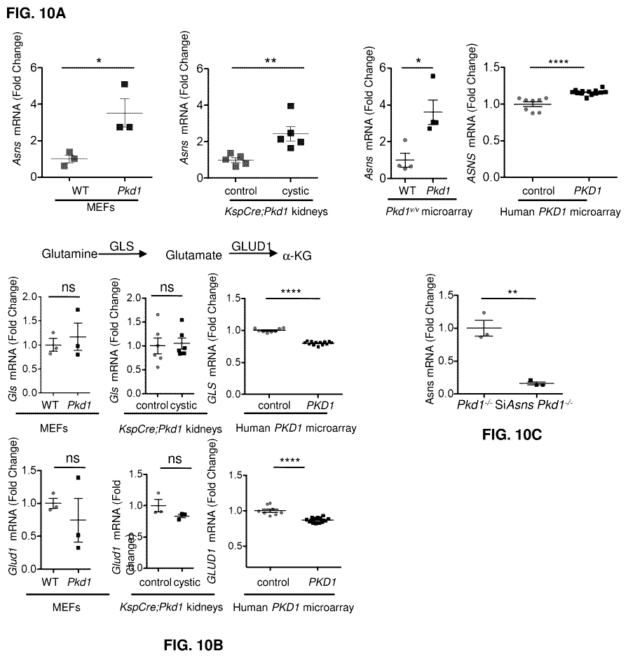

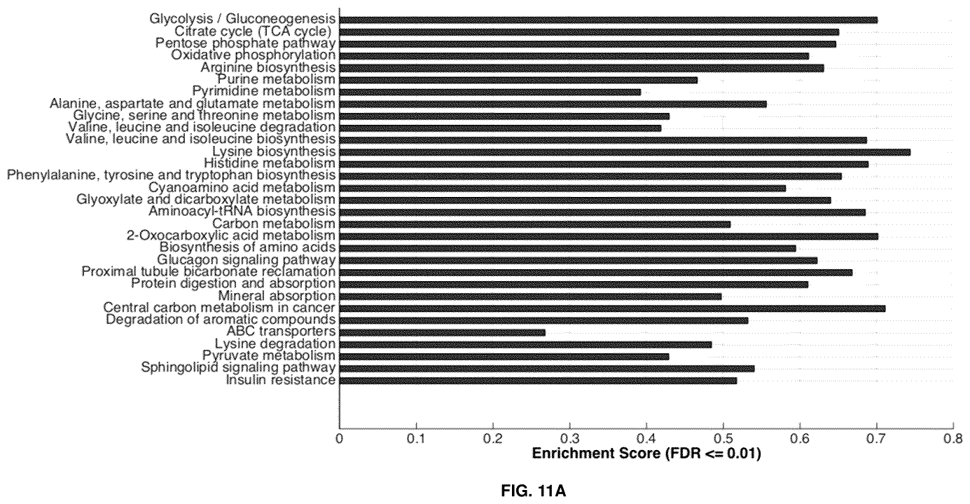

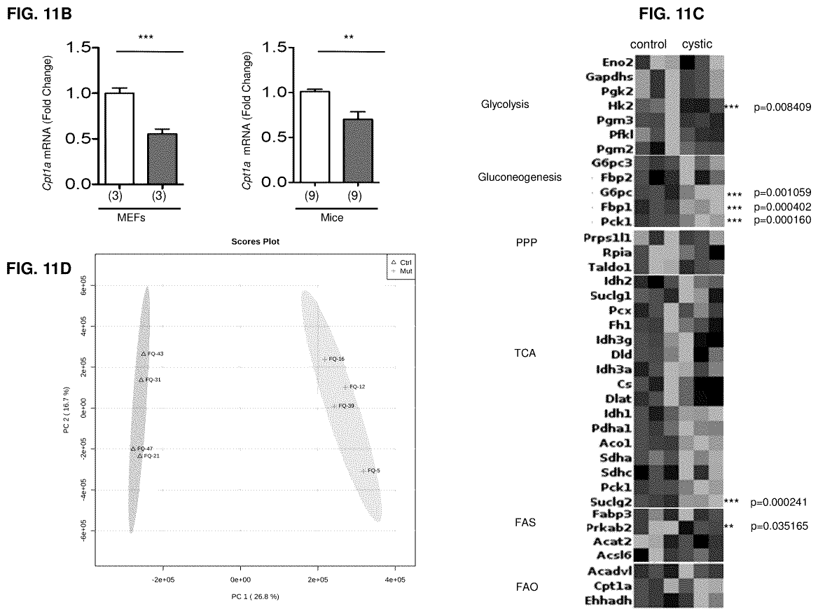

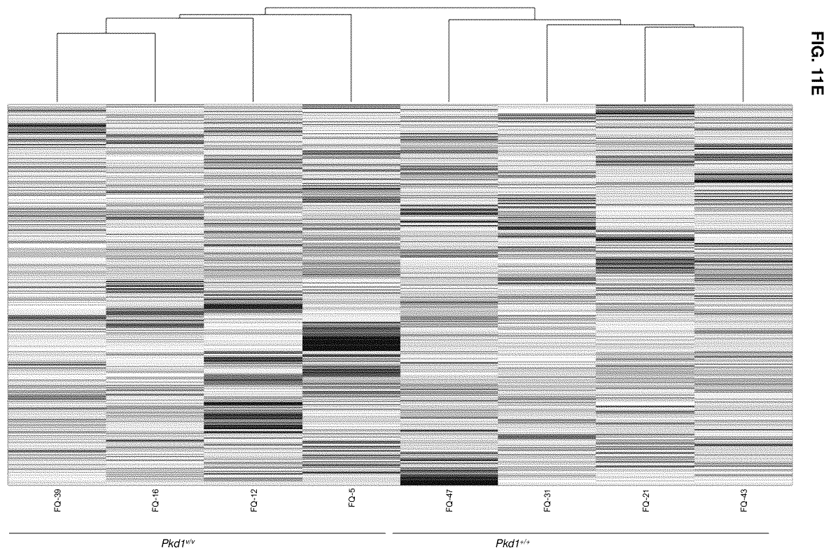

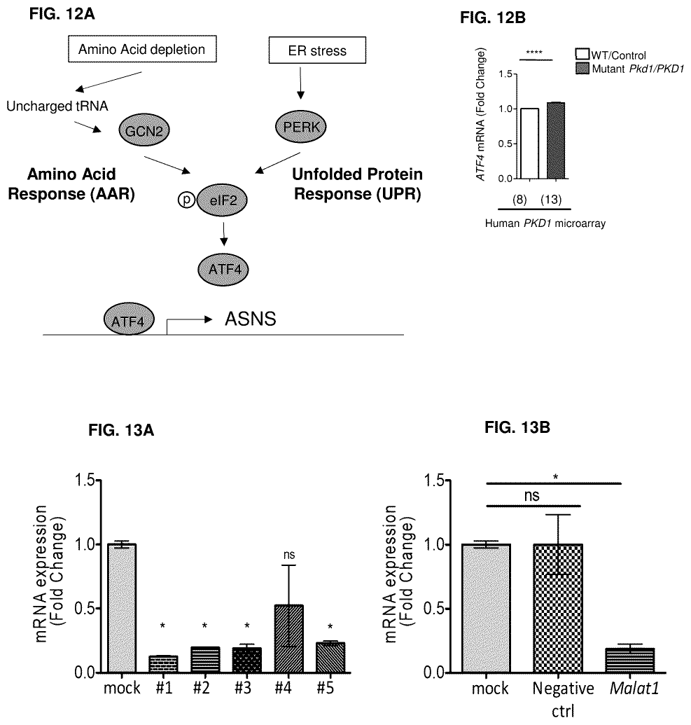

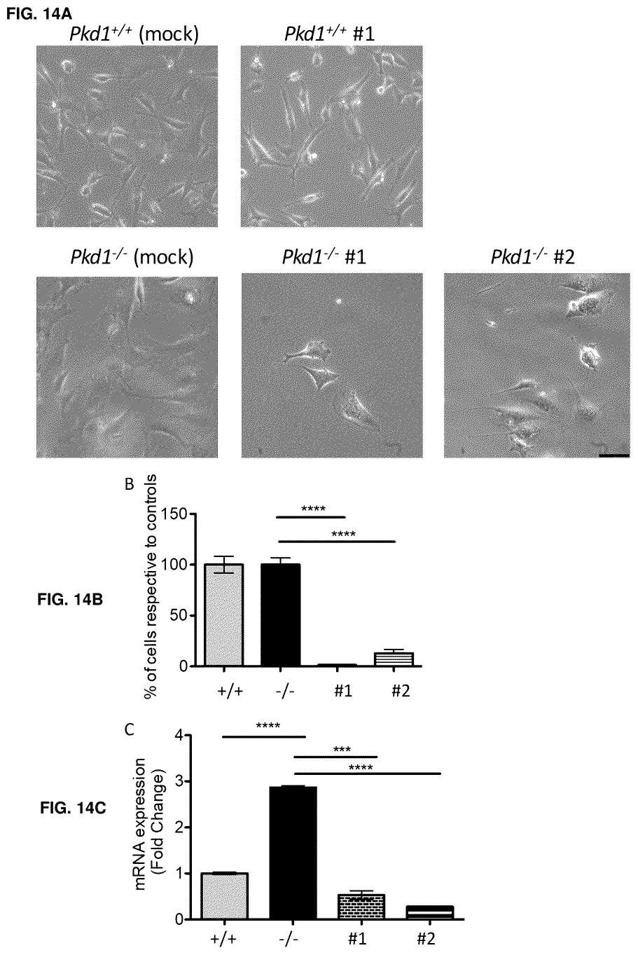

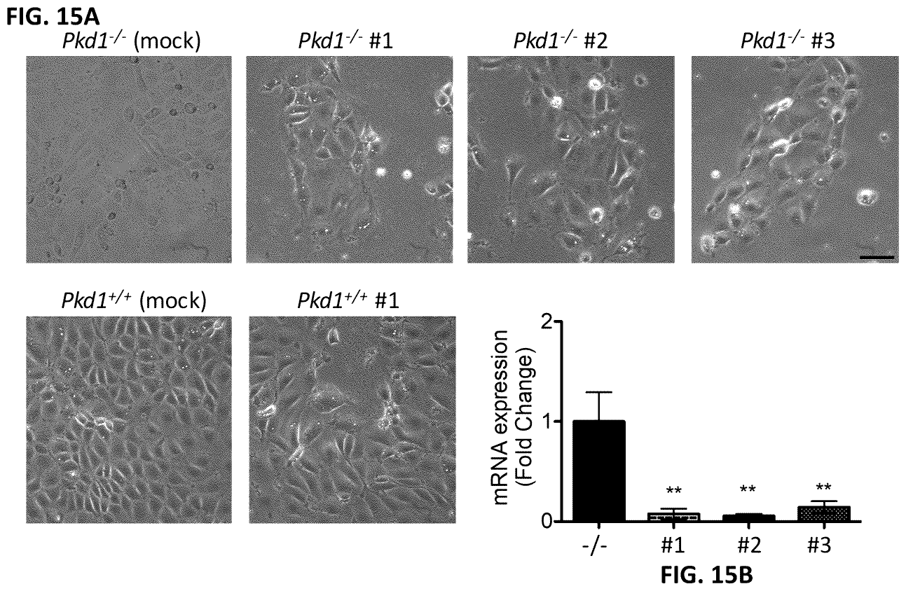

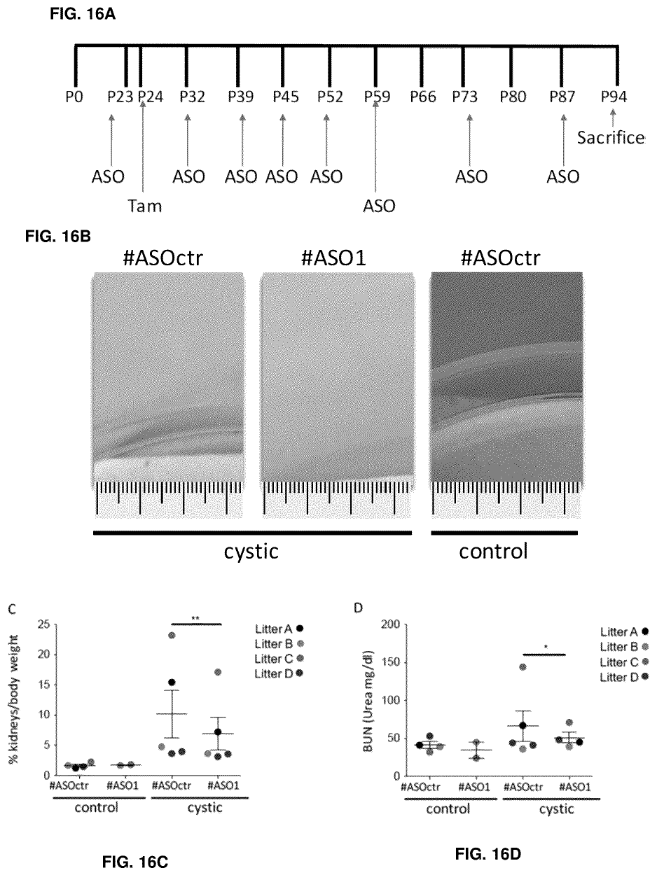

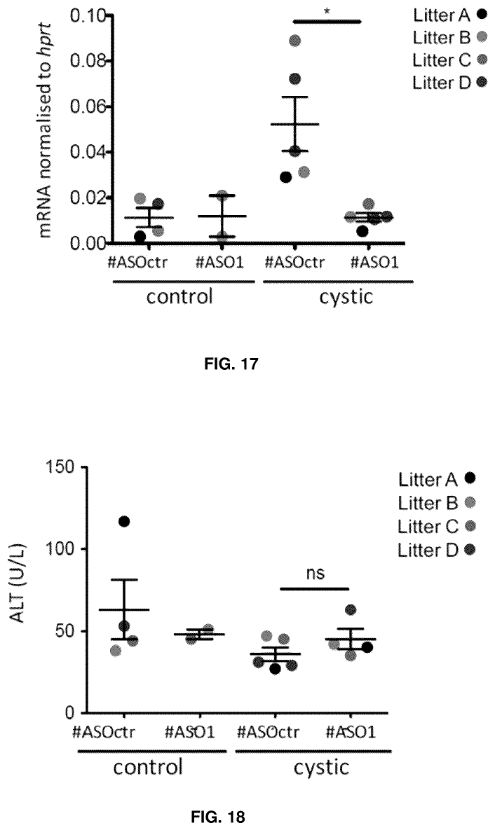

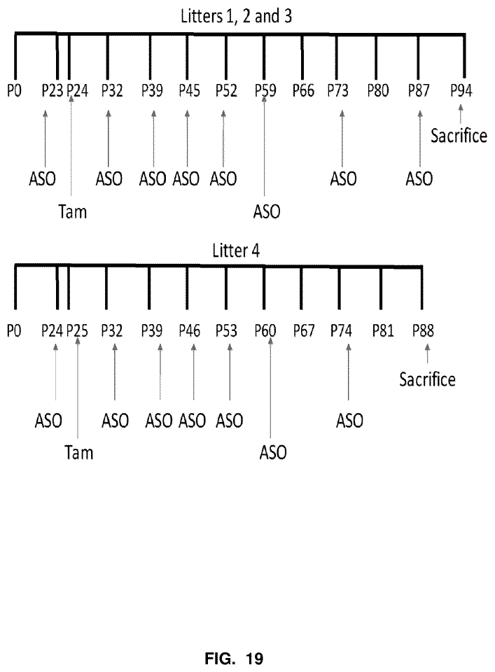

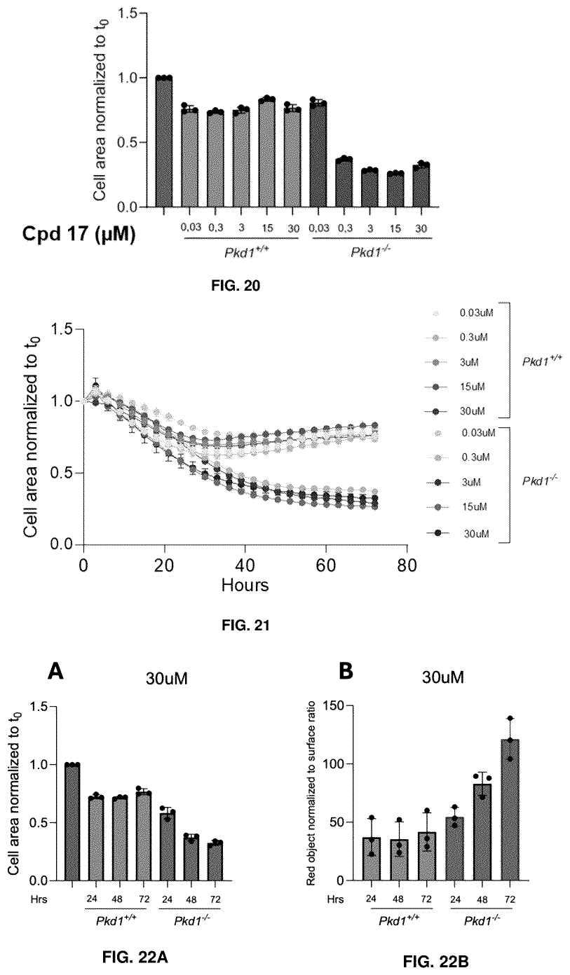

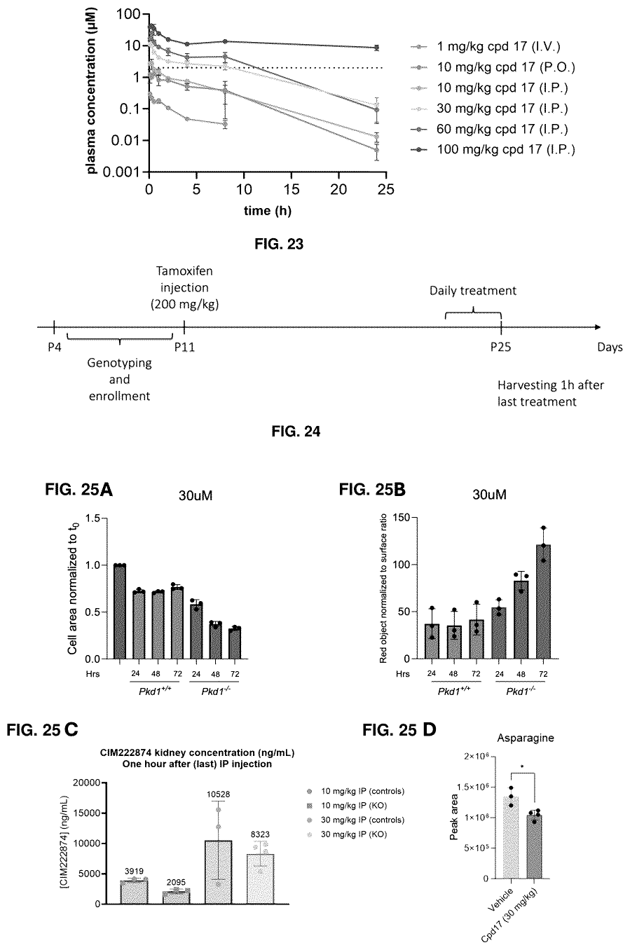

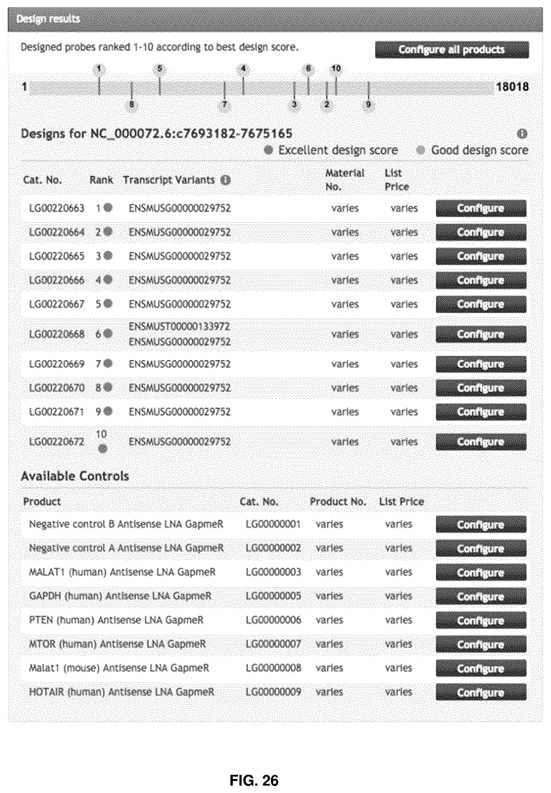

The inventors first assessed whether the TCA cycle alterations could be associated with a defective cellular respiration in Pkd1^{-/-} cells as recently reported^{8,15}. Consistent with this hypothesis, extracellular flux analysis of Pkd1^{-/-} MEFs showed enhanced glycolysis (FIGS. 2A and B) and defective respiration (Oxygen Consumption Rate, OCR) (FIGS. 2C and D) as compared to Pkd1^{+/+}. Similar results were generated on OCR were generated using primary Pkd1^{-/-} MEFs (FIG. 9A) and murine Inner Medullary Collecting Duct Cells (mIMCD cells) where the Pkd1 gene was silenced (FIG. 9B)⁴⁵.

### Example 2

### Glucose Entry in the TCA Cycle is Reduced in Pkd1^{-/-} Cells

The inventors then further investigate to what extent the loss of Pkd1 affects central carbon metabolism. To track the contribution of glucose, one of the major carbon sources for the cells, to the TCA cycle in the Pkd1^{-/-} cells, the inventors incubated cells with uniformly labelled 13C-glucose and followed the incorporation of 13C in downstream metabolites (FIG. 2E). Relative to control cells, Pkd1^{-/-} cells took up more glucose and converted it into lactate, which is released into the culture medium (FIG. 2F and FIG. 9C). The results showed that lactate almost entirely derives from glucose (FIG. 2F and FIG. 9C). The data also showed that the glucose-derived isotopologues of α-KG (M+2), succinate (M+2), fumarate (M+2), and malate (M+2) were all decreased in Pkd1^{-/-} MEFs, indicating a reduced contribution of glucose to the TCA cycle (FIG. 2G).

### Example 3

### Rewiring of Glutamine Metabolism in Pkd1^{-/-} Cells

The inventors next assessed the utilisation of glutamine, another key carbon source for the cells. To this end, the inventors incubated cells with ¹³C₅-¹⁵N₂-glutamine and the fate of glutamine-derived carbons and nitrogen was assessed by LC-MS (FIG. 3A). Pkd1^{-/-} cells exhibited increased glutamine uptake, compared to the controls (FIG. 3B), and glutamine-derived (M+5) α-KG (FIG. 3C). In addition, Pkd1^{-/-} cells diverted glutamine towards the TCA cycle, as demonstrated by the increased levels of glutamine-derived succinate (M+4), fumarate (M+4), and malate (M+4) in Pkd1^{-/-} as compared with Pkd1^{+/+} cells (FIG. 3D). Next, the inventors measured the dependency and flexibility of Pkd1^{-/-} cells relative to their controls (using the XF Mito Fuel Flex test, see methods). This assay measures the OCR of Pkd1^{+/+} and Pkd1^{-/-} cells in the presence of glucose and glutamine followed by their blockade using specific inhibitors of the two metabolic pathways (2-Deoxy-D-glucose and BPTES, respectively). Data showed that Pkd1^{-/-} cells are dependent on glutamine for their OCR production, while the glucose-driven OCR is overall reduced (FIG. 3E). These data suggest that cells lacking Pkd1 increased their utilisation of glutamine to fuel the TCA cycle and maintain their OCR, most likely as a compensatory mechanism due to the reduced funneling of glucose into mitochondria. Based on these findings, the inventors hypothesised that cells lacking functional Pkd1 would become addicted to glutamine in addition to glucose. Indeed, starvation from either glutamine or glucose reduced cell numbers (FIGS. 3F and G) and increased cell death in Pkd1^{-/-} cells (FIG. 3H) relative to the controls. Importantly, starvation from both carbon sources had a synergistic effect on apoptosis (FIG. 3H).

Following the fate of the ¹⁵N₂-labelled glutamine the inventors noticed that there is a significant increase of 15N-asparagine in the Pkd1^{-/-} cells (FIG. 4B) and a concomitant increase of the overall levels of labelled asparagine derived from glutamine (FIG. 4C), while aspartate was decreased in the same cells (FIG. 4D). The inventors therefore hypothesised that Pkd1^{-/-} cells exhibit increase asparagine synthesis from glutamine. Further, quantitative RT-PCR revealed that Asparagine synthase, the enzyme that generates asparagine from aspartate (FIG. 4A), is significantly upregulated in cells and murine kidneys (FIG. 10A). Furthermore, analysis of microarrays from murine and human samples (see below) confirmed a significant upregulation of this enzyme in both systems (FIG. 10A). Of interest, quantitative RT-PCR revealed no difference in the expression levels of the two enzymes Gls and Glud1 were not (FIG. 10B). To evaluate the relevance of asparagine biosynthesis, the inventors silenced Asns in both Pkd1^{+/+} and in Pkd1^{-/-} cells (FIGS. 4E and 4G and FIG. 10C). The silencing of Asns reduced the intracellular levels of total asparagine in Pkd1^{-/-} cells (FIGS. 4E and 4G). Metabolic tracing with ¹⁵N₂-glutamine showed that silencing of Asns indeed reduced the total levels of α-KG (FIG. 4F). Furthermore, metabolic tracing with ¹³C₅-glutamine showed a significant reduction in the glutamine-derived α-KG (M+5) (FIG. 4H) showing that Asns plays a central role in glutamine fueling of the TCA cycle in Pkd1^{-/-} cells (FIGS. 4F and 4H). Importantly, the down-regulation of Asns also resulted in reduced cell numbers in Pkd1^{-/-} cells, but not in controls (FIG. 4I). Furthermore, when siAsns:Pkd1^{-/-} cells were subject to glucose starvation the inventors noticed a further decrease in cell numbers compared to controls treated in the same conditions (FIG. 4I). The inventors conclude that Asns is essential for Pkd1^{-/-} cells survival when these are glucose deprived. Thus, the present data show that increased glutaminolysis, interlinked with asparagine metabolism is an important feature of PKD and targeting Asns alone or in conjunction with glycolysis offer a novel therapeutic opportunity.

### Example 4

### Pkd1 Loss Leads to Increased De Novo Fatty Acid Biosynthesis

Of interest, the experiments of glutamine tracing also revealed that glutamine is used in an anaplerotic manner by Pkd1^{-/-} cells. Indeed, the increased level of citrate (M+5) in the ¹³C₅-¹⁵N₂-Glutamine labelling experiment (FIGS. 5A and B) suggested that glutamine undergoes reductive carboxylation in Pkd1^{-/-} cells. Given that reductive carboxylation has been linked with synthesis of lipogenic acetyl-CoA, the inventors examined the labelling of palmitate, a fatty acid generated via de novo fatty acid biosynthesis. Consistent with this hypothesis, the inventors noticed an increased labelling of glutamine-derived palmitate (M+2) (FIG. 5C) in Pkd1^{-/-} cells compared to controls, although higher isotopologues could not be detected in this assay. Next, the inventors tested whether Pkd1^{-/-} cells showed increased expression of fatty acids synthase (Fasn), a key enzyme involved in FAS. The inventors found that Fasn is highly expressed in Pkd1^{-/-} cells and kidneys of KspCre;Pkd1^{flox/-} animals (FIGS. 5D and E). Furthermore, its silencing reduced cell proliferation and enhanced cell apoptosis in Pkd1^{-/-} cells (FIGS. 5F, G and H). In further support of an alteration in lipid metabolism, lipidomics profiling revealed a significant increase in diacylglycerols (DAG), triacyglycerols (TAG), and sterol esters (SE) in cystic kidneys compared to controls (FIG. 5I).

Overall, enhanced de novo fatty acids biosynthesis is a feature of PKD observed both in cells and in murine tissues and this process is necessary for Pkd1^{-/-} cells to proliferate and survive.

Treatment of a murine model of PKD with an inhibitor of FAS slow down the progression of cystogenesis. Therefore de novo fatty acid biosynthesis inhibition offers a good therapeutic strategy in ADPKD.

### Example 5

### Mathematical Modelling and Global Analysis of Transcriptional Profiling Reveals Coordinated Changes in Bioenergetic Pathways in PKD

To gather a broader understanding of the metabolic changes observed, and to predict whether the different pathways are causally linked, the inventors performed an in silico study. To this end the inventors applied a recently described algorithm to predict changes in metabolic fluxes and metabolites across two conditions in a genome-scale metabolic model including 785 metabolites linked by 2589 enzymatic reactions (Differential Flux Balance Analysis, DFA)⁴⁰. After removing all liver-specific functions, the inventors used DFA to simulate in silico an increase in glucose uptake by imposing a constrain of enhanced glucose input driven by two transporters (TCDB: 2.A.21.3.6 and TCDB: 2.A.1.1.29, see methods). To this end, the inventors used as input the amount of increase in glucose uptake (1.6 folds) observed in the tracing experiments of Pkd1^{-/-} MEFs as compared to Pkd1^{+/+} controls (FIG. 2F). Next, the inventors analyzed the complete list of metabolites ranked according to their predicted change. This list was used as the input to KEGG-Pathways Based Enrichment Analysis, resulting in 31 pathways significantly enriched for altered metabolites (FDR≤0.01). The most significant were glycolysis, TCA cycle, PPP, OXPHOS and FAS (FIG. 11A). Importantly, this method allows to assign a direction to all the metabolic fluxes and to further predict additional alterations. Besides the metabolic pathways above the inventors noticed a remarkable increase in glutamine uptake in the model system (FIGS. 6A and B and Table III), consistent with the glutamine labelling experiments.

TABLE III
Subset of the in silico model reactions used to evaluate the change of metabolic pathways depicted
in FIGS. 6A and 6B. Column 1: reactions; column 2: enzymes and transporters.

| Reaction | Enzyme/Transporter |
|---|---|
| Glycolysis/Gluconeogenesis | |
| Glucose(s) + Na+(s) -> Glucose(c) + Na+(c) | TCDB:2.A.21.3.6 |
| Glucose(s) <=> Glucose(c) | TCDB:2.A.1.1.29 |
| ATP(c) + Glucose(c) -> ADP(c) + Glucose-6P(c) | EC:2.7.1.1 |
| Glucose-6P(c) <=> Fructose-6P(c) | EC:5.3.1.9 |
| H2O(c) + Fructose-1,6PP(c) -> Pi(c) + Fructose-6P(c) | EC:3.1.3.11 |
| Fructose-1,6PP(c) <=> DHAP(c) + GAP(c) | EC:4.1.2.13 |
| GAP(c) <=> DHAP(c) | EC:5.3.1.1 |
| NAD+(c) + Pi(c) + GAP(c) <=> NADH(c) + 1,3DPG(c) | EC:1.2.1.12 |
| ATP(c) + 3PG(c) <=> ADP(c) + 1,3DPG(c) | EC:2.7.2.3 |
| ATP(c) + Pyruvate(c) <=> ADP(c) + PEP(c) | EC:2.7.1.40 |
| NAD+(c) + L-Lactate(c) <=> NADH(c) + Pyruvate(c) | EC:1.1.1.27 |
| ATP(c) + Fructose-6P(c) -> ADP(c) + Fructose-1,6PP(c) | EC:2.7.1.11 |
| Pentose phospate pathways to fatty acid | |
| H2O(c) + Glucono-1,5-lactone-6P(c) <=> 6-Phospho-D-gluconate(c) | EC:3.1.1.31 |
| NADP+(c) + 6-Phospho-D-gluconate(c) <=> NADPH(c) + CO2(c) + Ribulose-5P(c) | EC:1.1.1.44 |
| Glucose-6P(c) <=> Fructose-6P(c) | EC:5.3.1.9 |
| ATP(c) + Ribose-5P(c) -> AMP(c) + PRPP(c) | EC:2.7.6.1 |
| ATP(c) + Fructose-6P(c) -> ADP(c) + Fructose-1,6PP(c) | EC:2.7.1.11 |
| Pyruvate and glutamine cytosol/mitochondria exchange | |
| Pyruvate(c) + H+(PG)(c) -> Pyruvate(m) + H+(PG)(m) | Active_transport |
| Glutamine(m) + H+(PG)(c) -> Glutamine(c) + H+(PG)(m) | Transport_reaction |
| Glutamine(c) + H+(PG)(c) -> Glutamine(m) + H+(PG)(m) | Facilitated_diffusion |
| Glutamine(c) <=> Glutamine(m) | Utilized_transport |
| Glutamine Anaplerosis | |
| NAD+(m) + Isocitrate(m) <=> NADH(m) + Oxalosuccinate(m) | EC:1.1.1.42 |
| NADP+(m) + Isocitrate(m) <=> NADPH(m) + Oxalosuccinate(m) | EC:1.1.1.42 |
| Oxalosuccinate(m) <=> CO2(m) + AKG(m) | EC:1.1.1.42 |
| Citrate(m) <=> Isocitrate(m) | EC:4.2.1.3 |
| NAD+(m) + CoA(m) + AKG(m) <=> NADH(m) + CO2(m) + Succinyl-CoA(m) | EC:1.2.4.2 |
| Oxidative phosphorylation | |
| NAD+(m) + Malate(m) <=> NADH(m) + OAA(m) | EC:1.1.1.37 |
| Malate(m) <=> H2O(m) + Fumarate(m) | EC:4.2.1.2 |
| Succinate(m) + Ubiquinone(m) -> Fumarate(m) + Ubiquinol(m) | EC:1.3.5.1 |
| CoA(m) + Succinate(m) + GTP(m) <=> Pi(m) + GDP(m) + Succinyl-CoA(m) | EC:6.2.1.4 |
| Succinate(m) + Ubiquinone(m) -> Fumarate(m) + Ubiquinol(m) | EC:1.3.5.1 |
| NAD+(m) + 4 H+(PG)(c) + Ubiquinol(m) <=> NADH(m) + 4 H+(PG)(m) + | EC:1.6.5.3 |
| Ubiquinone(m) | |
| 4 H+(PG)(m) + 2 Ferricytochrome_C(m) + Ubiquinol(m) -> 4 H+(PG)(c) + 2 | EC:1.10.2.2 |
| Ferrocytochrome_C(m) + Ubiquinone(m) | |
| O2(m) + 4 H+(PG)(m) + 4 Ferrocytochrome_C(m) -> 2 H2O(m) + 4 H+(PG)(c) + 4 | EC:1.9.3.1 |
| Ferricytochrome_C(m) | |
| H2O(m) + ATP(m) + 3 H+(PG)(m) <=> ADP(m) + Pi(m) + 3 H+(PG)(c) | EC:3.6.3.14 |
| H2O(m) + PPi(m) ->2Pi(m) | EC:3.6.1.1 |
| FAS | |
| ATP(c) + CoA(c) + Citrate(c) -> ADP(c) + Pi(c) + Acetyl-CoA(c) + OAA(c) | EC:2.3.3.8 |
| ATP(c) + Acetyl-CoA(c) + HCO3-(c) -> ADP(c) + Pi(c) + Malonyl-CoA(c) | EC:6.4.1.2 |
| Acetyl-CoA(c) + ACP(c) <=> CoA(c) + Acetyl-ACP(c) | EC:2.3.1.85 |
| Malonyl-CoA(c) + ACP(c) <=> CoA(c) + Malonyl-ACP(c) | EC:2.3.1.85 |
| FAO | |
| ATP(c) + CoA(c) + Arachidonate(c) -> PPi(c) + AMP(c) + Arachidonyl-CoA(c) | EC:6.2.1.3 |
| Palmitoyl-CoA(c) + L-Carnitine(c) <=> CoA(c) + L-Palmitoylcarnitine(c) | EC:2.3.1.21 |
| Palmitoyl-CoA(m) + L-Carnitine(m) <=> CoA(m) + L-Palmitoylcarnitine(m) | EC:2.3.1.21 |
| Citrate(c) <=> Isocitrate(c) | EC:4.2.1.3 |
| Citrate Output | |
| PEP(m) + Citrate(c) <=> PEP(c) + Citrate(m) | TCDB:2.A.29.7.2 |
| AKG(c) + Citrate(m) <=> AKG(m) + Citrate(c) | TCDB:2.A.29.7.2 |
| Succinate(m) + Citrate(c) <=> Succinate(c) + Citrate(m) | TCDB:2.A.29.7.2 |
| H+(PG)(m) + Malate(c) + Citrate(m) <=> H+(PG)(c) + Malate(m) + Citrate(c) | TCDB:2.A.29.7.2 |
| H+(PG)(m) + Citrate(m) + Oxalate(c) <=> H+(PG)(c) + Citrate(c) + Oxalate(m) | TCDB:2.A.29.7.2 |
| Malate(m) + Citrate(c) <=> Malate(c) + Citrate(m) | TCDB:2.A.29.7.2 |

Further to this, the in silico simulations suggested that CPT1 and CPT2 metabolic fluxes might be reduced as a consequence of increased FAS (FIG. 11A). Indeed, qRT-PCR analysis showed that CPT1 was significantly reduced in cells and kidneys lacking functional Pkd1 (FIG. 11B), further validating the predictions originated by the algorithm and in line with previous findings^{15,46}.

The analysis of the in silico fluxes generated by the model revealed that most of the alterations in the discussed metabolic pathways is coordinated and likely causally linked, to the point that a single change in the increased uptake of glucose recapitulates a broad alteration in the other metabolic pathways (FIG. 6B). The inventors next reasoned that if these changes are indeed coordinated, they should occur in a synchronous fashion in the cystic kidneys. To address this point the inventors first used a qPCR-based transcriptional profiling (qPCR arrays Qiagen™) applied to a KspCre;Pkd1^{flox/-} kidneys analyzed at the same time-point in which metabolomics was performed (P4). Even if the qPCR-based arrays provided a rather limited number of targets, data clearly showed a trend of alterations into key enzymes involved in Glycolysis, PPP, FAS and FAO even if only a few displayed significant changes between the Cystic and the control samples (FIG. 11C and Table IV).

### Table IV: Alterations of Key Enzymes

Glucose metabolism: Acly, Aco1, Aco, Ag,I, Aldoa, Aldob, Aldoc, Bpgm, Cs, Dlat, Dld, Dlst, Eno1, Eno2, Eno3, Fbp1, Fbp2, Fh1, G6pc, G6pc3, G6pdx, Galm, Gapdhs, Gbe1, Gck, Gpi1, Gsk3a, Gsk3b, Gys1, Gys2, H6pd, Hk2, Hk3, Idh1, Idh2, Idh3a, Idh3b, Idh3g, Mdh1, Mdh1b, Mdh2, Ogdh, Pck1, Pck2, Pcx, Pdha1, Pdhb, Pdk1, Pdk2, Pdk3, Pdk4, Pdp2, Pdpr, Pfk1, Pgam2, Pgk1, Pgk2, Pgm1, Pgm2, Pgm3, Phka1, Phkb, Phkg1, Phkg2, Pklr, Prps1, Prps111, Prps2, Pygl, Pygm, Rbks, Rpe, Rpia, Sdha, Sdhb, Sdhc, Sdhd, Sucla2, Suclg1, Suclg2, Taldo1, Tkt, Tpi1, Ugp2.
Fatty acid metabolism: Acaa1a, Acaa2, Acad10, Acad11, Acad9, Acadl, Acadm, Acads, Acadsb, Acadvl, Acat1, Acat2, Acot12, Acot2, Acot3, Acot6, Acot7, Acot8, Acot9, Acox1, Acox2, Acox3, Acsbg1, Acsbg2, Acs11, Acs13, Acs14, Acs15, Acs16, Acsm2, Acsm3, Acsm4, Acsm5, Aldh2, Bdh1, Bdh2, Cpt1a, Cpt1b, Cpt1c, Cpt2, Crat, Crot, Decr1, Decr2, Echs1, Eci2, Ehhadh, Fabp1, Fabp2, Fabp3, Fabp4, Fabp5, Fabp6, Gcdh, Gk2, Gpd1, Gpd2, Gk, Hadha, Hmgc1, Hmgcs1, Hmgcs2, Lipe, Lpl, Mcee, Mut, Oxct2a, Pecr, Ppa1, Prkaa1, Prkaa2, Prkab1, Prkab2, Prkaca, Prkacb, Prkag1, Prkag2, Prkag3, Slc27a1, Slc27a2, Slc27a3, Slc27a4, Slc27a5, Slc27a6.

To assess more comprehensively the expression of metabolic enzymes, the inventors performed a microarray analysis on kidneys collected at P10 derived from a hypomorphic Pkd1 mutant mouse (Pkd1^{V/V}), which results in a milder PKD phenotype⁴⁷. First, data were analyzed by PCA ad HCA, showing a very good separation of the samples in both assays (FIGS. 11D and 11E). Next, microarrays data have been analysed by means of the Significant Analysis of Microarrays (SAM) algorithm to identify differentially expressed genes⁴⁸ followed by analysis of the full list of genes (Table I) involved in GLY, PPP, and FAS showing that these genes are mostly upregulated, while the genes involved in OXPHOS and FAO are markedly downregulated (FIG. 6C).

Next, to validate our findings in human samples, the inventors applied again the SAM algorithm⁴⁸ to a previously published dataset of microarrays derived from the cystic kidneys of patients carrying PKD1 mutations. Data were next screened for the complete list of genes involved in bioenergetics (see methods) (FIG. 6D)⁴¹. The results showed that the metabolic alterations described in cellular and animal models of PKD (FIGS. 1, 2 and 3) and recapitulated by the mathematical model (FIGS. 6A and 6B) and by the murine microarrays (FIG. 6C) are all perturbed in ADPKD kidneys including GLY, PPP, oxidative TCA cycle (TCA/OXPHOS), FAS, and FAO. Importantly, in human samples as well GLY, PPP, and FAS appear to be mostly upregulated, even if some isoforms of the enzymes are downregulated. In contrast, both the OXPHOS and FAO enzymes are markedly downregulated (FIG. 6D).

These data taken together show that a general metabolic reprogramming of bioenergetic pathways is a hallmark of PKD and that likely most alterations are highly coordinated and occur simultaneously, opening unique opportunities for targeting them all with a few interventions.

### Example 6

### Inhibition of ATF4

Once activated, ATF4 binds to the promoter of target genes, increasing their expression^{49,50}, in particular, ATF4 binds to the promoter of ASNS increasing its expression (FIG. 12A,⁵⁰). ATF 4 was increased by 1,08 fold (P=3,21.10⁻⁷) in cystic kidneys from patients of ADPKD (FIG. 12B). Quantitative RT-PCR shows that the target gene of ATF4, Asns is significant increased in Pkd1^{-/-} MEFs and Ksp-cre; Pkd1^{-/flox} cystic kidneys at P4 compared to their relative controls (FIG. 10A). Microarrays from Pkd1^{V/V} P10 kidneys and human-derived microarrays of PKD patients samples have shown that ASNS was also significantly increased kidneys from ADPKD patients and mutants compared to the relative controls (FIG. 10A).

Overall, enhanced ATF4 activity might be a mechanism leading to the increased ASNS expression in PKD. Treatment of a murine model of PKD with an inhibitor of ATF4 may slow down the progression of cystogenesis. Therefore inhibition of ATF4 activity offer a good therapeutic strategy for the treatment of disorders characterized by renal and/or liver cyst formation, such as ADPKD.

In conclusion, in the present invention, the inventors performed a thorough analysis of the metabolic derangements observed in ADPKD using studies that range from global profiling in orthologous animal models to in silico flux analysis, in vitro carbon and nitrogen tracing, and finally validation in murine and human microarray datasets. The five main conclusions of the present data are: i) a global metabolic reprogramming occurs in ADPKD, involving several pathways; ii) of all the metabolic alterations, rewiring of central carbon metabolism is the most prominent and includes interlinked alterations of increased GLY, PPP, and FAS along with decreased OXPHOS and FAO; iii) glutaminolysis is enhanced as a compensatory mechanism and used for both energy yielding purposes and anabolic needs; iv) usage of glutamine in PKD is interlinked with the asparagine metabolism the asparagine synthetase enzyme (Asns); v) targeting Asns is lethal in PKD cells when associated with glucose deprivation.

Based on previous studies it appeared that minimal changes in a few individual metabolic pathways might occur in ADPKD. Here, the inventors show instead that the metabolic derangement in PKD tissues is rather robust and occurs in multiple pathways. In addition to GLY and FAO, the inventors find here alterations in the PPP, in FAO, in Glutaminolysis and in OXPHOS. Furthermore, the inventors show here that not only multiple metabolic pathways are occurring simultaneously, but that they are interconnected and likely causally linked.

In the present invention, the inventors have used a kidney-specific Cre line to inactivate the Pkd1 gene exclusively in the distal tubules and in collecting ducts (cadherin16-Cre, KspCre). This animal model develops a rapidly progressive phenotype with manifestation in the neonatal life. Further, the animal model used in the current study shows reduced variability in the renal phenotype allowing for a good separation of the sample by PCA analysis and hierarchical clustering. The present invention reveals that a much broader metabolic derangement than previously appreciated is present in PKD.

Importantly, the inventors analysed renal samples at the cystic stage. Therefore, the inventors cannot exclude that the metabolic alterations are secondary to cyst expansion. Relevant to this is the fact that recent investigations have unveiled an important correlation between chronic kidney disease (CKD) and metabolic derangements⁵¹. Here the inventors have excluded that at the time of analysis (P4) the animals are reaching CKD. Indeed, the inventors observed a minimal initial increase in circulating urea, but absence of prominent inflammatory infiltrate or fibrosis. In addition, most of the metabolic alterations can be observed in isolated cells. Likewise, the inventors have excluded that a prominent hypoxic state is present in the kidneys at the stages analysed. All these pieces of evidence suggest that the observed metabolic derangements are not secondary to CKD or hypoxia. However, based on the current knowledge the inventors cannot exclude that, as disease progresses, additional alterations such as hypoxia and/or CKD can further contribute to disease worsening through additional metabolic stress.

In a recent elegant study, Hajarnis et al has shown that the reduced FAO is secondary to the upregulation of microRNA-17, which in turn downregulates the expression levels of Pparα, ultimately reducing FAO⁴⁶. Of interest, the authors were able to rescue the phenotype of Pkd1 mutant mice by using fenofibrate, a natural compound acting as an agonist of Pparα and achieving enhancement of beta-oxidation and OXPHOS^{46,52}. Notably, the authors showed that the oncogene Myc is the main driver of miRNA17 expression⁴⁶. It is important to note here that Myc is indeed considered a master regulator of metabolism in several types of cancer, with glycolysis and glutaminolysis both being regulated by this oncogene⁵³.

The inventors found decreased levels of most glycolytic intermediates in the static analysis of KspCre;Pkd1^{flox/-}, including lactate.

Further, the invention shows that Pkd1^{-/-} cells uptake large amounts of glucose used in the glycolytic pathway to generate lactate and that only minimal amounts of glucose are fueled into the TCA cycle in mitochondria. Furthermore, the inventors found that an increased uptake of glutamine is occurring Pkd1^{-/-} cells as compared to the controls and it is oxidised into the TCA cycle, likely compensating for the reduced usage of glucose. Since the overall respiration of the cells is diminished, the likely explanation is that glutamine is used by these cells to preserve the mitochondrial membrane potential and avoid undergoing apoptosis.

Moreover, the inventors show that in addition to being oxidised in the TCA, glutamine is also used reductively in the Pkd1^{-/-} cells to generate citrate, which is transported into the cytosol and converted to acetyl-CoA, an essential substrate for fatty acids biosynthesis. The last process is upregulated in ADPKD likely to generate the membranes required for the proliferation of these cells. Indeed, silencing of the central enzyme Fasn greatly impacts on cell proliferation and survival. Thus, the present results demonstrate a critical role for glutamine as a compensatory mechanism for the reduced usage of glucose in Polycystic Kidney Disease. The inventors did not detect changes in the expression levels of the enzyme GLS. The inventors have shown here that the utilization of glutamine in PKD is interlinked with the synthesis of asparagine via the enzyme asparagine synthetase, ASNS, a transaminase that converts aspartate into asparagine while deaminating glutamine to form glutamate⁵⁰. Then, in PKD, glutamine is used as a carbon source to fuel TCA. Indeed, silencing of ASNS resulted in a complete rescue of the accumulation of α-KG, specifically by reducing the glutamine contribution to α-KG generation. Based on this, the inventors propose that inhibiting ASNS would be a much more specific way to reduce glutamine usage in PKD, opening an important novel opportunity for a more targeted approach in ADPKD. In line with this, the inventors have found that silencing ASNS impacts on the growth of Pkd1^{-/-} cells and this is more prominent when cells are also deprived from glucose. The data indicates that indeed glutamine usage compensates for the lack of glucose utilization in the TCA cycle by the Pkd1^{-/-} cells and that targeting both processes at once is more effective than either one alone. In line with this, the starvation from both glucose and glutamine drastically enhances cell death in cells lacking the Pkd1 gene. Thus, inhibitors of Asns along with a glycolytic inhibitor, such as 2-deoxy-D-glucose^{11,12} offer a good therapeutic strategy in the treatment of a disorder characterized by renal and/or liver cyst formation.

In conclusion, the inventors report here the first broad overview of the metabolic derangement observed in PKD. Importantly, the altered pathways that the inventors report in the current study expand the view on the potential use of inhibitors able to tackle the metabolic alterations to retard disease progression. The highly coordinated alterations observed offer a unique opportunity for targeting the process at multiple levels to block at once the capability of ADPKD cells to produce energy and to synthesize the building blocks needed for proliferation and survival.

### Example 7

### LNAs Gapmers

### In Vitro LNAs Testing in Pkd1-Mutant Cells

LNA gapmers were designed and supplied by Exiqon, Qiagen as >85% pure. The inventors have screened for 5 LNAs and tested efficiency in Pkd1-mutant cells and controls. To test efficiency of LNAs against Asns, the inventors delivered LNAs, in the presence of a transfection reagent, for one day at a final concentration of 50 nM.

Pkd1^{-/-} MEFs and controls cells were screened for 5 LNAs (#1, #2, #3, #4 and #5) and the inventors identified top candidates based on relative potency for reducing Asns expression levels (FIG. 13A) relative to mock treated cells. mRNA expression was measured by RT-PCR and the levels of Asns were reduced in all targets selected (Table V). Metastasis Associated Lung Adenocarcinoma Transcript 1 (Malat1) was used as a positive control. The best candidate targets were #1, #2, #3 and #5. To confirm, the downregulation of Asns, the inventors used #1 and 2 for RT-PCR and cell counts performed in Pkd1^{-/-} mutants cells.

TABLE V
Percentage efficiency respect
to mock treated cells.

| | % efficiency |
|---|---|
| Target | respect to mock |
| #1 | 87% |
| #2 | 80% |
| #3 | 81% |
| #4 | 48% |
| #5 | 77% |

Pkd1^{-/-} mutant cells show a reduced cell count when silenced with #1 and #2 compared to mock cells (FIG. 14B). Representative images were taken for each condition showing decreased number of cells in #1 and #2 (FIG. 14A) compared to mock cells. mRNA fold change for Asns was statistically downregulated (FIG. 14C, Table VI) respective to controls cells.

TABLE VI
Percentage efficiency respect
to mock treated cells.

| | % efficiency |
|---|---|
| Target | respect to mock |
| #1 | 87% |
| #2 | 80% |

The inventors then tested LNAs in another type of cells from epithelial origin. The inventors tested LNAs #1,2 and 3 in Pkd1-mutant mouse cortical collecting duct (mCCD) cells compared to mock controls. Representative images were taken for each condition showing decreased number of cells in #1, #2 and #3 (FIG. 15A) compared to mock cells. mRNA fold change for Asns was statistically downregulated (FIG. 15B, Table VII) respective to controls cells.

Based on the in vitro results, LNAs #2 was selected as the optimum target to test in in vivo murine model of PKD. Preliminary data show that at a dose of 30 mg/kg for at least 5 weeks, the mice show clear signs of toxicity. Other studies with a lower dosage are currently ongoing.

TABLE VII
Percentage efficiency respect
to mock treated cells.

| | % efficiency |
|---|---|
| Target | respect to mock |
| #1 | 92% |
| #2 | 94% |
| #3 | 85% |

### Example 8

### Results for ASOs In Vivo

The inventors tested in vivo anti-sense oligonucleotides (ASOs):

| SEQ ID NO: 33 | |
|---|---|
| ASO#1320551 | CCAAAATATGTGTCCA |
| SEQ ID NO: 34 | |
| ASO#1320506 | TATTTTATCACACTCC |
| SEQ ID NO: 35 | |
| ASO#1320496 | GCTTTTAAATGGTCTT |

ASO #1320506 was the most potent tested ASO and very well tolerated in in vivo screening.

As the efficacy/tolerability study was dosed at 50 mg/kg/wk, the inventors tested the same dose in vivo via i.p. injection. The study also included a non-targeting, control ASO (ACGATAACGGTCAGTA SEQ ID NO:36). The inventors show that in an orthologous mouse model of PKD the administration of ASO #1320506 resulted in an efficient downregulation of Asns with no obvious signs of toxicity.

### Efficient Downregulation of Asns in an Orthologous Mouse Model of PKD

Cystic mice (Pkd1^{ΔC/flox}TmCre) or controls (Pkd1^{flox/+} TmCre; Pkd1^{flox/+} and Pkd1^{ΔC/flox})⁴² were used to test Asns downregulation. Tamoxifen was injected at P24-P25 and the kidneys, liver and plasma were collected at P88-93. Control animals were also injected with Tamoxifen at the indicated times (FIG. 16A and FIG. 19).

To assess the Asns knockdown efficacy of the selected ASOs in vivo and to potentially rescue the cystic phenotype, 50 mg of ASO #1320506 were injected weekly of a 3.5 week-old (P23-24) cystic mice and controls. The same dose of ASOcontrol was injected. As shown before by our group, Chiaravalli et al. 2016, the Pkd1^{ΔC/flox} TmCre (cystic mice, will be referred) develop cystic, enlarged kidneys at 1 month after Tamoxifen injection⁴².

After treatment with ASO #1320506 (ASO #1 will be now referred) treated for a weekly injection of a total of 7 per week (except litter 4 a total of 6 per week, please refer to supplementary information), in the cystic mice treated with ASO #1, the kidneys appeared smaller than the cystic mice treated with ASOcontrols (FIG. 16B). The percentage of the kidney to body weight ratio was significantly decreased compared to the ASOcontrols treated animals (FIG. 16C). This is suggestive of a marked decreased in the expansion of renal cysts. Analysis of BUN, at the end-point of the study revealed that the functionality of the kidneys was improved in the ASO #1 treated animals compared to the ASOcontrols (FIG. 16D).

Furthermore, the ASNS mRNA expression levels were statistically reduced in all ASO #1 treated animals compared and as shown in Table VIII and FIG. 17.

TABLE VIII
Percentage efficiency respect
to ASOcontrol treated
(Pkd1^{ΔC/flox}TmCre) animals.

| Litter | % efficiency |
|---|---|
| #1 | 81% |
| #2 | 62% |
| #3 | 80% |
| #4 | 83% |

Liver toxicity was assessed by ALT and there was no statistical different in the cystic treated animals (FIG. 18).

### Example 9

### Small Molecule Inhibitors of Asparagine Synthase in the Treatment of Autosomal Dominant Polycystic Kidney Disease.

The compound (2R,3S,4R)-1-[(2S)-2-aminobutanoyl]-N-benzhydryl-4-(4-fluorophenyl)-3-hydroxy-pyrrolidine-2-carboxamide, herein referred as compound (cpd) 17, has the following chemical structure:

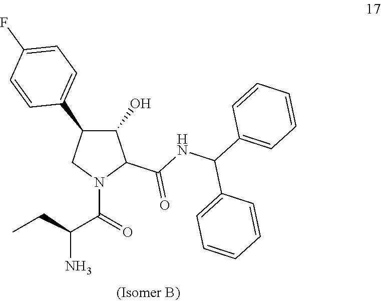 [SMILES: CC[C@H](N)C(=O)N1C[C@@H](c2ccc(F)cc2)[C@H](O)C1C(=O)NC(c1ccccc1)c1ccccc1]

The compound, which forms part of the invention, has been tested as a small molecule inhibitor of asparagine synthase in the experiments detailed below.

### In Vitro Data

### Inhibition of ASNS Selectively Reduces Growth of Pkd1−/− Mouse Embryonic Fibroblasts (MEFs)

Mouse embryonic fibroblasts (MEFs) lacking the Pkd1 gene show many of the metabolic changes also observed in mouse models for ADPKD, including increased glycolysis (Podrini et al. 2018). To examine the impact of pharmacological inhibition of ASNS in these cells, WT and Pkd1−/− MEFs (grown under standard cell culture conditions) were treated for 72 h with different concentrations of cpd 17, and the effect on cellular growth was established using an Incucyte live cell imaging device (FIG. 20-22). Interestingly, cpd 17 reduced proliferation in Pkd1−/− MEFs, while WT cells were unaffected. Taken together, these data showed pharmacological inhibition of ASNS in an in vitro model for ADPKD that is dependent on ASNS for its survival and proliferation.

### In Vivo Data

### Pharmacokinetic Profile

The Inventors subsequently determined the pharmacokinetic profile of cpd 17 in WT C57BL/6 mice after single dose administration (FIG. 23). Dose-dependent plasma exposures were observed (I.P. administration). Moderate clearance was observed (44 mL/min/kg) with a large volume of distribution (10 L/kg). Bioavailability was excellent after oral administration (92%) and intraperitoneal (I.P.) dosing (133%). In addition, mouse plasma protein binding (PPB) data showed a reasonable free fraction (˜5.5%). I.P. dosing of >10 mg/kg provides coverage of PPB-corrected IC50 (˜2 μM) based on in vitro IC50 values in Asn-deprived cancer cells, at least for some time. In addition, the compound was well distributed to the kidney (at 10 mg/kg and 30 mg/kg).

### Target Engagement Study

To test the cpd 17 in a mouse model of ADPKD, the Inventors took advantage of a Tam-Cre;Pkd^{IΔC/flox} mouse model carrying a tamoxifen-inducible and ubiquitously expressed Cre, enabling Pkd1 inactivation at different timepoints. Animals were injected with tamoxifen (200 mg/kg) at P11 and treated with cpd 17 (30 mg/kg) by intraperitoneal injection for 5 days on/2 days off, for 2 weeks (FIG. 24-25). Kidneys were collected and the concentration of Cpd 17 in the kidneys was determined as demonstrated in FIG. 25.

### Human Equivalent Dose (HED)

Based on the drug exposure in mice determined from PK, the effective concentrations of cpd17 in MEFs, ranging from 0.3 μM to 30 μM, are equivalent to dose levels ranging from 1.0 mg/kg to about 50 mg/kg in mice. By using U.S. Food and Drug Administration (FDA)-recommended allometric dose conversion tables that are based on relative body surface, the anticipated human dose levels that would achieve equivalent exposure to mice range from 0.08 mg/kg to 4 mg/kg. The range of doses to be used in humans is shown in Table 1.

TABLE 1
Dose levels in MEFs, mouse models, and Human Equivalent Dose (HED).

| Dose levels in MEF cells | Dose levels in mice | HED |
|---|---|---|
| 0.1-1 μM | 0.3-3 mg/kg | 0.02-0.2 mg/kg |
| 1-10 μM | 3-30 mg/kg | 0.2-2.4 mg/kg |
| 10-50 μM | 30-150 mg/kg | 2.4-12 mg/kg |

Some preferred maximum dose ranges for the invention are 30 mg/kg, 5 mg/kg, and 0.5 mg/kg. Some minimum dose ranges for the invention are 0.1 mg/kg, 0.01 mg/kg, and 0.001 mg/kg. Each of these three maximum doses can be combined with each of the three minimum doses to form a range of doses in the method of the invention.

### BIBLIOGRAPHIC REFERENCES

1. Torres, V. E., Harris, P. C. & Pirson, Y. Autosomal dominant polycystic kidney disease. Lancet 369, 1287-1301 (2007).
2. Aad, G., et al. Search for new particles in two-jet final states in 7 TeV proton-proton collisions with the ATLAS detector at the LHC. Phys Rev Lett 105, 161801 (2010).
3. Harris, P. C. & Torres, V. E. Genetic mechanisms and signaling pathways in autosomal dominant polycystic kidney disease. J Clin Invest 124, 2315-2324 (2014).
4. Ong, A. C. & Harris, P. C. A polycystin-centric view of cyst formation and disease: the polycystins revisited. Kidney Int 88, 699-710 (2015).
5. Qian, F., et al. Cleavage of polycystin-1 requires the receptor for egg jelly domain and is disrupted by human autosomal-dominant polycystic kidney disease 1-associated mutations. Proc Natl Acad Sci USA 99, 16981-16986 (2002).
6. Chauvet, V., et al. Mechanical stimuli induce cleavage and nuclear translocation of the polycystin-1 C terminus. J Clin Invest 114, 1433-1443 (2004).
7. Lin, C. C., et al. A cleavage product of Polycystin-1 is a mitochondrial matrix protein that affects mitochondria morphology and function when heterologously expressed. Sci Rep 8, 2743 (2018).
8. Padovano, V., et al. The polycystins are modulated by cellular oxygen-sensing pathways and regulate mitochondrial function. Mol Biol Cell 28, 261-269 (2017).
9. Qian, F., Watnick, T. J., Onuchic, L. F. & Germino, G. G. The molecular basis of focal cyst formation in human autosomal dominant polycystic kidney disease type I. Cell 87, 979-987 (1996).
10. Menezes, L. F., et al. Network analysis of a Pkd1-mouse model of autosomal dominant polycystic kidney disease identifies HNF4alpha as a disease modifier. PLOS Genet 8, e1003053 (2012).
11. Rowe, I., et al. Defective glucose metabolism in polycystic kidney disease identifies a new therapeutic strategy. Nat Med 19, 488-493 (2013).
12. Chiaravalli, M., et al. 2-Deoxy-d-Glucose Ameliorates PKD Progression. J Am Soc Nephrol (2015).
13. Taylor, S. L., et al. A metabolomics approach using juvenile cystic mice to identify urinary biomarkers and altered pathways in polycystic kidney disease. Am J Physiol-Renal 298, F909-F922 (2010).
14. Natoli, T. A., et al. Inhibition of glucosylceramide accumulation results in effective blockade of polycystic kidney disease in mouse models. Nature Medicine 16, 788-U787 (2010).
15. Menezes, L. F., Lin, C. C., Zhou, F. & Germino, G. G. Fatty Acid Oxidation is Impaired in An Orthologous Mouse Model of Autosomal Dominant Polycystic Kidney Disease. Ebiomedicine 5, 183-192 (2016).
16. chapman, A. B. R.-O., M. D.; Bennet, W. M.;. Treatment of autosomal dominant polycystic kidney disease. in http colon forward slash forward slash www dot uptodate dot com forward slash contents/course-and-treatment-of-autosomal-dominant-polycystic-kidney-disease?source=search_result (2018).
17. Walz, G., et al. Everolimus in patients with autosomal dominant polycystic kidney disease. N Engl J Med 363, 830-840 (2010).
18. Serra, A. L., et al. Sirolimus and kidney growth in autosomal dominant polycystic kidney disease. N Engl J Med 363, 820-829 (2010).
19. Yu, A. S. L., El-Ters, M. & Winklhofer, F. T. Clinical Trials in Autosomal Dominant Polycystic Kidney Disease. in Polycystic Kidney Disease (ed. Li, X.) (Brisbane (AU), 2015).
20. Schrier, R. W., et al. Blood pressure in early autosomal dominant polycystic kidney disease. N Engl J Med 371, 2255-2266 (2014).
21. Torres, V. E., et al. Angiotensin blockade in late autosomal dominant polycystic kidney disease. N Engl J Med 371, 2267-2276 (2014).
22. Seliger, S. L., et al. A Randomized Clinical Trial of Metformin to Treat Autosomal Dominant Polycystic Kidney Disease. Am J Nephrol 47, 352-360 (2018).
23. Magistroni, R. & Boletta, A. Defective glycolysis and the use of 2-deoxy-D-glucose in polycystic kidney disease: from animal models to humans. J Nephrol 30, 511-519 (2017).
24. Karaki, S. P., C.; Rocchi P. Antisense Therapy, A Novel Developing Targeting Therapy, (2019).
25. Gutierrez, J. A., et al. An inhibitor of human asparagine synthetase suppresses proliferation of an L-asparaginase-resistant leukemia cell line. Chem Biol 13, 1339-1347 (2006).
26. Larsen, T. M., et al. Three-dimensional structure of Escherichia coli asparagine synthetase B: a short journey from substrate to product. Biochemistry 38, 16146-16157 (1999).
27. Meyer, M. E., Gutierrez, J. A., Raushel, F. M. & Richards, N. G. A conserved glutamate controls the commitment to acyl-adenylate formation in asparagine synthetase. Biochemistry 49, 9391-9401 (2010).
28. Ye, J., et al. GCN2 sustains mTORC1 suppression upon amino acid deprivation by inducing Sestrin2. Genes Dev 29, 2331-2336 (2015).
29. Ikeuchi, H., et al. A sulfoximine-based inhibitor of human asparagine synthetase kills L-asparaginase-resistant leukemia cells. Bioorg Med Chem 20, 5915-5927 (2012).
30. Zhang, J. S., et al. Natural fatty acid synthase inhibitors as potent therapeutic agents for cancers: A review. Pharm Biol 54, 1919-1925 (2016).
31. Ruetschi, U., et al. Mutations in the 4-hydroxyphenylpyruvate dioxygenase gene (HPD) in patients with tyrosinemia type III. Hum Genet 106, 654-662 (2000).
32. Wodarczyk, C., et al. A novel mouse model reveals that polycystin-1 deficiency in ependyma and choroid plexus results in dysfunctional cilia and hydrocephalus. PLOS One 4, e7137 (2009).
33. Shao, X., Somlo, S. & Igarashi, P. Epithelial-specific Cre/lox recombination in the developing kidney and genitourinary tract. J Am Soc Nephrol 13, 1837-1846 (2002).
34. Dehaven, C. D., Evans, A. M., Dai, H. & Lawton, K. A. Organization of GC/MS and LC/MS metabolomics data into chemical libraries. J Cheminform 2, 9 (2010).
35. Surma, M. A., et al. An automated shotgun lipidomics platform for high throughput, comprehensive, and quantitative analysis of blood plasma intact lipids. Eur J Lipid Sci Technol 117, 1540-1549 (2015).
36. Distefano, G., et al. Polycystin-1 regulates extracellular signal-regulated kinase-dependent phosphorylation of tuberin to control cell size through mTOR and its downstream effectors S6K and 4EBP1. Mol Cell Biol 29, 2359-2371 (2009).
37 Mackay, G. M., Zheng, L., van den Broek, N. J. & Gottlieb, E. Analysis of Cell Metabolism Using LC-MS and Isotope Tracers. Methods Enzymol 561, 171-196 (2015).
38. Subramanian, A., et al. Gene set enrichment analysis: a knowledge-based approach for interpreting genome-wide expression profiles. Proc Natl Acad Sci USA 102, 15545-15550 (2005).
39. Kanehisa, M. & Goto, S. KEGG: kyoto encyclopedia of genes and genomes. Nucleic Acids Res 28, 27-30 (2000).
40. Pagliarini, R., et al. In Silico Modeling of Liver Metabolism in a Human Disease Reveals a Key Enzyme for Histidine and Histamine Homeostasis. Cell Rep 15, 2292-2300 (2016).
41. Song, X., et al. Systems biology of autosomal dominant polycystic kidney disease (ADPKD): computational identification of gene expression pathways and integrated regulatory networks. Hum Mol Genet 18, 2328-2343 (2009).
42. Chiaravalli, M., et al. 2-Deoxy-d-Glucose Ameliorates PKD Progression. J Am Soc Nephrol 27, 1958-1969 (2016).
43. Leroy, V., et al. Aldosterone activates NF-kappaB in the collecting duct. J Am Soc Nephrol 20, 131-144 (2009).
44. Naz, S., Vallejo, M., Garcia, A. & Barbas, C. Method validation strategies involved in non-targeted metabolomics. J Chromatogr A 1353, 99-105 (2014).
45. Castelli, M., et al. Polycystin-1 binds Par3/aPKC and controls convergent extension during renal tubular morphogenesis. Nat Commun 4, 2658 (2013).
46. Hajarnis, S., et al. microRNA-17 family promotes polycystic kidney disease progression through modulation of mitochondrial metabolism. Nat Commun 8, 14395 (2017).
47. Yu, S., et al. Essential role of cleavage of Polycystin-1 at G protein-coupled receptor proteolytic site for kidney tubular structure. Proc Natl Acad Sci USA 104, 18688-18693 (2007).
48. Tusher, V. G., Tibshirani, R. & Chu, G. Significance analysis of microarrays applied to the ionizing radiation response. Proc Natl Acad Sci USA 98, 5116-5121 (2001).
49. Pakos-Zebrucka, K., et al. The integrated stress response. EMBO Rep 17, 1374-1395 (2016).
50. Lomelino, C. L., Andring, J. T., McKenna, R. & Kilberg, M. S. Asparagine synthetase: Function, structure, and role in disease. J Biol Chem 292, 19952-19958 (2017).
51. Kang, H. M., et al. Defective fatty acid oxidation in renal tubular epithelial cells has a key role in kidney fibrosis development. Nat Med 21, 37-46 (2015).
52. Lakhia, R., et al. PPARalpha agonist fenofibrate enhances fatty acid beta-oxidation and attenuates polycystic kidney and liver disease in mice. Am J Physiol Renal Physiol 314, F122-F131 (2018).
53. Dejure, F. R. & Eilers, M. MYC and tumor metabolism: chicken and egg. EMBO J 36, 3409-3420 (2017).

1. A method for the treatment of autosomal dominant polycystic kidney disease (ADPKD) comprising administering an inhibitor of asparagine synthase to a patient in need thereof wherein the inhibitor of asparagine synthase is selected from the group consisting of: an oligonucleotide, a small molecule, an organic inhibitor, and an antibody.

2. The method according to claim 1 wherein the inhibitor of asparagine synthase is a siRNA or antisense oligonucleotide (ASO).

3. The method according to claim 1 wherein the inhibitor of asparagine synthase is an inhibitor of ATF4.

4. The method according to claim 1 wherein the inhibitor of asparagine synthase is a LNA.

5. The method according to claim 1 wherein the inhibitor of asparagine synthase is administered in combination with an inhibitor of glycolysis.

6. The method according to claim 5 wherein the inhibitor of glycolysis is a glucose analogue selected from the group consisting of 2-deoxy-D-glucose (2DG), SB-204990, 3-bromopyruvate (3-BrPA), 3-BrOP, 5-thioglucose, mannose, galactose, gulose, a 2DG having a fluorine in place of a hydrogen at any position on the glucose ring, a 2DG having an amino group in place of a hydroxyl group at any position on the glucose ring other than the 6 position, 2-F-mannose, 2-mannosamine, 2-deoxygalactose, 2-F-deoxygalactose, a di, tri, and other oligosaccharides that contain one or more of the preceding 2DG analogs.

7. The method according to claim 1 further comprising administering an inhibitor of glycolysis.

8. The method according to claim 1 further comprising administering an inhibitor of fatty acid synthase.

9. The method according to claim 1 ADPKD is caused by a mutation in a PKD1 gene.

10. The method according to claim 1 wherein the inhibitor the inhibitor of asparagine synthase is a small molecule.

11. The method according to claim 1 wherein the inhibitor the inhibitor of asparagine synthase is (2R,3S,4R)-1-[(2S)-2-aminobutanoyl]-N-benzhydryl-4-(4-fluorophenyl)-3-hydroxy-pyrrolidine-2-carboxamide.


SEQUENCE LISTING (36 sequences)

SEQ ID NO: 1 - DNA, 22 bp, synthetic construct
  misc_feature 1..22: note = synthetic
  source 1..22: mol_type = other DNA
catctgcgacagtcgagttctg

SEQ ID NO: 2 - DNA, 22 bp, synthetic construct
  misc_feature 1..22: note = synthetic
  source 1..22: mol_type = other DNA
cacaacccatcgtgacattttc

SEQ ID NO: 3 - DNA, 20 bp, synthetic construct
  misc_feature 1..20: note = synthetic
  source 1..20: mol_type = other DNA
ctgtgcaggctgctgtaacg

SEQ ID NO: 4 - DNA, 20 bp, synthetic construct
  misc_feature 1..20: note = synthetic
  source 1..20: mol_type = other DNA
gttcccgaaaccctgaggag

SEQ ID NO: 5 - DNA, 20 bp, synthetic construct
  misc_feature 1..20: note = synthetic
  source 1..20: mol_type = other DNA
ttgctggcactacagaatgc

SEQ ID NO: 6 - DNA, 20 bp, synthetic construct
  misc_feature 1..20: note = synthetic
  source 1..20: mol_type = other DNA
aacagcctcagagcgacaat

SEQ ID NO: 7 - DNA, 20 bp, synthetic construct
  misc_feature 1..20: note = synthetic
  source 1..20: mol_type = other DNA
agtggcctcacagactccag

SEQ ID NO: 8 - DNA, 20 bp, synthetic construct
  misc_feature 1..20: note = synthetic
  source 1..20: mol_type = other DNA
gcccatgttgtacagcttcc

SEQ ID NO: 9 - DNA, 22 bp, synthetic construct
  misc_feature 1..22: note = synthetic
  source 1..22: mol_type = other DNA
cccaacttcttcaagatggtgg

SEQ ID NO: 10 - DNA, 21 bp, synthetic construct
  misc_feature 1..21: note = synthetic
  source 1..21: mol_type = other DNA
agaggctcaacacatggttgc

SEQ ID NO: 11 - DNA, 20 bp, synthetic construct
  misc_feature 1..20: note = synthetic
  source 1..20: mol_type = other DNA
ttcgccctcggagatcctac

SEQ ID NO: 12 - DNA, 21 bp, synthetic construct
  misc_feature 1..21: note = synthetic
  source 1..21: mol_type = other DNA
ccaagctaggtaacagaccct

SEQ ID NO: 13 - DNA, 20 bp, synthetic construct
  misc_feature 1..20: note = synthetic
  source 1..20: mol_type = other DNA
ggttttctcgatgcctcctt

SEQ ID NO: 14 - DNA, 21 bp, synthetic construct
  misc_feature 1..21: note = synthetic
  source 1..21: mol_type = other DNA
tgtggctctgttacaatggtg

SEQ ID NO: 15 - DNA, 20 bp, synthetic construct
  misc_feature 1..20: note = synthetic
  source 1..20: mol_type = other DNA
ttatgtcccccgttgactga

SEQ ID NO: 16 - DNA, 20 bp, synthetic construct
  misc_feature 1..20: note = synthetic
  source 1..20: mol_type = other DNA
acattgtggccctctgtgtg

SEQ ID NO: 17 - DNA, 21 bp, synthetic construct
  modified_base 1..21: mod_base = OTHER; note = uracil
  source 1..21: mol_type = other DNA
gggatcataaagataactttt

SEQ ID NO: 18 - DNA, 21 bp, synthetic construct
  misc_feature 1..21: note = synthetic
  source 1..21: mol_type = other DNA
  modified_base 1..21: mod_base = OTHER; note = uracil
aagttatctttatgatccctc

SEQ ID NO: 19 - DNA, 21 bp, synthetic construct
  misc_feature 1..21: note = synthetic
  source 1..21: mol_type = other DNA
  modified_base 1..21: mod_base = OTHER; note = uracil
ggcccttgtttaaagccattt

SEQ ID NO: 20 - DNA, 20 bp, synthetic construct
  misc_feature 1..20: note = synthetic
  source 1..20: mol_type = other DNA
  modified_base 1..20: mod_base = OTHER; note = uracil
atggtttaaacaagggcctg

SEQ ID NO: 21 - DNA, 16 bp, synthetic construct
  misc_feature 1..16: note = synthetic
  source 1..16: mol_type = other DNA
ctaactaaccaatgcg

SEQ ID NO: 22 - DNA, 16 bp, synthetic construct
  misc_feature 1..16: note = synthetic
  source 1..16: mol_type = other DNA
tagtcaaatgccaatc

SEQ ID NO: 23 - DNA, 16 bp, synthetic construct
  misc_feature 1..16: note = synthetic
  source 1..16: mol_type = other DNA
gttattgtaacgacga

SEQ ID NO: 24 - DNA, 16 bp, synthetic construct
  misc_feature 1..16: note = synthetic
  source 1..16: mol_type = other DNA
ttaattcttgacacgg

SEQ ID NO: 25 - DNA, 16 bp, synthetic construct
  misc_feature 1..16: note = synthetic
  source 1..16: mol_type = other DNA
ttctcctagctaacgt

SEQ ID NO: 26 - DNA, 18018 bp, Mus musculus
  source 1..18018: mol_type = genomic DNA
aagcggcctccaaccggtcttgtcactgcgctgcctctgctccaccttctctggccctggccgctagtgctcaggtaagcgggagccgggtgctcagcgcggagcgctctgctcgcctgggaggccggaggatgtgctgtccgcgcgggcacatgtcccgggagttcggatgcggagcgctcgggtccggcgccagaccgatgctgcggtgcagcgcgaggcagggcggggcgggcgcggggcgattgttgcgcacctaggacggagctgccaggaaagctttcatgtgagcgaagtgcagcgcggaggcagggccgctccagacagaaggcgaaggtggccttcatttagacaccataaatatttttttttactgtatgacgtctagatctgggaaatttcagccatccgaaggttttgcctgggactaggtggacgcttgcacaagaccagctgtaattgggagccccaggctagtgtaggggtctcgctctccagcaggacagaggctctaccaagctctagagcgagtgtctctgggtctgtctggatccggatttctgagcactcactcacctcgctgctcaggcccttcctccactccagttccatactcgcagccacgccgggaaaaatctgtttaaggagacaggcaaccaagtacgggaaaaggcaaaggaattggcgtgcttttttttttttttttttttttttcttaattatggatagctatttcacaggtctagtttcagataagataacttttaaaatacacgggcgagtttgatttttcgttgcctgtggcctacgggttttaacagctaactcttcgttgattgatgtcagtactataatattcatttgaagtaagacttgcacaaggccacgggttgattattgaggtcttaaggtgatccagtggagtttagaactatcagttcagtgtacgaaagcggcgctgtccagcggggagcctgtgctctttcatagactgacccctagaaatgctggctagtctgaactgagatgtggtaagagtgtaatgtgtcgatttcgatgattatgaaaaccgaacgcttgatgttttgatgtttgattgcagcctctaaccactaaagtatccctccagcccggttttaataatttaggaaaagtagtgtaaatataattaattttgattatattgtattggttgtcgtatttagggtgtgtcatgttaaggtatgtttaaaattagttttaatgttttctcttgtcttcttaatgagctttattagtatattaaaatttcacatatggctcactggccagtactgctgcagaagttaaccaggaaaggaggaaatttacagacaacagtgaggaaaatatggtacaagagggcctctgagtccgattacaaaggagtttgaatttccttactcctgctgaaggaaagaattttaaacttttctttacacttcaatgtagtgcctggcatatgttttgcataccagacagttcattaaagagccttaagacctaggtaaatggttctcggtgatcataattgattttcagagcgctttgttagcccacttccctttcccttttattagacatagattcttttctcatataatactatctgaacagtttcccacctctctacttatagtgtctccccacctctcccctctgaattcaccccttttctgcctctcattagaaaaggataggcttctaagaggcaagcaacaaaatagaataaaatacagtaaggtgagataaaagggaaaccatcatattgaggttggacaacagaaaccaacagaaagataaaaagatcccgaagagaaggcgcaggaatcagagacccggattcacagttcagtcaggagtcccataaacgcactcaactgaaagttagaatactgtgagttctcagccttcccgatgctgccgccctttaatacagaacactgaagtgaaccccagccataacatcatgttcctagctacttcatcacggcagctttgctactgttttgagtcatattgtaaatatgtaaatatgttttctgttggtcttaggcaaccacggaaagggccctttgaccccagggttgagaaccgctgctatagtacacatgcagaggacctagtggagacccgtgcaggccccacgcttgctgcttcggtctcggagttcatcggagctttgctcggttgttttaagaggaccttgttctgatgtcctctgtctcaggtttttgcaactccttctgcctcctctttctcagggttccctgagctctgagggtcgggattcgatggagccatcttttagcagtgcgactaacccacttttcataggcatcgaatcaaacactgcttattaatggccacttctcctatatctctggattctccttctgaatgcgcagaccgagcctatcacaggccatttggacagcatggaagggctatgggcctcttgcacttcctcttccggttgcattgcttgataccactttccgtggcttggtctggttttactcaaagcgcaccaaagctttgatccagtcggaatgtttcccaaggcttgctcagcgtggggggatgggagggtgtaggtttcagaggatgtggccatttgttgcagatttgctttttgaacaggattgcatgttcaaagactctttattgaactatttgaacttcagatatttaaatgaaggatttgtaggaattacttgcttcctggcttacagggctttaattaagtttccccccctccagtgttgtttctaactaaccaatgcggctttgagataaatatttgagagtgtttttgtcattaaggacactggatgttgaacttctctgttattgacacatggtctcatacagtacaggatggccttgaacctttgatctaccctgcttctaccctcagtgctagcattaagcactacagtacccagccatgacaaatattttacatcctcagtaaactcaaaatgcttgttagggtatctgatatcagattattgtttccttattcttccataacatttagcgaagtcacttagaggttaagctagcagagtgacatgcctaaggatgtattatactagtgtgtgtgtctctctgtgtatacacatgggttaatattacaaaatggtgcccacgagttgctgtttattccctttgtatttgacctggaaatagagagatggcatttaattatagaggcttaagaagaggctgtacgaacatggagaatctacctttggggtgttttgaggttctcacctggagtagctggggctggtatcccacctagcttggagccagctactcctgctattgctctttggagcatcctgggttgcagctgtgacctggatgttgctgggcgtaacccccaccaaaaatcctaaggtgggagaaatgtgagctttggctgggtgcagggtttatggttggatataaaagtcatttgtatttcttaatatttgtaagcaaatgaaatcactccccttgtgcctacccacctaatgggacattgcagtagataaagctggtttgtgtccaacatttggagagggttgtgtaggtttcaagaatccagagttgcttatactcaatgtgagaccctgtgcttctctctgtagagtgcctgcagtccgcctgtagcatgtgtggcatctgggccctcttcggcagcgatgactgcctttccgtgcagtgtctgagtgcgatgaagatcgcgcacagggggccagatgcatttcgctttgagaatgtcaatggatacaccaactgctgctttggctttcaccgcttggctgtggttgacccgctgtttggaatgcagccgataagagtgaggaaatacccttatttgtggctctgttacaatggtgaaatctacaaccacaaggcggtaagcagagtgaagctggggatggctggctggctataactcaccttcagacctatggttatgatgttcatattttgtgtcacgcttttttactttgtatctttttctatgttctgactcctttgctctggtcttatacagaaaactgacacagaaaggtaaaattagttagattttttttcaagtgattaacatggagatgccaattcaatttttttcctgttatacattttaatattgattagggcagtgttagctgatggagtcctgtcagcgaaaatgtcaggcctgtgctccattgcaggttacgctaacaggagacagacaaaaaaatcactgtaaattgaactttcggaaagcaaatgcagaagtggtgcacataggaaagcggtttgcggggatgggtctgaggaagggagacttaaactgagacctgatagacagagaatacttgccaagccccggacgtgtttcccatagagggggatagcgtgtgtagatgcccagtgtgggcgagagtgagtgagaagaggacccatggagctgggaagacaggacagtcagtctcctgctataggagccttccaaggccaggagcaattgagggcttagaggggagagatagtcctgcaatgttgagaggaggtgagatttgcagtgttatataatagggccactgagaaagcgtttgcagtttcacacttagtaggagccaaattgtagaaaggtggccggaggcactgaagtgtgttaagcaagacttgcagcattgaaactctctggtgtctagagaacaggagcgagtgaggggcagaaatgctgatctcggaaaccagatatggtacggaccatccaagccaggattatttgacttgtttttcataaggaaaaagaattaaatctttaatttatgagtgcttgtacaaataccaccccttgttatatttgcaaacttgttaacccctccccaacttctagttgcttcagagcaatgtgcctgagtactgggtattgttactgcctgtctgtatttatctccaggcctgcgtggtccctttaggctgaataattctagtatggtacaccataatgtgtttcctctaaaatatgtaatcatttgtcatttaataatcagaacattctcctagctaacgttaattcccaacctaagactataacatctaataatataataataatataatctaataatataatatctaatatcaagttcatagttaagcttctccaaatgtccctagaatacatttagctgtgtttttgtttggctttgcctttttgtttttatttttttggtgtgtgtgtgtgtgtgtgtgtgtgtgtgtgtgtgtgtgtgtgtgtgtgtgtgtgttttcatgcttatgcacccctgtttggttttgtttgtttggtagggcagatttcagtcaaggtttagtcatttggccatgctgtttcatggtaaattttgagacttctgtgcctatctttcattggtaggggtttatctttattcctggacaatggttttaatttgccagtttgcttgcttataggggcattgtcaaagcagggttctcatctgaagcctttaaccacttagcaaatataccttcataaagggcttctgtatgcaaagctcaatgctctaggatgaacccggtacagaggaaagttaaggaaagcagtgaggggtgaggtggagtttgggtctcagtaagtcaagccagtgtacctgagaggatgaggttgcgctctgtcagtatcacccttcatcttgtcttcctgaagtgaggttagcacatatcttggcttatggtttacttggacagtgttgctgctgcttattaattttgttaacaatttaaaaggcaatatcttaaaaacatgccttttaaaaattaggtaatgttatatttgcagagactttctgctgtttctaccaagcatccaaacttaaacaaatgcgactgctacaaacttgtctaagagtagaatgcacaacttcagccataagtatttttcagaatgcaccttcaaagttcgtcttcacctcctgcttataagccaggggcccagaacaagtccgaggccggtgtggctttaggaaggtggcaggacatgtgtgcagcacagaagcgtccattccagagttagagttagtactcggcagttgttagtgaagggacgagtgctttctgagaaaattatcattgaaaatgagagtggaagcgatgttcattattttcaaatgctgtagggtgctcccacccagaggggtagatttttggggttgaagagatgcctcagcagttgaatctgttcttccagaggtgcttagttcaattcccagcacacacatggtggctcgggaccatctagaaaagaaactgaagcatgtaggtgtacacactgcagaacactcacacataaaatcaataaagtaaacagaggggtagatttttcaaaccccacctacctgagccaggacgaccctgaccagtccttagcttggaagaatggccagccggtactgtaagagaggcagctaaagcacgtgttttaacagttctgtgaatgctttccacagaggaccacgcggtagggatggaaagacctaacgcgtggctggtgtgctagtgtagtagaaactgtcagaatctgggcccaactcaagccttttcactttgctgacagagacagtggagggcccattgctgttcctgtgcggcctatctatgatcattttctctgtccctgagaagtagctttaatatcccactattcaggaaggtctggctgttgtcctctcctgaggaccagcagcttgggtctgggctctgccgttgtgatggattcctttgcatttgtcagtaaggggtggccactaggttttccttttgctgaatatttcaagggaggtctttgaggattagctggaagggcctctcagccgattctgcatctcaaaccaaaagtcaacagttgttgataacacatttttgctccatggtaacaaggcaaactgttctccaagtgtgttggcatgtccttatctatattcttggtttctaccttcaaagatcagagcaaacactggtgggaaatgtttatgaaaaattatacctatgatgaacatgcccatgtctttacaccctgcacaatattgcataacaattgcatctaaagtgttgtattaagttttttttccccctttggttttttttcgagacagggtttctctgtatagccctggctgtcctggaactcactttgtagaccaggctggcctcgagctcagaaatccgcctgtctctgcctcccaagtgctgagattaaaggtgagcgccaccactgcttggcgtgttaagtattctaaatctagaaagcatttaagtgcacaggaggatgtgtattatattatatgtagataatattctatttcacaaaaagcactaggtcacctgcaggtttttgtcttggggctggggtcctaaagaaaggatgacttcccatgtaaatgtgtatgtgtaactacagagtgtagactctgggctgcaatatgtacttgaatttcttcatgtgtcacagctctgatgcctgacccttgtatcctgtttatcagaacacagtctttatcgtcatgggtttagcctttatggtgaaaatttttcagaattgtgagtttagacaaggtagatttgaataatgccatacttttttttttctttctccttcttcttcttttttttaaatcactaatgcacacgagctgatgctagaacacagatactgggtcagcatccataaggatactaactttttgtttgctctgcttccttaagctacagcaacgttttgaatttgaatatcagaccaatgtggatggtgagattatcctccacctctatgacaaaggaggcatcgagaaaaccatctgtatgctggacggggtgtttgcattcatcttactggacactgccaataagaaagtatttctgggcagagacacctatggagtcaggcccttgtttaaagccatgacagaagatgggtttctggctgtgtgttcagaagctaaaggtaataatggagtttaactatttttggtttgtgtttcttcctggatcatagtgagaatcccagagaggtgtcaatcaaaaactttaaaatgatgaaagatgaatactcagtatgaataggatttgaaatcttagtcaatcctttcctctttcatgtgtgcgtgtgtgttgtatacacgtgtgctcatacatacgcacacgtagagaccagagacttaacattagacatggtaattttattttatttaatccgccttcctctgcctcctgagtaccaggattaaagatatgcaccatcgtgccagtctccattttaattcttgacacggtctctttgcacttagaactcactcactcacgttcctgatttagtggctgagctccaggtctccacctgccttcttcgtcctgccccacctatcctagaaataaagatgcaggatgctgctaccaggttctccatggggcctggggattaaatctcagttcctcaggcttgcttagcatggggcttggcaggcaccttattcactcagtctttctgcagcacccggttcttctggcctctaggaggtgagcacattttttcttcatgcaaagtatctctgccatggtttatagatagagccaagcaaccacagacttggtcacagtggctcaaatgcttgcgctaaaataatatttcttttacatttcttatcttgggtatattattatagcaatagataccttaacttgttatactcagtataatacacctacttagcattatttttctggcagtaaactttgtaatcatctttggtctcttacctagatagcaagttttctgtagtgtagtttgccattttttgaatcctttgccacttagtcttcagattttacattacttgaaaccaggatcttgatctgcatgctaaccaaagtgtgggtattaaatatgcttcccttggtctgtcagcagacagcatagggcattctacatcagttacctaaaatcccgtcagggaaacctggcaaaggaaatataaagcaggatggactcctgcttctgcgggctatcttttggacttttagacctgatgatggcttgatacggacctggaatcccaccagcagtttactctctgcagtttgttttctgcatccttaactctgtccttttctagactgcctagcgacctttacattactatgataagaaatcacgaccaagacaagactttatttggagcttagagtttcagagggctagggtctatgaggtcatgatggggaacatggaagcaggcaggcagcggctgggagcttacatcttttttttttttttaaagatttatttatttatttattatatgtaagtacactgtagctgtcctcagacattccagtcagatctcattacagatggttgtgagccaccatgtggttgctgggatttgaactcctgaccttcggaagagcagtcgggtgctcttacccactgagccatctcaccagcccccgggagcttacatcttgatccacaagcatgaggcagagagctgactgggaatgatgcgggcttttgaaacctcaaagcttattcctagtgatgcccctgtaagaaggccacacttcccaatccttcccaaacagttctattgactgggggccaagtggatatgctctgtaggggtcctattgaatccaccacacagatcccattggagtgctgcttccttaaccaggctttaaaaccatctcatcgtggtgatctgcctaagacctttcagttggctgcatcacatcctctatggggtgaacagcatttgagctctgccagctagtgagcacactgcattcctttgacctttgaattcaatgtacacagattgggttataagactcatagtacattataacaaatatctgtgtagatagcctgtttatctatttattacacaacattggcttttgtgcagatcacggtggtgtcagggtagctactgtctttaagaactgcctacagttacattctatggctttctcatacacattctatgacagccatgaaggggtgggtactggcttctgagtaccagtagtgtatcattttacgtggtaaagacttgagaaccattggtctctcaaaataccatgccagttaacatgaagttaacattctgatttctcttttttctcagatttatatttcctcttgtttatgttctgacagaaaataggccttccctttaaaaagcttttttctttgaaaactttataaatgtctaaaagacaggtttcagaaaattcagctaggtaaaggaatgggatgatgaagactttcattttaataaaggtatcagggcctttcaggtgggagagaggattgtctcagaggatgttattgtaacgacgagacatacactagttaaaaggtgatgtttggaatttttatcttgctatgttaggaaattatctcgtaaggaatcatcttcctattgcatcaaatgactgcaaatccatgctgttttgtgttctgtctccatgtcattcttgcttcttcaggagagatgctagggtatggggtaattatttcccctcccaccttttttgtataatggatatgcgtttgtacttaagggggaaagtatgggttagagtgtactagctcactgcacaggctgcagagaagcacctaattgcatacgttgctgttttccatttataggccttgtttccttaaaacactccaccactcccttcttaaaagtggagcccttccttcctggacactatgaagttttggatttaaaaccaaatggcaaagttgcgtctgtggaaatggtcaaataccatcactgtacggatgaaccattgcatgccatctatgacagcgtggagaaactcttcccaggtgactagtgcccatgaccaaaccccagaactctagctcacccctgacaactcagggccatttgcgccgttttcaatgatgttggttttaagatgttcctctttgatttggaggtggagcgatctccgtgagtgagggagagccatcacaatgatgtagtagactgcttgcctcatctcagtcagcttagcctcagcttgtttgtggcggttccatttactttctgtgttgttaggctttgacctagagaccgtgaagaacaatctgcgtatcctttttgacaacgctatcaagaaacgcttgatgacagaccggaggattggctgccttttatcaggtgaagtcactttaaaaaataaaaacatcttactggtgtttaaatgaaatgatttgtagctggcactgagctttgaatgttaaaggcctactatatagtttcatgtttcattttctatagacgtgtttataaactgcatttgttagggttagagcttgttggtcctcatcccctagactcctgtgtattgatcttttaagccagtttagatgcagttgccattgtaaggggacaagaaaatgtatgtattcacacacacatacacatacatatacatatgtatgtgtacatacatacacacatacatatatatatatatatatatatatatatatatatatatatacatacacacacacacagacacatacatatatacatacatacacacacacttcatttcacttcactataaatcttactaaagctgatttctgtttataaaactcagagatcttcatagtcaaatgccaatccagatgtttagcaggtgtgtgattatctttgccctgattaatgtatgcctgcaaccatcaagtttattttcatgctgcagggggcctggactcgagcttggttgctgcctctctgctgaagcaactcaaggaggcccaagttcagtatcctctccagacatttgctattggcatggaggacagccccgatctcctggccgctagaaaggtacagcacaacctgcccatcgtatagactcagaacttagcccaattgttaggacgaaaatcaagctgaagtcgtcctcgtgtgcctgttgtgttactatgagaaaggtcatgctagaaagcaacagggtgaggaacggattcacgtggcttacatatcccgagttgtagtccactgacgaaatctaggtgggaatttgaggcagaagcctgtaggcaggaactgcagcagagaccatggaggggcactgcttagttgttggtttgctcctcctggctcctgatccttcatttcccatcttgagttttctgctttgtctttcctccgtggactgtgactcagataaaccaaataaactctttcctttccaagttgatgttggtcacgatctttgtgacagacaagccatagagaacaactaggatagggactggtgccaggatatcagctcgcatcttgttttcttatgaagcatatgcattacaagcagatattgtacatgcaatgccgtgtgtggaagatggcgtgctgctctgtaaagacctagaatgactgagagaagcctcgaggagaattccaattcgacaggaaaaggctatatatgtagggataccatgctggatataaaattaggcatttcagtacagtacaagggagatgatggtcaaaagggagaaatgaggcaggttaagtaaaataggagagagtggtgtgctgattgggactctggactgtcagtctgtagctttgatttgaagagagccaatgtggttgagtggggagtgagggccaggcagaggcacctggcttgctttaggtagcatgtggctgtgcctgcatctatctcctcttacagaaggggcctgcacctcctcttattgaaaggcctgcaccgccccttaccaaggcggtctggagagaccttgagtcatcttcagctttgtttcttttcaccatgtttaccttgagaggaactggcctgtttcattttctaaagtggttctttaacaaatctatggagtttttcttcacaatcaggtggcaaattatattggaagcgagcatcatgaagtcctttttaactctgaagaaggcattcaggccctggatgaagtcatattttccttggaaacttatgatattacgacagttcgggcatctgtgggtaagggattttattatagggaaaagcgttttaatactgaaacagtagaaggtttaacatgatttcagcttaagaaatattgtctaaatatgaatactagcaaagttcctatattgttaataatactataatatagtgataagaccatttaaaagcgccctgaatttctttcgactggaggaagtgcttaatgaatataatcagatcatctgaacactgaatttacaaggtcagctcggcctgtggtcttaaagaatgcttcataaaaataaaagcagctcacatctgctaagcacatacttcatgctaggttctgtctcccatacttcatatgaattatttgttacctcagaactaactcatcataaccaggattccagcacccaggaggttgaggcagggggattgctgggctgtcagcatatggtatgaccatagaatggacacatattttggctccctttctctcatcttctctgtttaaaatatcttggatgatatagttgggccacagtacataacattattatgtataataatctggtgtgcttcctagtgcatgtaaaagctatggctacagtgtactatggtcaactaagtacctttatttaggaatatcttattgttgttgatcatctaagctgtggaaaatgatacagtgtatttgcttagtgcaggattgccagacttttcatttgtaagaaattaacttcctctgagatacaataaagctaagcatagtaaaacaagataggcctgtagatgtgtgtaggttgtgttcaaatgctatcattttacaaggagttgggtatctgaagatatgtgtagctgtttggttcccagaaccaatcccaaatgaataatgagggaacactgttcttaataaaatggggaaatcggtccatgttacagtacattttctctaacgggtcctcatgtagatgtataataagtattccatcacacagcttaccagttactagctattattgtcatgcaagcaacactacttctatctggcgaacacattctaagattagaaatgtaacacaaatcagacccaattttgttgaacctctaaaattttactagctgagtgagtccttttaaaaactcactttggagtgtgataaaataccttgggtattacagtgatttggtctcctgaagcatcatagttaaatactcacacgtatagcacatctgtggttaaatttttatgaggtggaagcagagttagaagaatgtctctcctaagaaggataaaaatgagattggaggagacagatggtagtgtagagagatggtagtgtagagctatctaccaagtagacatagcacaatattgtacagagccttcttaatcaaatgtgggatttggaaatgaggggtggaaatggaaaagaacgaaacaattaaaagtaagctatttccattagtctgctttctgttacaataatggataatgccagatgagaaactttagaaagcccaacagcatggcatgttggtgggtggcatcaggagtggcacatgagaaaggcatctcatagtgatcccaggcattggagacaagagatgggctctgtcctaaataattgaatcaggagaaatcatttgatgcttgaggagcaaggaaccccaaccacaccttatcttccacttctccaccatggatcacaccaccacatggagaccaaactgcatccaaagcattgcaaggccacgccttactctactgtgttagtacacatgccgtggtattcaggcatttgagtggttcaatccagcaaacatttaacatagaaagtcttgttctaagaacatagctttgcaacctgtttgtaaaacatgacagtggattgcaggctattttattgtttaggttggattgacagcttgtagatgacttacaaatttccgatccttttggaagctgctttaatacttatactaactcagtccaacaagacactccagcctactgttgcttctgaagacaacaatgacatcgccattgatttctctttcatttaaggcatgtatttaatttccaagtatattcggaagaacacagacagcgtggtgatcttctccggagaggggtcagatgaacttacacagggctatatatatttccacaaggtaagcgctctgaactgaaacgatggccaggctaagttaggaagtcagaatcacttgaccctttctattctgtccttatgatttgttgtagttttagagtgtatttgtgtttgtgtttaagaattgatgctatctatctgtctgtctgtccgtccgtccgtctgtctatctatatatttataggagaaagaagacttaatagtatcagatactttttattcttaaacacgagaattgcttttgtattttatctgtctgactgtctgtctattgatttttttttttcaagacagggtctcattaagtagctctggttgttgagaaactcactttatagaccagactggcctctgcttcccaagtgctgggatgcaaggcactgtgtctgttgcccagggcactcatttaatttgttttcacactaagcacttcggaggatggatgggtcggtcttgtaaaacattgctggcctctgaagtcatttactcacatcctataccctcctcttaagctttctgccttactcattctttatttcatatgtaagaggggaaaatgaaggctgcctgtgtgccaaaggaaggcactcaccttcgtttgttaaataggctccttcccctgagaaggccgaggaggagagtgagagactgctgaaggaactctacctgtttgatgttctccgggccgaccgcactactgctgcacacgggtaactttaaaatcagttgtaaaaccaacaagggagaagggggaagtagcttaaatgagatcagttgcttattttgagtttctttcggctggctgcaaagtatggttttcagcaaagtttgtcccctaaaagctgtgctatggcctggacactatcaccggggtgtggagagatcatgtcctgtgggtatgccaggcaggttttgtgtcactagatcataacagcaatgggccgtttctggcctagagggaaggctgcagcggcttcttggaaacattttagagcatttggagtattgcttctggtagcaaatactacgtgcctgccaagccagtttgctgcatctgggaggagaaatgcaatcttcatgcattcattcatttgttgataggactatagaataaaaataagtttctttggagccatttcttcttgcccagggtttctagggttaactgggcttgttgaagaatagaagagctttactgtgcaggataaagacttgcctttcaacattttcagttcctctactgaaccatatttggcttacgaatggtctggaatcatagttatgacctgcctgcatgtcctgacgagtgtcactgggacttttgtacatatgatggtatgaatattcatataccctcatgaccaggtcactcatgataattaacgttttatagaaacaggtaatttttcataggcgcagaaggaaagaaatactttaagacagaaagcttccctccctagatgctggttttatttcaagtacaaacattagtatgtggctaatcatgcagtgtggtccctaggaggctggaatgtaactctttcttctccatgactcagtctggggaggttgtcaggaagccgtgccagccatctgactcagattgtcttctgttttccccaagtctcgaactgagagtcccctttctggatcatcggttttcttcctattacctgtctctgccgccagatatgagaattccaaaagtaggtatatcaaccctggataaaacccccaaatattgttggttcctccggcgacttcactgaccctccttgtgtgctcctgtcagaatggcatagaaaaacatctcctgagagagacttttgaggactgcaacctgctacccaaagagattctctggcgacccaaagaagccttcagtgatgggatcacctcagtcaagaactcctggttcaagattttgcaggactatgttgaacatcaggtctgtctttaaagatatatattctggaacaataatagtttaatagagagctgggatctctgtttgcctttcgtttgaatagttgtatctaaaggagagtcagagttcctgcaaggaatatgtcttttataagctttatgtaataattaaggaaaatgaaggactataaagtagtattgatgatcaaaagaaaagggatagagggaagacttccaattcattgctgatcattttgcacgtctgggtacctgatgagtacaaatgagtcaaacactgagcctctaagtattaatatgcatgagagcttttaaaaagtcactgtctgtttattatggcttgctttggcactttctaaataatgagtgacgtaaagggtcttgttgcttcatagcatgccttaacttttttttggtcaccatcagagcaaatacacatcgaaatgttttatgttcctactttatcttataggttgatgatgagatgatgtctgcagcctcccagaagtttcccttcaatactcccaaaactaaggaaggctacttctaccgtcagatctttgaacgccattacccaggccgggctgattggctgactcattattggatgcctaagtggatcaatgctactgacccttctgcccgcactctgacccattataagtcagctgccaaagcttaggcactctctacactcttgtgtaaaagtaaatgtttcttccggctctgaaggtcgagacagcgacacaatcagaaagaatgagactcagccatcagtcacccaggcttacttaggcatgaaaagaaataaaagtttcacatctgaaatgcc

SEQ ID NO: 27 - DNA, 20 bp, synthetic construct
  misc_feature 1..20: note = synthetic
  source 1..20: mol_type = other DNA
ggttttctcgatgcctcctt

SEQ ID NO: 28 - DNA, 21 bp, synthetic construct
  misc_feature 1..21: note = synthetic
  source 1..21: mol_type = other DNA
tgtggctctgttacaatggtg

SEQ ID NO: 29 - DNA, 28 bp, synthetic construct
  misc_feature 1..28: note = synthetic
  source 1..28: mol_type = other DNA
gagttctaattctttttactgctcaatc

SEQ ID NO: 30 - DNA, 20 bp, synthetic construct
  misc_feature 1..20: note = synthetic
  source 1..20: mol_type = other DNA
agagcagagcagcgtagagc

SEQ ID NO: 31 - DNA, 20 bp, synthetic construct
  misc_feature 1..20: note = synthetic
  source 1..20: mol_type = other DNA
cacaggactagaacacctgc

SEQ ID NO: 32 - DNA, 19 bp, synthetic construct
  misc_feature 1..19: note = synthetic
  source 1..19: mol_type = other DNA
gctggtgaaaaggacctct

SEQ ID NO: 33 - DNA, 16 bp, synthetic construct
  misc_feature 1..16: note = synthetic
  source 1..16: mol_type = other DNA
ccaaaatatgtgtcca

SEQ ID NO: 34 - DNA, 16 bp, synthetic construct
  misc_feature 1..16: note = synthetic
  source 1..16: mol_type = other DNA
tattttatcacactcc

SEQ ID NO: 35 - DNA, 16 bp, synthetic construct
  misc_feature 1..16: note = synthetic
  source 1..16: mol_type = other DNA
gcttttaaatggtctt

SEQ ID NO: 36 - DNA, 16 bp, synthetic construct
  misc_feature 1..16: note = synthetic
  source 1..16: mol_type = other DNA
acgataacggtcagta